(19) United States
(12) Patent Application Publication
(10) Pub. No.: US 2024/0041889 A1
(43) Pub. Date: Feb. 8, 2024

(54) COMBINATION THERAPY WITH ADENOSINE RECEPTOR ANTAGONISTS

(71) Applicant: Teon Therapeutics, Inc., Redwood City, CA (US)

(72) Inventors: PEIDONG FAN, Redwood City, CA (US); LINA YAO, Redwood City, CA (US); JIWEN LIU, Redwood City, CA (US); ELFATIH ELZEIN, Redwood City, CA (US)

(21) Appl. No.: 18/020,064

(22) PCT Filed: Aug. 6, 2021
(86) PCT No.: PCT/US2021/044935

§ 371 Date: Feb. 6, 2023

Related U.S. Application Data
(60) Provisional application No. 63/062,857, filed on Aug. 7, 2020.

(51) Int. Cl. A61K 31/522; A61P 35/00; A61K 39/395

(52) U.S. Cl. CPC A61K 31/522; A61P 35/00; A61K 39/39558

(57) ABSTRACT

Disclosed herein are compounds, compositions, formulations, and methods for modulating the A_{2B} adenosine receptor in combination with immune checkpoint inhibitors in mammals with cancer.

### CROSS REFERENCE TO RELATED APPLICATIONS

This application claims the benefit of priority under 35 U.S.C. § 119(e) to U.S. Provisional Application No. 63/062,857, filed Aug. 7, 2020, which is incorporated herein by reference in its entirety.

### FIELD

Described herein are A_{2B} adenosine receptor antagonists, methods of making such compounds, pharmaceutical compositions and medicaments comprising such compounds, and methods of using such compounds alone or in combination with immune checkpoint inhibitors in the treatment of cancer in mammals.

### BACKGROUND

Adenosine, an endogenous nucleoside, ubiquitously exists inside and outside of living cells. It plays multiple physiological roles to maintain the homeostasis of cells, tissues, and organs. Adenosine can exert its biological effects by interacting with a family of adenosine receptors known as A₁, A_{2A}, A_{2B}, and A₃ adenosine receptors. A₁ adenosine receptors mediate mechanisms of tissue protection, especially for cardioprotection. A_{2A} adenosine receptors modulate coronary vasodilation and cancer immunity. A_{2B} adenosine receptors play a role in signaling pathways.

Some A_{2B} adenosine receptor antagonists are relatively insoluble in aqueous media and/or difficult to formulate using conventional pharmaceutical excipients, and thus can be difficult to formulate in a manner that provides reproducible plasma levels of the compound in mammals, in particular humans. A need exists for improving the bioavailability A_{2B} adenosine receptor antagonists.

### SUMMARY

In one aspect, described herein is a method for treating cancer in a mammal, the method comprising administering to the mammal a A_{2B} adenosine receptor antagonists and at least one immune checkpoint inhibitor.

In another aspect, described herein is a method for treating cancer in a mammal, the method comprising administering to the mammal Compound 1, or a pharmaceutically acceptable salt or solvate thereof, or a prodrug of Compound 1, or a pharmaceutically acceptable salt or solvate thereof, and at least one immune checkpoint inhibitor, wherein Compound 1 has the following structure:

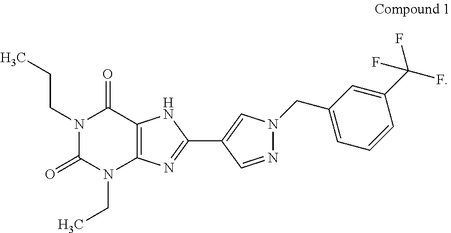 [SMILES: CCCn1c(=O)c2[nH]c(-c3cnn(Cc4cccc(C(F)(F)F)c4)c3)nc2n(CC)c1=O]

In some embodiments, the cancer is a solid tumor.

In some embodiments, the cancer is bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, gastric cancer, rectal cancer, urothelial cancer, testis cancer, cervical cancer, vaginal cancer, vulvar cancer, head and neck cancer, or skin cancer. In some embodiments, the cancer is prostate cancer, breast cancer, colon cancer, or lung cancer. In some embodiments, the cancer is castration resistant prostate cancer. In some embodiments, the cancer is breast cancer. In some embodiments, the cancer is a sarcoma, carcinoma, or lymphoma.

In some embodiments, the immune checkpoint inhibitor is an anti-PD-1 agent or an anti-PD-L1 agent. In some embodiments, the anti-PD-1 agent or anti-PD-L1 agent is nivolumab, pembrolizumab, cemiplimab, labrolizumab, avelumab, durvalumab or atezolizumab.

In some embodiments, the mammal is a human.

In one aspect, described herein is a method of modulating the A_{2B} adenosine receptor in a mammal comprising administering to the mammal a compound described herein, or any pharmaceutically acceptable salt or solvate thereof.

In any of the aforementioned aspects are further embodiments in which an effective amount of each therapeutic agent in the combination therapies described herein, is: (a) systemically administered to the mammal; and/or (b) administered orally to the mammal; and/or (c) intravenously administered to the mammal; and/or (d) administered by injection to the mammal.

In any of the aforementioned aspects are further embodiments comprising single administrations of effective amounts of each therapeutic agent, including further embodiments in which each therapeutic agent is administered once a day to the mammal or each therapeutic agent is administered to the mammal multiple times over the span of one day. In some embodiments, each therapeutic agent is administered on a continuous dosing schedule. In some embodiments, each therapeutic agent is administered on a continuous daily dosing schedule.

In some embodiments, provided is a method for treating cancer in a mammal, the method comprising administering to the mammal Compound 1, or a pharmaceutically acceptable salt or solvate thereof, or a prodrug of Compound 1 (e.g., prodrug of Formula (I), (II), (IIa), (III), (A), and/or (B) as described herein), or a pharmaceutically acceptable salt or solvate thereof. In some embodiments, the cancer is a solid tumor. In some embodiments, the cancer is bladder cancer, colon cancer, brain cancer, breast cancer, endometrial cancer, heart cancer, kidney cancer, lung cancer (e.g., non-small cell lung cancer, small cell lung cancaer), liver cancer, uterine cancer, blood and lymphatic cancer, ovarian cancer, pancreatic cancer, prostate cancer, thyroid cancer, gastric cancer (e.g., stomach cancer), rectal cancer, urothelial cancer, testes/testicular cancer, cervical cancer, vaginal cancer, vulvar cancer, head and neck cancer, or skin cancer. In some embodiments, the cancer is prostate cancer, breast cancer, colon cancer, or lung cancer. In some embodiments, the cancer is castration resistant prostate cancer. In some embodiments, the cancer is breast cancer. In some embodiments, the cancer is a sarcoma, carcinoma, or lymphoma.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE DRAWINGS

FIG. 1: Illustrates the effects of Compound 1, the anti-PD-1 antibody RMP1-14, and the combination of the two agents in the CT26 (Hot) Model.

FIG. 2: Illustrates the effects of Compound 1, the anti-PD-1 antibody RMP1-14, and the combination of the two agents in the B16F10 Melanoma (Cold) Model.

FIG. 3: Panels a., b., c., d., and e. in FIG. 3 illustrates the effects of Compound 1, the anti-PD-1 antibody RMP1-14, and the combination of the two agents on tumor-infiltrating immune cells in a MC38 model.

### DETAILED DESCRIPTION

Disclosed herein are compounds, compositions, formulations, and methods related to A_{2B} adenosine receptor antagonists. For example, the compounds, compositions, and/or formulations disclosed herein can be used in a method of treating a condition in a subject in need thereof. The condition can be cardiovascular diseases, chronic and acute liver disease, lung disease, renal disease, diabetes, obesity, and/or cancer.

8-(1-(3-(Trifluoromethyl)benzyl)-1H-pyrazol-4-yl)-3-ethyl-1-propyl-1H-purine-2,6(3H,7H)-dione (Compound 1) is an A_{2B} adenosine receptor antagonist, which is a xanthine unsubstituted at 7-position. It can be relatively insoluble in aqueous media and difficult to formulate using conventional pharmaceutical excipients, and thus can be difficult to formulate in a manner that provides reproducible plasma levels of the compound undergoing evaluation in mammals, in particular humans. Accordingly, new prodrugs of the A_{2B} adenosine receptor antagonist can be developed to improve the formulation, pharmacokinetic profile, and/or bioavailability the A_{2B} adenosine receptor antagonist.

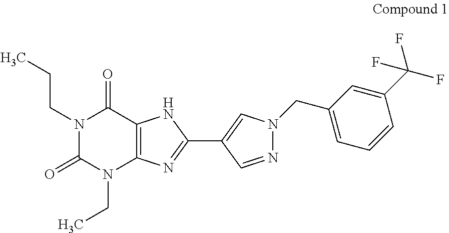 [SMILES: CCCn1c(=O)c2[nH]c(-c3cnn(Cc4cccc(C(F)(F)F)c4)c3)nc2n(CC)c1=O]

In some cases, prodrugs can be hydrolyzed by esterase (e.g., in gastrointestinal tract and/or in blood) and converted into Compound 1 in an aqueous solution. In some cases, acid labile prodrugs can be converted into Compound 1 in an acidic environment (e.g., in the stomach). In some cases, prodrugs, which are stable in the acidic environment and/or stable against hydrolysis by esterase, may not be a good prodrug candidate for Compound 1.

In one aspect, the compounds, compositions, and/or formulations disclosed herein can be used to treat cancer. On endothelial cells, for example, adenosine can bind to the A_{2B} adenosine receptors, thereby stimulating angiogenesis. On T cells, A_{2B} adenosine receptor stimulation can lead to type I protein kinase A (PKA) isoform activation that can hamper T cell activation through inhibition of T-cell antigen receptor (TCR) proximal kinases Lck and Fyn.

The pro-metastatic Fra-1 transcription factor can also induce A_{2B} adenosine receptor expression on cancer cells, and thus A_{2B} adenosine receptor antagonist can inhibit metastasis of Fra-1-expressing cells. A_{2B} adenosine receptor signaling activation can impair antigen presentation and can also inhibit signal transducer and activator of transcription 1 (STAT1) activation. A_{2B} has an effect on immunity which is mediated by dendritic cells (DCs), Myeloid-derived-suppressor cells (MDSCs) and regulatory T-cells (Tregs). A_{2B}/cAMP/PKA potently dampens the immune response via inhibiting DCs function and stimulating immunosuppressive cells, such as myeloid-derived suppressor cells (MDSCs) and Tregs. A_{2B} activation impairs tumor antigen presentation on myeloid cells and DCs, decreases production of pro-inflammatory cytokines (TNF-α and IL-12) and increases immunosuppressive IL-10, resulting in a lower expression of CD86 and MHC class II and less efficient CD4+ T cell stimulation and antitumor responses. A_{2B} activation promotes the expansion of MDSCs, which potently suppresses antitumor T cell response and promotes angiogenesis. A_{2B} activation also stimulates Tregs differentiation to suppress T cell function. Besides, A_{2B} activation can contribute to the pro-angiogenic effects by increasing the production of vascular endothelial growth factor (VEGF) in endothelial cells. Taken together, A_{2B} plays an important role in tumor cell proliferation, angiogenesis, metastasis and immune suppression. The diversity of signaling and biological activities of A_{2B} adenosine receptor can render it an attractive cancer target to promote anti-tumor immunity and suppress tumor cell metastasis.

In another aspect, the compounds, compositions, and/or formulations disclosed herein can be used to treat fibrosis. A commonly ingested adenosine receptor antagonist, caffeine, can block the development of hepatic fibrosis, an effect that may explain the epidemiologic finding that coffee drinking, in a dose-dependent fashion, can reduce the likelihood of death from liver disease. A_{2B} adenosine receptors can also play a role in the pathogenesis of interstitial fibrosis. Adenosine, acting at A_{2B} adenosine receptors, can stimulate hepatic stellate cell-mediated fibrosis of the liver by increasing production of collagen I and III via two distinct mitogen-activated protein kinase (MAPK)-dependent pathways, extracellular signal-regulated kinase ½ (ERK1/2) and p38MAPK, respectively. Over-activation of A_{2B} adenosine receptors can be involved in liver, lung and heart fibrosis. Accordingly, A_{2B} adenosine receptors may be a good therapeutic target for fibrosis of the liver, lungs, heart, kidney, and/or skin. Applicant has found that Compound 1 reduces fibrosis in a MC38 tumor model, suggesting that Compound 1 could improve the tumor microenvironment for T cell function and infiltration and therapeutic antibody (such as anti-PD-1 antibody) penetration.

In another aspect, the compounds, compositions, and/or formulations disclosed herein can be used to treat diabetes and/or obesity. Insensitivity to insulin can exacerbate diabetes and/or obesity. Insulin sensitivity can be decreased by the interaction of adenosine with A_{2B} adenosine receptors. Thus, blocking the A_{2B} adenosine receptors of individuals with diabetes and/or obesity can benefit patients with these disorders.

In another aspect, the compounds, compositions, and/or formulations disclosed herein can be used to treat neurological disorders, such as dementias and Alzheimer's disease. Adenosine acting at A_{2B} adenosine receptors can over-stimulate cerebral interleukin 6 (IL-6), a cytokine associated with dementias and Alzheimer's disease. Inhibiting the binding of adenosine to A_{2B} adenosine receptors can therefore mitigate those neurological disorders that are produced by IL-6.

In another aspect, the compounds, compositions, and/or formulations disclosed herein can be used to treat type I hypersensitivity disorders, such as chronic obstructive pulmonary disease (COPD), asthma, hay fever, and atopic eczema. These type I hypersensitivity disorders can be stimulated by mast cells binding to A_{2B} adenosine receptors. Therefore, blocking A_{2B} adenosine receptors can provide a therapeutic benefit against such disorders.

In another aspect, the compounds, compositions, and/or formulations disclosed herein can be used to treat irritable bowel disease (IBD) and/or colitis. Certain hypersensitivity disorders can be stimulated by mast cells binding to A_{2B} adenosine receptors. Therefore, blocking A_{2B} adenosine receptors can provide a therapeutic benefit against IBD and/or colitis.

### Definitions

Unless defined otherwise, all technical and scientific terms used herein have the same meaning as commonly understood by one of the ordinary skill in the art to which this disclosure belongs. Although any methods and materials similar or equivalent to those described herein can be used in the practice or testing of the formulations or unit doses herein, some methods and materials are now described. Unless mentioned otherwise, the techniques employed or contemplated herein are standard methodologies. The materials, methods and examples are illustrative only and not limiting.

The section headings used herein are for organizational purposes only and are not to be construed as limiting the subject matter described.

The details of one or more inventive embodiments are set forth in the accompanying drawings, the claims, and the description herein. Other features, objects, and advantages of the inventive embodiments disclosed and contemplated herein can be combined with any other embodiment unless explicitly excluded.

The open terms for example “contain,” “containing,” “include,” “including,” and the like mean comprising, and are not limiting.

The singular forms “a,” “an,” and “the” are used herein to include plural references unless the context clearly dictates otherwise.

Unless otherwise indicated, some embodiments herein contemplate numerical ranges. When a numerical range is provided, unless otherwise indicated, the range can include the range endpoints. Unless otherwise indicated, numerical ranges can include all values and subranges therein as if explicitly written out.

The term “about” in relation to a reference numerical value can include a range of values plus or minus 10% from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

The term “prodrug” refers to any compound that becomes an active form of a drug (e.g., Compound I) when administered to a subject, e.g., upon metabolic processing of the prodrug.

Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of compound 1 described herein include, but are not limited to, compounds where the nitrogen atom is incorporated into an alkyl carbamate, (acyloxy)alkyl carbamate, acyloxyalkyl ester, alkoxycarbonyloxyalkyl ester, alkyl ester, aryl ester, phosphate ester, sugar ester, ether, N-acyloxyalkoxycarbonyl, N-acyloxyakyl, dihydropyridinepyridinium salt system (redox systems), (phosphoryloxy)methyl carbamate, (acyloxy)alkyl carbamate, and the like.

In some embodiments, prodrugs of Compound 1 are formed by N-acyloxyalkylation, N-hydroxyalkylation, N-(phosphoryloxy)alkylation, N-acyloxyalkylation, N-hydroxyalkylation, N-(phosphoryloxy)alkylation, N-acylation (amides and carbamates), N-(oxodioxolenyl)methylation, and the like.

The term “pharmaceutically acceptable” component is one that is suitable for use with humans and/or animals without undue adverse side effects (such as toxicity, irritation, and allergic response) commensurate with a reasonable benefit/risk ratio.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation and/or amelioration of the signs, symptoms, or causes of a disease, slowing of disease progression, or any other desired alteration of a biological system. For example, an “effective amount” for therapeutic uses is the amount of compound as disclosed herein required to provide a clinically significant decrease in disease symptoms. An appropriate “effective” amount in any individual case is optionally determined using techniques, such as a dose escalation study.

The term “treating” or “treatment” encompasses administration of at least one compound disclosed herein, or a pharmaceutically acceptable salt thereof, to a mammalian subject, particularly a human subject, in need of such an administration and includes (i) arresting the development of clinical symptoms of the disease, such as cancer, (ii) bringing about a regression in the clinical symptoms of the disease, such as cancer, and/or (iii) prophylactic treatment for preventing the onset of additional symptoms of the disease, such as cancer.

The term “subject” refers to a mammal that has been or will be the object of treatment, observation or experiment.

The term “mammal” is intended to have its standard meaning, and encompasses for example humans, dogs, cats, sheep, and cows. The methods described herein can be useful in both human therapy and veterinary applications. In some embodiments, the mammal is a human.

The term “derivative” can be used interchangeably with the term “analog.” Compound 1 can be a derivative or analog if 1, 2, 3, 4, or 5 atoms of compound 1 is replaced by another atom or a functional group (e.g., amino, halo, substituted or unsubstituted alkyl, substituted or unsubstituted aryl, substituted or unsubstituted arylalkyl, or substituted or unsubstituted cycloalkyl) to form the compounds of the disclosure.

The term “solvate” can include, but is not limited to, a solvate that retains one or more of the activities and/or properties of the compound and that is not undesirable. Examples of solvates include, but are not limited to, a compound in combination with water, isopropanol, ethanol, methanol, DMSO, ethyl acetate, acetic acid, ethanolamine, or combinations thereof.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

The term “salt” can include, but are not limited to, salts that retain one or more of the activities and properties of the free acids and bases and that are not undesirable. Illustrative examples of salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methanesulfonates, propanesulfonates, naphthalene-1-sulfonates, naphthalene-2-sulfonates, and mandelates.

It should be understood that a reference to a pharmaceutically acceptable salt includes the solvent addition forms. In some embodiments, solvates contain either stoichiometric or non-stoichiometric amounts of a solvent, and are formed during the process of isolating or purifying the compound with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

Unless otherwise indicated, whenever there is a stereocenter in a structure disclosed or illustrated herein, the stereocenter can be R or S in each case.

Individual stereoisomers are obtained, if desired, by methods such as, stereoselective synthesis and/or the separation of stereoisomers by chiral chromatographic columns. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In another embodiment, the compounds described herein are labeled isotopically (e.g. with a radioisotope) or by another other means.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, one or more hydrogen atoms of the compounds described herein is replaced with deuterium.

The term “amino” refers to functional groups that contain a basic nitrogen atom with a lone pair. For example, amino can include the radical

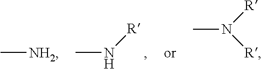

wherein each R′ is independently H, halo, alkyl, aryl, arylalkyl, cycloalkyl, or acyl.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, 4 carbon atoms, 5 carbon atoms,6 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Alternatively, an alkyl includes, but is not limited to, methyl, ethyl, propan-1-yl, propan-2-yl, butan-1-yl, butan-2-yl, 2-methyl-propan-1-yl, 2-methyl-propan-2-yl, and the like. Typical alkyl groups include, but are in no way limited to, methyl, ethyl, propyl, isopropyl, butyl, isobutyl, sec-butyl, tertiary butyl, pentyl, neopentyl, or hexyl.

The term “lower alkyl” can refer to a monoradical branched or unbranched saturated hydrocarbon chains having 1, 2, 3, 4, 5, or 6 carbon atoms, such as methyl, ethyl, n-propyl, iso-propyl, n-butyl, iso-butyl, t-butyl, n-hexyl, and the like.

“Alkylsulfone” refers to the group R—S(O)₂— where R is alkyl as defined herein.

“Alkylsulfoxide” refers to the group R—S(O)— where R is alkyl as defined herein.

“Alkylthio” refers to the group R—S— where R is alkyl as defined herein.

In some embodiments, when an alkyl is unsaturated, then the alkyl is an alkenyl or alkynyl.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂. Alternatively, an alkenyl includes, but is not limited to, ethenyl, prop-1-en-1-yl, prop-1-en-2-yl, prop-2-en-1-yl (allyl), but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, and the like.

The term “alkynyl” refers to a type of alkyl group in which at least one carbon-carbon triple bond is present. In one embodiment, an alkynyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH. Alternatively, an alkynyl includes, but is not limited to, ethynyl, prop-1-yn-1-yl, prop-2-yn-1-yl, but-1-yn-1-yl, but-1-yn-3-yl, but-3-yn-1-yl; and the like.

An “alkoxy” group refers to a (alkyl)O— group, where alkyl is as defined herein.

The term “fluoroalkyl” refers to an alkyl in which one or more hydrogen atoms are replaced by a fluorine atom. In one aspect, a fluoroalkyl is a C₁-C₆fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

An “fluoroalkoxy” group refers to a (fluoroalkyl)O— group, where fluoroalkyl is as defined herein.

The term “halo” or “halogen” refers to fluorine, chlorine, bromine or iodine.

The term “heteroalkyl” refers to an alkyl group in which one or more skeletal atoms of the alkyl are selected from an atom other than carbon, e.g., oxygen, nitrogen (e.g. —NH—, —N(alkyl)-), sulfur (—S—, —S(O)—, —S(O)₂—), phosphorus (—PH—, —P(O)₂—), or combinations thereof (e.g. —O—P(O)₂—). A heteroalkyl is attached to the rest of the molecule at a carbon atom of the heteroalkyl. In one aspect, a heteroalkyl is a C₁-C₆heteroalkyl. In some embodiments,

As used herein, the term “aryl” refers to an aromatic ring wherein each of the atoms forming the ring is a carbon atom. Typical aryl groups include, but are not limited to, phenyl, naphthyl, fluorenyl, indanyl, indenyl, and the like. In one some embodiments, aryl is phenyl or a naphthyl. In some embodiments, an aryl is a phenyl. In some embodiments, an aryl is a C₆-C₁₀aryl.

“Aryloxy” refers to the group Ar—O— where Ar is aryl as defined herein.

“Arylsulfone” refers to the group Ar—S(O)₂— where Ar is aryl as defined herein.

“Arylsulfoxide” refers to the group R—S(O)— where Ar is aryl as defined herein.

“Arylthio” refers to the group Ar—S— where Ar is aryl as defined herein.

The terms “heteroaryl” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

The term “arylalkyl” refers to an alkyl that is substituted with an aryl group. Typical arylalkyl groups include, but are not limited to, benzyl, 2-phenylethan-1-yl, naphthylmethyl, 2-naphthylethan-1-yl, and the like.

The term “heteroarylalkyl” refers to an alkyl that is substituted with a heteroaryl group.

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are monocyclic, bicyclic (spirocyclic, fused or bridged), or polycyclic. Cycloalkyl groups include groups having from 3 to 10 ring atoms (i.e. (C₃-C₁₀) cycloalkyl). In some embodiments, a cycloalkyl is a (C₃-C₆) cycloalkyl. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicyclo[1.1.1]pentyl. In some embodiments, a cycloalkyl is a C₃-C₆cycloalkyl. In some embodiments, a cycloalkyl is a monocyclic cycloalkyl. Monocyclic cycloalkyls include, but are not limited to, cyclopropyl, cyclobutyl, cyclopentyl, cyclohexyl, cycloheptyl, and cyclooctyl. Polycyclic cycloalkyls include, for example, adamantyl, norbornyl (i.e., bicyclo[2.2.1]heptanyl), norbornenyl, decalinyl, 7,7-dimethyl-bicyclo[2.2.1]heptanyl, and the like.

In some embodiments, a cycloalkyl is partially unsaturated (“cycloalkenyl”, including but not limited to, cyclobut-1-en-1-yl, cyclobut-1-en-3-yl, cyclobuta-1,3-dien-1-yl, and the like).

A “heterocycloalkyl” or “heteroalicyclic” or “heterocyclyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “acyl” can refer to —C(O)R′, in which R′ is hydrogen, alkyl, substituted alkyl, arylalkyl, substituted arylalkyl, cycloalkyl, substituted cycloalkyl, aryl, or substituted aryl.

The term “substituted” can refer to a group in which one or more hydrogen atoms are each independently replaced with the same or different substituent(s). Typical substituents include, but are

The term “substituted” or “optionally substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some embodiments, optional substituents are independently selected from halo, alkyl, heteroalkyl, aryl, heteroaryl, arylalkyl, heteroarylalkyl, cycloalkyl, heterocycloalkyl, or acyl. In some other embodiments, optional substituents are independently selected from D, halogen, —CN, —NH₂,

—NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

### Prodrugs

In one aspect, described herein is a compound (e.g., a prodrug of Compound 1) represented by Formula (A):

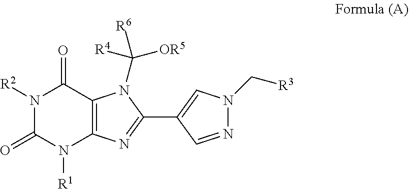

or a pharmaceutically acceptable salt or solvate thereof, wherein:
R¹ and R² are each independently selected from hydrogen, and substituted or unsubstituted alkyl;
R³ is selected from substituted or unsubstituted phenyl, and substituted or unsubstituted heteroaryl, wherein if R³ is substituted then R³ is substituted with one or more groups selected from halogen, —CN, —OH, C₁-C₄alkyl, C₂-C₄alkenyl, C₂-C₄alkynyl, C₁-C₄alkoxy, C₁-C₄fluoroalkyl, C₁-C₄fluoroalkoxy, and substituted or unsubstituted C₁-C₄heteroalkyl;
R⁴ is substituted or unsubstituted alkyl;
R⁶ is hydrogen or substituted or unsubstituted alkyl;
or R⁴ and R⁶ are taken together with the carbon atom to which they are attached to form a carbonyl (C═O);
or R⁴ and R⁶ are taken together with the carbon atom to which they are attached to form a ring that is a substituted or unsubstituted C₃-C₁₀cycloalkyl, or substituted or unsubstituted C₂-C₁₀heterocycloalkyl, wherein if the ring is substituted then it is substituted with one or more R¹⁵;
   R¹⁵ is hydrogen, substituted or unsubstituted alkyl, substituted or unsubstituted heteroalkyl, substituted or unsubstituted phenyl, substituted or unsubstituted heteroaryl, -alkyl-(substituted or unsubstituted phenyl), -alkyl-(substituted or unsubstituted heteroaryl), —C(═O)R¹⁶, —C(═O)—OR¹⁶, —C(═O)N(R¹⁶)₂;
   each R¹⁶ is independently selected from hydrogen and substituted or unsubstituted alkyl;
R⁵ is hydrogen, R⁷, —C(═O)R⁷, —C(═O)—OR⁷, —C(═O)N(R⁷)(R⁸), —C(═O)—SR⁷, or —P(═O)(OR⁹)₂;
or R⁴ and R⁵ are taken together with the atoms to which they are attached to form a substituted or unsubstituted C₂-C₁₀heterocycloalkyl;
R⁷ is substituted or unsubstituted alkyl, substituted or unsubstituted heteroalkyl, substituted or unsubstituted C₃-C₁₀cycloalkyl, substituted or unsubstituted C₂-C₁₀heterocycloalkyl, substituted or unsubstituted phenyl, substituted or unsubstituted heteroaryl, -alkyl-(substituted or unsubstituted phenyl), -alkyl-(substituted or unsubstituted heteroaryl),
   alkyl-(substituted or unsubstituted cycloalkyl),-alkyl-(substituted or unsubstituted heterocycloalkyl), —(C(R¹⁰)₂O)ₘ—R¹¹, —(CH₂CH₂O)ₙ—R¹¹, or —(C(R¹⁰)₂)ₚ—OR¹¹;
R⁸ is hydrogen or alkyl;
or R⁷ and R⁸ are taken together with the nitrogen atom to which they are attached to form a substituted or unsubstituted C₂-C₁₀heterocycloalkyl;
each R⁹ is independently selected from hydrogen and alkyl;
each R¹⁰ is independently selected from hydrogen and alkyl;
R¹¹ is hydrogen, substituted or unsubstituted alkyl, substituted or unsubstituted heteroalkyl, substituted or unsubstituted C₂-C₁₀heterocycloalkyl, —C(═O)R¹², —C(═O)—OR¹², —C(═O)N(R¹²)(RI), —C(═O)—SR¹², or —P(═O)(OR⁹)₂;
R¹² is hydrogen, substituted or unsubstituted alkyl, substituted or unsubstituted heteroalkyl, substituted or unsubstituted C₃-C₁₀cycloalkyl, substituted or unsubstituted
   C₂-C₁₀heterocycloalkyl, substituted or unsubstituted phenyl, substituted or unsubstituted heteroaryl, -alkyl-(substituted or unsubstituted phenyl), or -alkyl-(substituted or unsubstituted heteroaryl);
m is 1, 2, 3, 4, 5, or 6;
n is 1, 2, 3, 4, 5, or 6;
p is 1, 2, 3, 4, 5, or 6;
wherein substituted means that the referenced group is substituted with one or more additional groups individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone.

In some embodiments, m is 1, 2, 3, 4, 5, or 6. In some embodiments, m is 1, 2, 3, 4, or 5. In some embodiments, m is 1, 2, 3, or 4. In some embodiments, m is 1, 2, or 3. In some embodiments, m is 1 or 2. In some embodiments, m is 1. In some embodiments, m is 2, 3, 4, 5, or 6.

In some embodiments, n is 1, 2, 3, 4, 5, or 6. In some embodiments, n is 1, 2, 3, 4, or 5. In some embodiments, n is 1, 2, 3, or 4. In some embodiments, n is 1, 2, or 3. In some embodiments, n is 1 or 2. In some embodiments, n is 2 or 3. In some embodiments, n is 1. In some embodiments, n is 2. In some embodiments, n is 3. In some embodiments, n is 2, 3, 4, 5, or 6.

In some embodiments, p is 1, 2, 3, 4, 5, or 6. In some embodiments, p is 1, 2, 3, 4, or 5. In some embodiments, p is 1, 2, 3, or 4. In some embodiments, p is 1, 2, or 3. In some embodiments, p is 1 or 2. In some embodiments, p is 1. In some embodiments, p is 2, 3, 4, 5, or 6.

In some embodiments, R¹ and R² are each independently selected from substituted or unsubstituted C₁-C₆alkyl. In some embodiments, R¹ and R² are each independently selected from unsubstituted C₁-C₃alkyl. In some embodiments, R¹ is ethyl. In some embodiments, R² is n-propyl. In some embodiments, R¹ is ethyl and R² is n-propyl.

In some embodiments, R³ is selected from substituted or unsubstituted phenyl. In some embodiments, R³ is substituted phenyl. In some embodiments, R³ is phenyl substituted by one or more groups independently selected from halogen, C₁-C₄ alkyl, or C₁-C₄ fluoroalkyl. In some embodiments, R³ is phenyl substituted by one or more groups independently selected from C₁-C₄ fluoroalkyl. In some embodiments, R³ is selected from phenyl substituted with one, two, or three —CF₃ substituents. In some embodiments, R³ is selected from phenyl substituted with one —CF₃ substituent. In some embodiments, R³ is

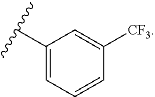 [SMILES: *c1cccc(C(F)(F)F)c1]

In some embodiments, R¹ and R² are each independently selected from substituted or unsubstituted C₁-C₆alkyl; R³ is selected from substituted or unsubstituted phenyl.

In some embodiments, R¹ and R² are each independently selected from methyl, ethyl, n-propyl, iso-propyl, n-butyl, iso-butyl, tert-butyl, n-pentyl, tert-pentyl, neopentyl, isopentyl, sec-pentyl, 3-pentyl, n-hexyl, isohexyl, 3-methylpentyl, 2,3-dimethylbutyl, and neohexyl.

In some embodiments, R¹ is ethyl; R² is n-propyl; and R³ is 3-(trifluoromethyl)phenyl.

In some embodiments, R⁴ is C₁-C₆alkyl and R⁶ is selected from hydrogen, and C₁-C₆alkyl. In some embodiments, R⁴ and R⁶ are taken together with the carbon atom to which they are attached to form a carbonyl (C═O).

In some embodiments, R⁴ is methyl, ethyl, or n-propyl and R⁶ is selected from hydrogen, methyl, ethyl, and n-propyl. In some embodiments, R⁴ is methyl or ethyl. In some embodiments, R⁶ is hydrogen. In some embodiments, R⁴ is methyl or ethyl; and R⁶ is hydrogen.

In some embodiments, R⁵ is R⁷. In some embodiments, R⁵ is —(C═O)R⁷. In some embodiments, R⁵ is —(C═O)—OR⁷.

In some embodiments, R⁵ is R⁷; R⁷ is C₁-C₆alkyl, substituted or unsubstituted C₁-C₆heteroalkyl, substituted or unsubstituted monocyclic C₃-C₈cycloalkyl, substituted or unsubstituted bicyclic C₅-C₁₀cycloalkyl, substituted or unsubstituted monocyclic C₂-C₈heterocycloalkyl, substituted or unsubstituted bicyclic C₅-C₁₀heterocycloalkyl, substituted or unsubstituted phenyl, substituted or unsubstituted monocyclic heteroaryl, —CH₂-(substituted or unsubstituted phenyl), —CH₂-(substituted or unsubstituted heteroaryl), —CH₂-(substituted or unsubstituted C₂-C₈heterocycloalkyl), —CH(R¹⁰)O—R¹¹, —(CH₂CH₂O)ₙ—R¹¹, or —(C(R¹⁰)₂)ₚ—OR¹¹; each R¹⁰ is independently selected from hydrogen and methyl; R¹¹ is hydrogen, C₁-C₆alkyl, substituted or unsubstituted C₁-C₆heteroalkyl, substituted or unsubstituted C₂-C₁₀heterocycloalkyl, —C(═O)R¹², —C(═O)—OR¹², —C(═O)N(R¹²)(R′), —C(═O)—SR¹², or —P(═O)(OR⁹)₂.

In some embodiments, R⁷ is C₁-C₆alkyl, substituted or unsubstituted C₁-C₆heteroalkyl, —CH₂-(substituted or unsubstituted phenyl), —CH₂-(substituted or unsubstituted heteroaryl), —CH₂-(substituted or unsubstituted C₂-C₈heterocycloalkyl), —CH(R¹⁰)O—R¹¹, or —(CH₂CH₂O)ₙ—R¹¹; R¹⁰ is hydrogen and methyl; R¹¹ is hydrogen, C₁-C₆alkyl, substituted or unsubstituted C₁-C₆heteroalkyl, substituted or unsubstituted C₂-C₁₀heterocycloalkyl, —C(═O)R¹², —C(═O)—OR¹², —C(═O)N(R¹²)(RI), —C(═O)—SR¹², or —P(═O)(OH)₂.

In some embodiments, R⁷ is C₁-C₆alkyl. In some embodiments, R⁷ is methyl, ethyl, n-propyl, isopropyl, n-butyl, or n-pentyl.

In some embodiments, R⁷ is —CH(R¹⁰)O—R¹¹, wherein R¹¹ is —C(═O)R¹², and wherein R¹² is unsubstituted alkyl, unsubstituted C₃-C₁₀cycloalkyl. In some embodiments, R¹² is methyl, ethyl, n-propyl, n-butyl, or n-pentyl. In some embodiments, R¹² is cyclopropyl, cyclobutyl, cyclopentyl, or cyclohexyl.

In some embodiments, R⁷ is —CH(R¹⁰)O—R¹¹, wherein R¹¹ is —P(═O)(OR⁹)₂. In some embodiments, R⁹ is hydrogen.

In some embodiments, R⁷ is —(CH₂CH₂O)ₙ—R¹¹, wherein R¹¹ is unsubstituted alkyl. In some embodiments, R¹¹ is methyl, ethyl, n-propyl, n-butyl, or n-pentyl.

In some embodiments, R⁷ is —CH₂-(substituted or unsubstituted C₂-C₈heterocycloalkyl). In some embodiments, R⁷ is —CH₂-(substituted C₅-C₆heterocycloalkyl).

In some embodiments, R⁷ is

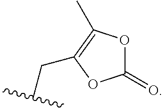 [SMILES: *Cc1oc(=O)oc1C]

In some embodiments, R⁷ is substituted or unsubstituted C₃-C₁₀ cycloalkyl. In some embodiments, R⁷ is unsubstituted C₃-C₁₀ cycloalkyl. In some embodiments, R⁷ is monocyclic C₃-C₁₀ cycloalkyl. In some embodiments, R⁷ is cyclopropyl, cyclobutyl, cyclopentyl, or cyclohexyl. In some embodiments, R⁷ is cyclohexyl. In some embodiments, R⁷ is spirocyclic C₃-C₁₀ cycloalkyl. In some embodiments, R⁷ is adamantyl.

In some embodiments, R⁴ is methyl or ethyl; R⁵ is hydrogen, R⁷, —C(═O)R⁷, —C(═O)—OR⁷, —C(═O)N(R⁷)(R⁸), —C(═O)—SR⁷, or —P(═O)(OR⁹)₂; R⁷ is C₁-C₆alkyl, substituted or unsubstituted C₁-C₆heteroalkyl, substituted or unsubstituted monocyclic C₃-C₈cycloalkyl, substituted or unsubstituted bicyclic C₅-C₁₀cycloalkyl, substituted or unsubstituted monocyclic C₂-C₈heterocycloalkyl, substituted or unsubstituted bicyclic C₅-C₁₀heterocycloalkyl, substituted or unsubstituted phenyl, substituted or unsubstituted monocyclic heteroaryl, —CH₂-(substituted or unsubstituted phenyl), —CH₂-(substituted or unsubstituted heteroaryl), —CH₂-(substituted or unsubstituted C₂-C₈heterocycloalkyl), —CH(R¹⁰)O—R¹¹, —(CH₂CH₂O)ₙ—R¹¹, or —(C(R¹⁰)₂)ₚ—OR¹¹; each R¹⁰ is independently selected from hydrogen and methyl; R¹¹ is hydrogen, C₁-C₆alkyl, substituted or unsubstituted C₁-C₆heteroalkyl, substituted or unsubstituted C₂-C₁₀heterocycloalkyl, —C(═O)R¹², —C(═O)—OR¹², —C(═O)N(R¹²)(R⁸), —C(═O)—SR¹², or —P(═O)(OR⁹)₂.

In some embodiments, R⁵ is R⁷, —C(═O)R⁷, —C(═O)—OR⁷, —C(═O)N(R⁷)(R⁸), —C(═O)—SR⁷, or —P(═O)(OH)₂; R⁷ is C₁-C₆alkyl, substituted or unsubstituted C₁-C₆heteroalkyl, substituted or unsubstituted cyclohexyl, substituted or unsubstituted cyclopentyl, substituted or unsubstituted bicyclo[1.1.1]pentanyl, substituted or unsubstituted bicyclo[2.2.1]heptanyl, substituted or unsubstituted bicyclo[2.2.2]octanyl, substituted or unsubstituted bicyclo[3.2.1]octanyl, substituted or unsubstituted bicyclo[3.3.0]octanyl, substituted or unsubstituted bicyclo[4.3.0]nonanyl, or substituted or unsubstituted decalinyl, substituted or unsubstituted oxetanyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, substituted or unsubstituted thiomorpholinyl, substituted or unsubstituted phenyl, substituted or unsubstituted monocyclic heteroaryl, —CH₂-(substituted or unsubstituted phenyl), —CH₂-(substituted or unsubstituted heteroaryl), —CH₂-(substituted or unsubstituted C₂-C₈heterocycloalkyl), —CH(R¹⁰)O—R¹¹, —(CH₂CH₂O)ₙ—R¹¹, or —(C(R¹⁰)₂)ₚ—OR¹¹; each R¹⁰ is independently selected from hydrogen and methyl; R¹¹ is hydrogen, C₁-C₆alkyl, substituted or unsubstituted C₁-C₆heteroalkyl, substituted or unsubstituted C₂-C₁₀heterocycloalkyl, —C(═O)R¹², —C(═O)—OR¹², —C(═O)N(R¹²)(RI), —C(═O)—SR¹², or —P(═O)(OR⁹)₂.

In some embodiments, R⁵ is R⁷, wherein R⁷ is C₁-C₆alkyl. In some embodiments, R⁷ is methyl, ethyl, n-propyl, isopropyl, n-butyl, or n-pentyl.

In some embodiments, R⁵ is —C(═O)R⁷, wherein R⁷ is C₁-C₆alkyl or unsubstituted C₃-C₁₀cycloalkyl. In some embodiments, R⁷ is methyl, ethyl, n-propyl, isopropyl, n-butyl, or n-pentyl. In some embodiments, R⁷ is cyclopropyl, cyclobutyl, cyclopentyl, cyclohexyl, bicyclo[1.1.1]pentanyl, s bicyclo[2.2.1]heptanyl, bicyclo[2.2.2]octanyl, bicyclo[3.2.1]octanyl, bicyclo[3.3.0]octanyl, or bicyclo[4.3.0]nonanyl.

In some embodiments, R⁵ is —C(═O)—OR⁷, wherein R⁷ is C₁-C₆alkyl or unsubstituted C₃-C₁₀cycloalkyl. In some embodiments, R⁷ is methyl, ethyl, n-propyl, isopropyl, n-butyl, or n-pentyl. In some embodiments, R⁷ is cyclopropyl, cyclobutyl, cyclopentyl, cyclohexyl, bicyclo[1.1.1]pentanyl, s bicyclo[2.2.1]heptanyl, bicyclo[2.2.2]octanyl, bicyclo[3.2.1]octanyl, bicyclo[3.3.0]octanyl, or bicyclo[4.3.0]nonanyl.

In some embodiments, the compound (e.g., a prodrug of Compound 1) has the following structure of Formula (III):

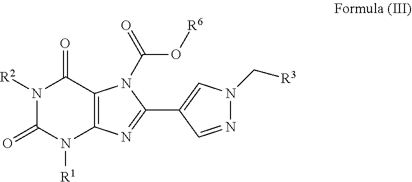

or a pharmaceutically acceptable salt or solvate thereof.

In some embodiments, R¹ and R² are each independently selected from substituted or unsubstituted C₁-C₆alkyl; R³ is selected from substituted or unsubstituted phenyl.

In some embodiments, R¹ and R² are each independently selected from methyl, ethyl, n-propyl, iso-propyl, n-butyl, iso-butyl, tert-butyl, n-pentyl, tert-pentyl, neopentyl, isopentyl, sec-pentyl, 3-pentyl, n-hexyl, isohexyl, 3-methylpentyl, 2,3-dimethylbutyl, and neohexyl.

In some embodiments, R¹ is ethyl; R² is n-propyl; and R³ is 3-(trifluoromethyl)phenyl.

In some embodiments, a prodrug of Compound 1 is a compound represented by Formula (III):

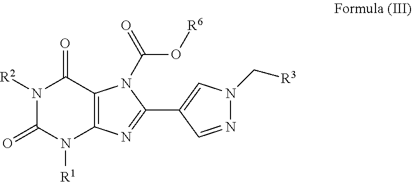

or a pharmaceutically acceptable salt thereof;
wherein
R¹ and R² are each independently selected from hydrogen and substituted or unsubstituted C₁-C₆alkyl;
R³ is selected from substituted and unsubstituted phenyl, wherein if R³ is substituted then R³ is substituted with one or more groups selected from halogen, —CN, and C₁-C₄fluoroalkyl;
R⁵ is hydrogen or R⁷;
R⁷ is substituted or unsubstituted C₁-C₆alkyl, alkyl-(substituted or unsubstituted heterocycloalkyl),
   (CH₂CH₂O)ₙ—R¹¹, or —(C(R¹⁰)₂)ₚ—OR¹¹;
each R⁹ is independently selected from hydrogen and C₁-C₆alkyl;
each R¹⁰ is independently selected from hydrogen and C₁-C₆alkyl;
R¹¹ is hydrogen, substituted or unsubstituted alkyl, —C(═O)R¹², or —P(═O)(OR⁹)₂;
R¹² is hydrogen, substituted or unsubstituted C₁-C₆alkyl, or substituted or unsubstituted C₃-C₁₀cycloalkyl;
n is 1, 2, 3, 4, 5, or 6; and
p is 1, 2, 3, 4, 5, or 6;
wherein substituted means that the referenced group is substituted with one or more additional groups individually and independently selected from C₁-C₆alkyl.

In some embodiments of Formula (III)
R¹ and R² are each independently selected from substituted or unsubstituted C₁-C₆alkyl;
R³ is selected from substituted or unsubstituted phenyl.

In some embodiments of Formula (III)
R¹ and R² are each independently selected from methyl, ethyl, n-propyl, iso-propyl, n-butyl, iso-butyl, tert-butyl, n-pentyl, tert-pentyl, neopentyl, isopentyl, sec-pentyl, 3-pentyl, n-hexyl, isohexyl, 3-methylpentyl, 2,3-dimethylbutyl, and neohexyl.

In some embodiments of Formula (III), the compound has the following structure:

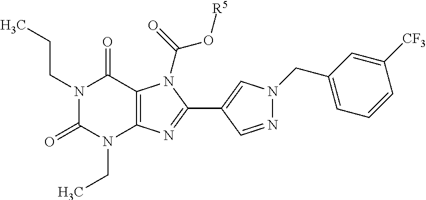

or a pharmaceutically acceptable salt or solvate thereof. In some of such embodiments,
R⁵ is R⁷;
R⁷ is C₁-C₆alkyl, —(CH₂CH₂O)ₙ—R¹¹, or —(C(R¹⁰)₂)ₚ—OR¹¹;
each R¹⁰ is independently selected from hydrogen and methyl;
R¹¹ is hydrogen, C₁-C₆alkyl, —C(═O)R¹², or —P(═O)(OR⁹)₂;
R¹² is substituted or unsubstituted C₁-C₆alkyl, or substituted or unsubstituted C₃-C₁₀cycloalkyl;
n is 1, 2, 3, 4, 5, or 6; and
p is 1, 2, 3, 4, 5, or 6;
wherein substituted means that the referenced group is substituted with one or more additional groups individually and independently selected from C₁-C₆alkyl.

In some embodiments of Formula (III), R⁷ is C₁-C₆alkyl.

In some embodiments of Formula (III), the compound has one of the following structures:

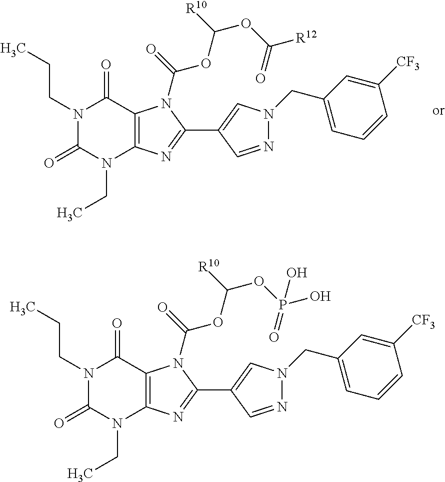

or a pharmaceutically acceptable salt or solvate thereof.

In some embodiments, a prodrug of Compound 1 is a compound represented by Formula (III) and the compound has one of the following structures:

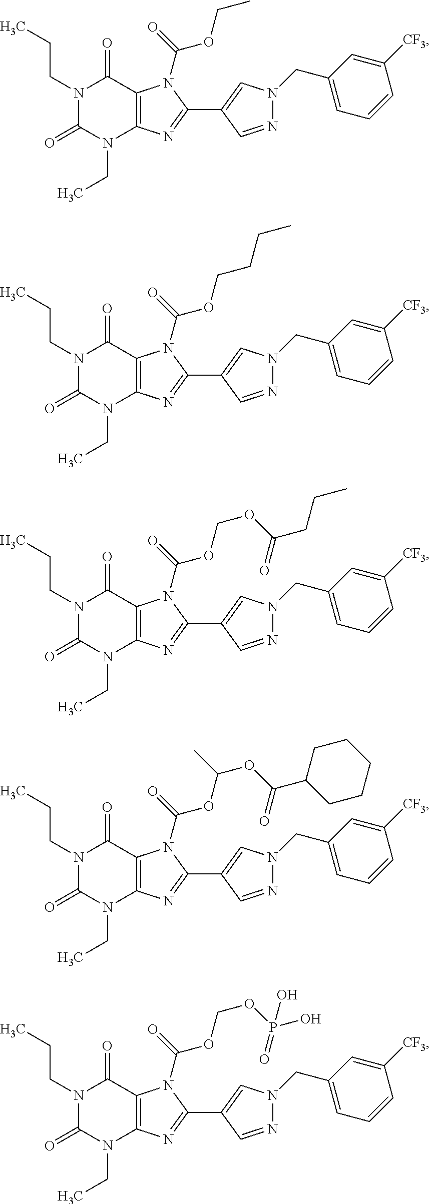 [SMILES: CCCn1c(=O)c2c(nc(-c3cnn(Cc4cccc(C(F)(F)F)c4)c3)n2C(=O)OCC)n(CC)c1=O ; CCCCOC(=O)n1c(-c2cnn(Cc3cccc(C(F)(F)F)c3)c2)nc2c1c(=O)n(CCC)c(=O)n2CC ; CCCC(=O)OCOC(=O)n1c(-c2cnn(Cc3cccc(C(F)(F)F)c3)c2)nc2c1c(=O)n(CCC)c(=O)n2CC ; CCCn1c(=O)c2c(nc(-c3cnn(Cc4cccc(C(F)(F)F)c4)c3)n2C(=O)OC(C)OC(=O)C2CCCCC2)n(CC)c1=O ; CCCn1c(=O)c2c(nc(-c3cnn(Cc4cccc(C(F)(F)F)c4)c3)n2C(=O)OCOP(=O)(O)O)n(CC)c1=O]

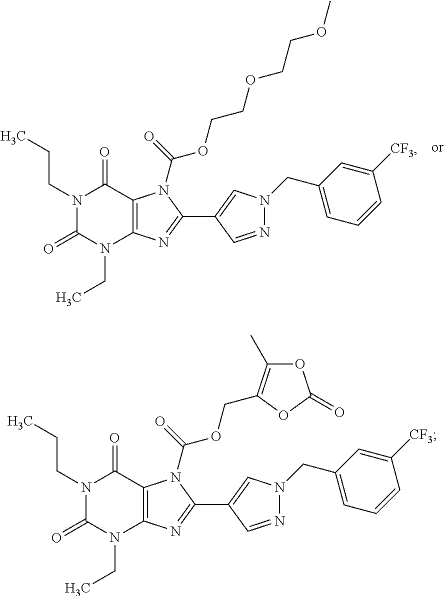 [SMILES: CCCn1c(=O)c2c(nc(-c3cnn(Cc4cccc(C(F)(F)F)c4)c3)n2C(=O)OCCOCCOC)n(CC)c1=O ; "or" ; CCCn1c(=O)c2c(nc(-c3cnn(Cc4cccc(C(F)(F)F)c4)c3)n2C(=O)OCc2oc(=O)oc2C)n(CC)c1=O]

or a pharmaceutically acceptable salt thereof.

In some embodiments, a prodrug of Compound 1 is a compound of structure:

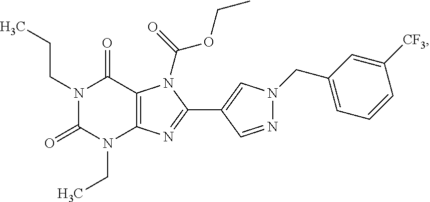 [SMILES: CCCn1c(=O)c2c(nc(-c3cnn(Cc4cccc(C(F)(F)F)c4)c3)n2C(=O)OCC)n(CC)c1=O]

or a pharmaceutically acceptable salt thereof.

In some embodiments, a prodrug of Compound 1 is a compound of structure:

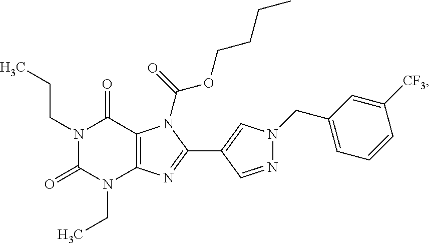 [SMILES: CCCCOC(=O)n1c(-c2cnn(Cc3cccc(C(F)(F)F)c3)c2)nc2c1c(=O)n(CCC)c(=O)n2CC]

or a pharmaceutically acceptable salt thereof.

In some embodiments, a prodrug of Compound 1 is a compound of structure:

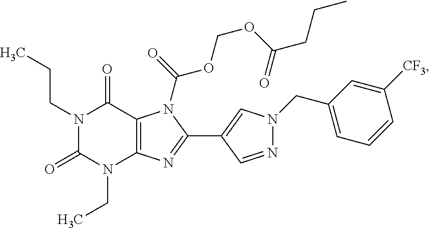 [SMILES: CCCC(=O)OCOC(=O)n1c(-c2cnn(Cc3cccc(C(F)(F)F)c3)c2)nc2c1c(=O)n(CCC)c(=O)n2CC]

or a pharmaceutically acceptable salt thereof.

In some embodiments, a prodrug of Compound 1 is a compound of structure:

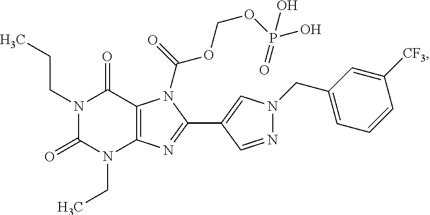 [SMILES: CCCn1c(=O)c2c(nc(-c3cnn(Cc4cccc(C(F)(F)F)c4)c3)n2C(=O)OCOP(=O)(O)O)n(CC)c1=O]

or a pharmaceutically acceptable salt thereof.

In some embodiments, a prodrug of Compound 1 is a compound of structure:

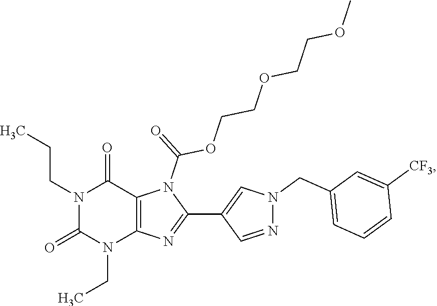 [SMILES: CCCn1c(=O)c2c(nc(-c3cnn(Cc4cccc(C(F)(F)F)c4)c3)n2C(=O)OCCOCCOC)n(CC)c1=O]

or a pharmaceutically acceptable salt thereof.

In some embodiments, the compound (e.g., a prodrug of Compound 1) has the following structure:

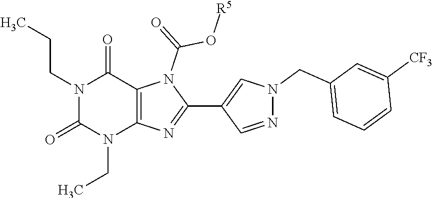

or a pharmaceutically acceptable salt or solvate thereof.

In some embodiments, R⁵ is R⁷; R⁷ is C₁-C₆alkyl, substituted or unsubstituted C₁-C₆heteroalkyl, substituted or unsubstituted monocyclic C₃-C₈cycloalkyl, substituted or unsubstituted bicyclic C₅-C₁₀cycloalkyl, substituted or unsubstituted monocyclic C₂-C₈heterocycloalkyl, substituted or unsubstituted bicyclic C₅-C₁₀heterocycloalkyl, substituted or unsubstituted phenyl, substituted or unsubstituted monocyclic heteroaryl, —CH₂-(substituted or unsubstituted phenyl), —CH₂-(substituted or unsubstituted heteroaryl), —CH₂-(substituted or unsubstituted C₂-C₈heterocycloalkyl), —CH(R¹⁰)O—R¹¹, —(CH₂CH₂O)ₙ—R¹¹, or —(C(R¹⁰)₂)ₚ—OR¹¹; each R¹⁰ is independently selected from hydrogen and methyl; R¹¹ is hydrogen, C₁-C₆alkyl, substituted or unsubstituted C₁-C₆heteroalkyl, substituted or unsubstituted C₂-C₁₀heterocycloalkyl, —C(═O)R¹², —C(═O)—OR¹², —C(═O)N(R¹²)(R′), —C(═O)—SR¹², or —P(═O)(OR⁹)₂.

In some embodiments, R⁷ is C₁-C₆alkyl, substituted or unsubstituted C₁-C₆heteroalkyl, —CH₂-(substituted or unsubstituted phenyl), —CH₂-(substituted or unsubstituted heteroaryl), —CH₂-(substituted or unsubstituted C₂-C₈heterocycloalkyl), —CH(R¹⁰)O—R¹¹, or —(CH₂CH₂O)ₙ—R¹¹; R¹⁰ is hydrogen and methyl; R¹¹ is hydrogen, C₁-C₆alkyl, substituted or unsubstituted C₁-C₆heteroalkyl, substituted or unsubstituted C₂-C₁₀heterocycloalkyl, —C(═O)R¹², —C(═O)—OR¹², —C(═O)N(R¹²)(R′), —C(═O)—SR¹², or —P(═O)(OH)₂.

In some embodiments, the compound (e.g., a prodrug of Compound 1) has one of the following structures:

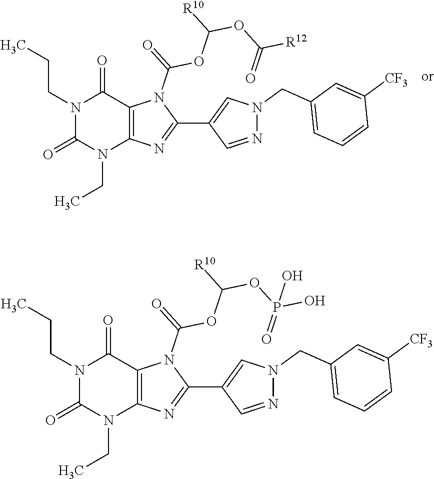

or a pharmaceutically acceptable salt or solvate thereof.

In some embodiments, the compound (e.g., a prodrug of Compound 1) has one of the following structures:

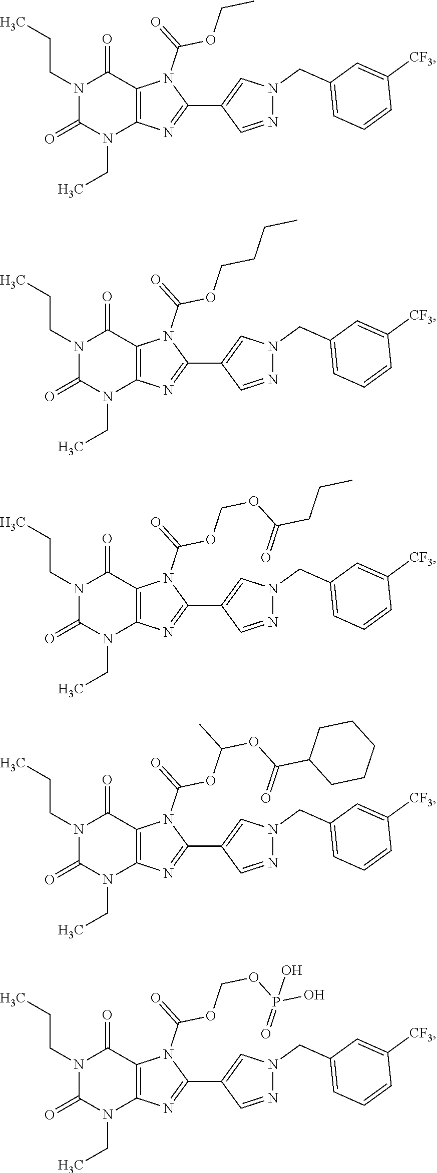 [SMILES: CCCn1c(=O)c2c(nc(-c3cnn(Cc4cccc(C(F)(F)F)c4)c3)n2C(=O)OCC)n(CC)c1=O ; CCCCOC(=O)n1c(-c2cnn(Cc3cccc(C(F)(F)F)c3)c2)nc2c1c(=O)n(CCC)c(=O)n2CC ; CCCC(=O)OCOC(=O)n1c(-c2cnn(Cc3cccc(C(F)(F)F)c3)c2)nc2c1c(=O)n(CCC)c(=O)n2CC ; CCCn1c(=O)c2c(nc(-c3cnn(Cc4cccc(C(F)(F)F)c4)c3)n2C(=O)OC(C)OC(=O)C2CCCCC2)n(CC)c1=O ; CCCn1c(=O)c2c(nc(-c3cnn(Cc4cccc(C(F)(F)F)c4)c3)n2C(=O)OCOP(=O)(O)O)n(CC)c1=O]

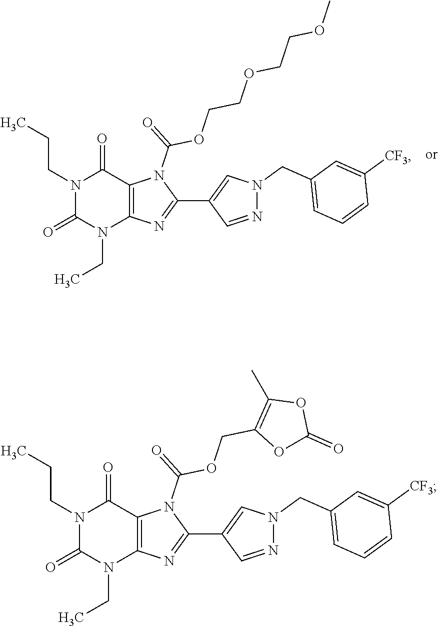 [SMILES: CCCn1c(=O)c2c(nc(-c3cnn(Cc4cccc(C(F)(F)F)c4)c3)n2C(=O)OCCOCCOC)n(CC)c1=O ; "or" ; CCCn1c(=O)c2c(nc(-c3cnn(Cc4cccc(C(F)(F)F)c4)c3)n2C(=O)OCc2oc(=O)oc2C)n(CC)c1=O]

or a pharmaceutically acceptable salt or solvate thereof.

In some embodiments, the compound (e.g., a prodrug of Compound 1) has the following structure of Formula (I):

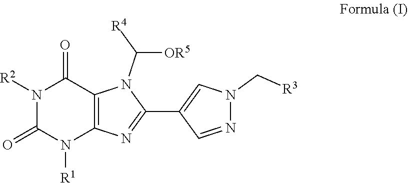

or a pharmaceutically acceptable salt or solvate thereof.

In some embodiments, R¹ and R² are each independently selected from substituted or unsubstituted C₁-C₆alkyl; R³ is selected from substituted or unsubstituted phenyl.

In some embodiments, R¹ is ethyl; R² is n-propyl; and R³ is 3-(trifluoromethyl)phenyl.

In some embodiments, the compound (e.g., a prodrug of Compound 1) has the following structure:

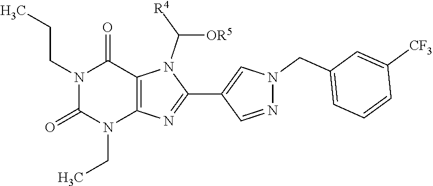

or a pharmaceutically acceptable salt or solvate thereof.

In some embodiments, R⁴ is methyl or ethyl; R⁵ is hydrogen, R⁷, —C(═O)R⁷, —C(═O)—OR⁷, —C(═O)N(R⁷)(R⁸), —C(═O)—SR⁷, or —P(═O)(OR⁹)₂; R⁷ is C₁-C₆alkyl, substituted or unsubstituted C₁-C₆heteroalkyl, substituted or unsubstituted monocyclic C₃-C₈cycloalkyl, substituted or unsubstituted bicyclic C₅-C₁₀cycloalkyl, substituted or unsubstituted monocyclic C₂-C₈heterocycloalkyl, substituted or unsubstituted bicyclic C₅-C₁₀heterocycloalkyl, substituted or unsubstituted phenyl, substituted or unsubstituted monocyclic heteroaryl, —CH₂-(substituted or unsubstituted phenyl), —CH₂-(substituted or unsubstituted heteroaryl), —CH₂-(substituted or unsubstituted C₂-C₈heterocycloalkyl), —CH(R¹⁰)O—R¹¹, —(CH₂CH₂O)ₙ—R¹¹, or —(C(R¹⁰)₂)ₚ—OR¹¹; each R¹⁰ is independently selected from hydrogen and methyl; R¹¹ is hydrogen, C₁-C₆alkyl, substituted or unsubstituted C₁-C₆heteroalkyl, substituted or unsubstituted C₂-C₁₀heterocycloalkyl, —C(═O)R¹², —C(═O)—OR¹², —C(═O)N(R¹²)(RI), —C(═O)—SR¹², or —P(═O)(OR⁹)₂.

In some embodiments, R⁵ is R⁷, —C(═O)R⁷, —C(═O)—OR⁷, —C(═O)N(R⁷)(R⁸), —C(═O)—SR⁷, or —P(═O)(OH)₂; R⁷ is C₁-C₆alkyl, substituted or unsubstituted C₁-C₆heteroalkyl, substituted or unsubstituted cyclohexyl, substituted or unsubstituted cyclopentyl, substituted or unsubstituted bicyclo[1.1.1]pentanyl, substituted or unsubstituted bicyclo[2.2.1]heptanyl, substituted or unsubstituted bicyclo[2.2.2]octanyl, substituted or unsubstituted bicyclo[3.2.1]octanyl, substituted or unsubstituted bicyclo[3.3.0]octanyl, substituted or unsubstituted bicyclo[4.3.0]nonanyl, or substituted or unsubstituted decalinyl, substituted or unsubstituted oxetanyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, substituted or unsubstituted thiomorpholinyl, substituted or unsubstituted phenyl, substituted or unsubstituted monocyclic heteroaryl, —CH₂-(substituted or unsubstituted phenyl), —CH₂-(substituted or unsubstituted heteroaryl), —CH₂-(substituted or unsubstituted C₂-C₈heterocycloalkyl), —CH(R¹⁰)O—R¹¹, —(CH₂CH₂O)ₙ—R¹¹, or —(C(R¹⁰)₂)ₚ—OR¹¹; each R¹⁰ is independently selected from hydrogen and methyl; R¹¹ is hydrogen, C₁-C₆alkyl, substituted or unsubstituted C₁-C₆heteroalkyl, substituted or unsubstituted C₂-C₁₀heterocycloalkyl, —C(═O)R¹², —C(═O)—OR¹², —C(═O)N(R¹²)(R⁸), —C(═O)—SR¹², or —P(═O)(OR⁹)₂.

In some embodiments, the compound (e.g., a prodrug of Compound 1) has one of the following structures:

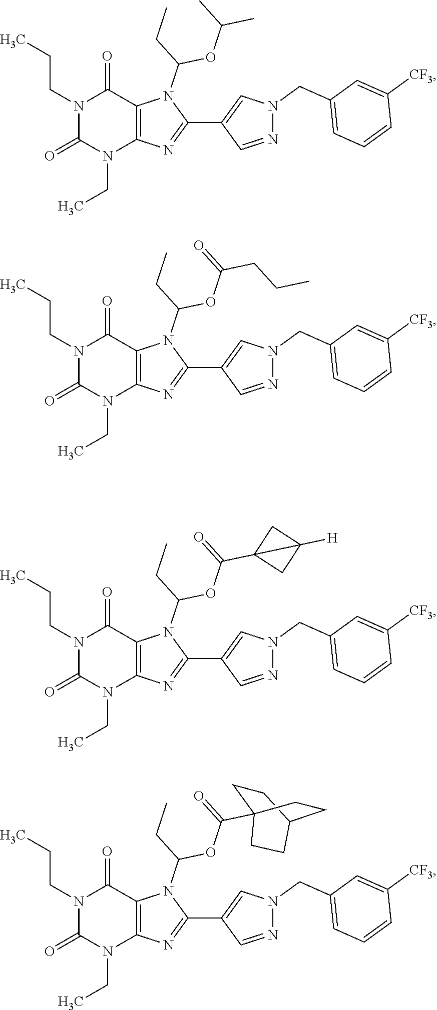 [SMILES: CCCn1c(=O)c2c(nc(-c3cnn(Cc4cccc(C(F)(F)F)c4)c3)n2C(CC)OC(C)C)n(CC)c1=O ; CCCC(=O)OC(CC)n1c(-c2cnn(Cc3cccc(C(F)(F)F)c3)c2)nc2c1c(=O)n(CCC)c(=O)n2CC ; CCCn1c(=O)c2c(nc(-c3cnn(Cc4cccc(C(F)(F)F)c4)c3)n2C(CC)OC(=O)C23CC2C3)n(CC)c1=O ; CCCn1c(=O)c2c(nc(-c3cnn(Cc4cccc(C(F)(F)F)c4)c3)n2C(CC)OC(=O)C23CCC(CC2)CC3)n(CC)c1=O]

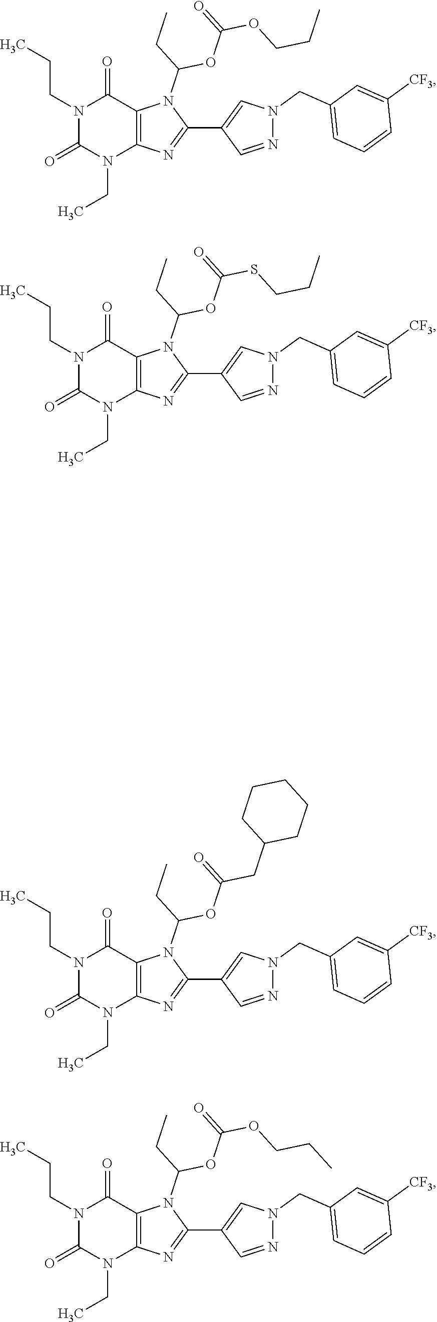 [SMILES: CCCOC(=O)OC(CC)n1c(-c2cnn(Cc3cccc(C(F)(F)F)c3)c2)nc2c1c(=O)n(CCC)c(=O)n2CC ; CCCSC(=O)OC(CC)n1c(-c2cnn(Cc3cccc(C(F)(F)F)c3)c2)nc2c1c(=O)n(CCC)c(=O)n2CC ; CCCn1c(=O)c2c(nc(-c3cnn(Cc4cccc(C(F)(F)F)c4)c3)n2C(CC)OC(=O)CC2CCCCC2)n(CC)c1=O ; CCCOC(=O)OC(CC)n1c(-c2cnn(Cc3cccc(C(F)(F)F)c3)c2)nc2c1c(=O)n(CCC)c(=O)n2CC]

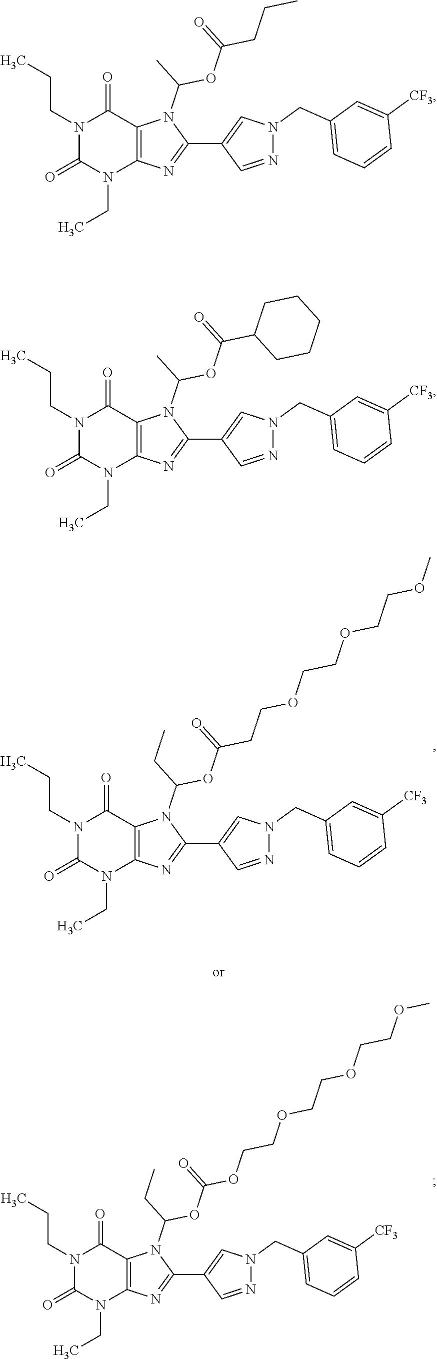

or a pharmaceutically acceptable salt or solvate thereof.

In some embodiments, the compound (e.g., a prodrug of Compound 1) has the following structure of Formula (II):

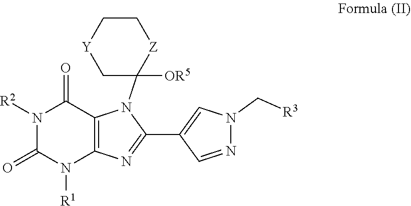

wherein:
Y is selected from —CH₂—, O, S, —NR¹⁵—, and —S(O)₂—;
Z is O or S;
or a pharmaceutically acceptable salt or solvate thereof.

In some embodiments, R¹ and R² are each independently selected from substituted or unsubstituted C₁-C₆alkyl; R³ is selected from substituted or unsubstituted phenyl.

In some embodiments, R¹ is ethyl; R² is n-propyl; and R³ is 3-(trifluoromethyl)phenyl.

In some embodiments, the compound (e.g., a prodrug of Compound 1) has the following structure:

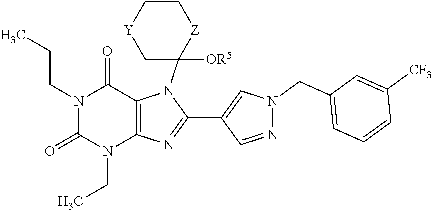

or a pharmaceutically acceptable salt or solvate thereof.

In some embodiments, the compound (e.g., a prodrug of Compound 1) has the following structure:

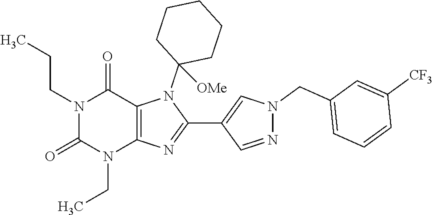 [SMILES: CCCn1c(=O)c2c(nc(-c3cnn(Cc4cccc(C(F)(F)F)c4)c3)n2C2(OC)CCCCC2)n(CC)c1=O]

or a pharmaceutically acceptable salt or solvate thereof.

In some embodiments, the compound (e.g., a prodrug of Compound 1) has the following structure of Formula (IIa):

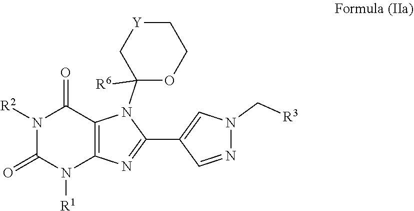

wherein:
Y is selected from —CH₂—, O, S, —NR¹⁵—, and —S(O)₂—;
or a pharmaceutically acceptable salt or solvate thereof.

In some embodiments, R¹ and R² are each independently selected from substituted or unsubstituted C₁-C₆alkyl; R³ is selected from substituted or unsubstituted phenyl.

In some embodiments, substituted means that the referenced group is substituted with one or more additional groups individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, substituted means that the referenced group is substituted with one or more additional groups individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, and heterocycloalkyl. In yet other embodiments, substituted means that the referenced group is substituted with one or more additional groups individually and independently selected from halogen, —CN, —NH₂, —NH(alkyl), —N(alkyl)₂, —OH, —CO₂H, —CO₂alkyl, —C(═O)NH₂, —C(═O)NIH(alkyl), —C(═O)N(alkyl)₂, —S(═O)₂NH₂, —S(═O)₂NH(alkyl), —S(═O)₂N(alkyl)₂, alkyl, fluoroalkyl, alkoxy, and fluoroalkoxy.

In some embodiments, R¹ is ethyl; R² is n-propyl; and R³ is 3-(trifluoromethyl)phenyl.

In some embodiments, the compound (e.g., a prodrug of Compound 1) has the following structure:

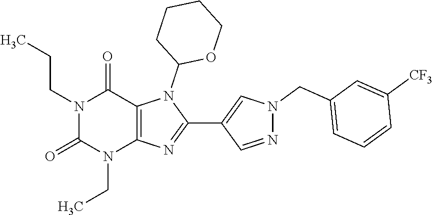 [SMILES: CCCn1c(=O)c2c(nc(-c3cnn(Cc4cccc(C(F)(F)F)c4)c3)n2C2CCCCO2)n(CC)c1=O]

or a pharmaceutically acceptable salt or solvate thereof.

In some embodiments, compounds of Formula (A) (e.g., prodrugs of Compound 1) include those described in Table 1.

TABLE 1

| Compound | Structure | Name |
|---|---|---|
| A | 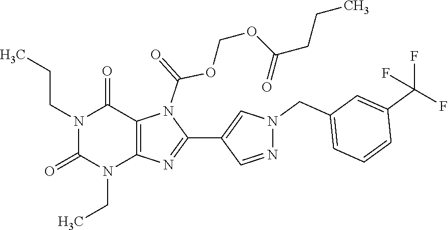 [SMILES: CCCC(=O)OCOC(=O)n1c(-c2cnn(Cc3cccc(C(F)(F)F)c3)c2)nc2c1c(=O)n(CCC)c(=O)n2CC] | (butyryloxy)methyl 3-ethyl-2,6- dioxo-1-propyl-8-(1-(3- (trifluoromethyl)benzyl)-1H- pyrazol-4-yl)-1,2,3,6-tetrahydro- 7H-purine-7-carboxylate |
| B | 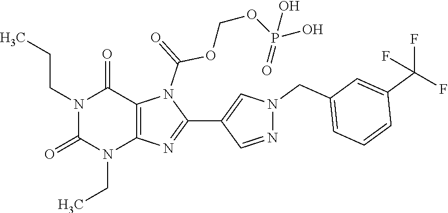 [SMILES: CCCn1c(=O)c2c(nc(-c3cnn(Cc4cccc(C(F)(F)F)c4)c3)n2C(=O)OCOP(=O)(O)O)n(CC)c1=O] | (phosphonooxy)methyl 3-ethyl- 2,6-dioxo-1-propyl-8-(1-(3- (trifluoromethyl)benzyl)-1H- pyrazol-4-yl)-1,2,3,6-tetrahydro- 7H-purine-7-carboxylate |
| F | 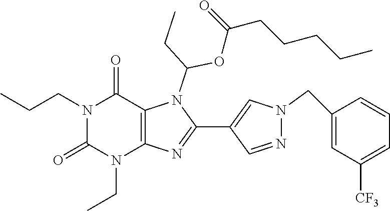 [SMILES: CCCCCC(=O)OC(CC)n1c(-c2cnn(Cc3cccc(C(F)(F)F)c3)c2)nc2c1c(=O)n(CCC)c(=O)n2CC] | 1-(3-ethyl-2,6-dioxo-1-propyl-8- (1-(3-(trifluoromethyl)benzyl)-1H- pyrazol-4-yl)-1,2,3,6-tetrahydro- 7H-purin-7-yl)propyl hexanoate |
| G | 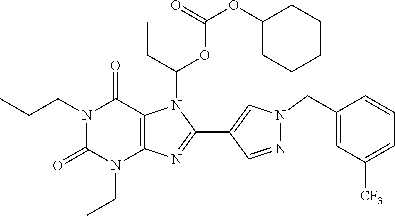 [SMILES: CCCn1c(=O)c2c(nc(-c3cnn(Cc4cccc(C(F)(F)F)c4)c3)n2C(CC)OC(=O)OC2CCCCC2)n(CC)c1=O] | cyclohexyl (1-(3-ethyl-2,6-dioxo- 1-propyl-8-(1-(3- (trifluoromethyl)benzyl)-1H- pyrazol-4-yl)-1,2,3,6-tetrahydro- 7H-purin-7-yl)propyl)carbonate |
| H | 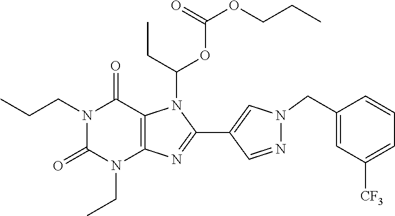 [SMILES: CCCOC(=O)OC(CC)n1c(-c2cnn(Cc3cccc(C(F)(F)F)c3)c2)nc2c1c(=O)n(CCC)c(=O)n2CC] | 1-(3-ethyl-2,6-dioxo-1-propyl-8- (1-(3-(trifluoromethyl)benzyl)-1H- pyrazol-4-yl)-1,2,3,6-tetrahydro- 7H-purin-7-yl)propyl propyl carbonate |
| I | 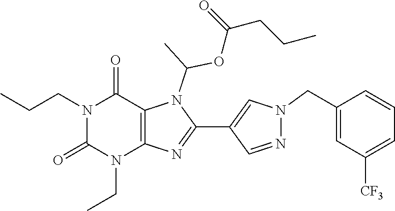 [SMILES: CCCC(=O)OC(C)n1c(-c2cnn(Cc3cccc(C(F)(F)F)c3)c2)nc2c1c(=O)n(CCC)c(=O)n2CC] | 1-(3-ethyl-2,6-dioxo-1-propyl-8- (1-(3-(trifluoromethyl)benzyl)-1H- pyrazol-4-yl)-1,2,3,6-tetrahydro- 7H-purin-7-yl)ethyl butyrate |
| K | 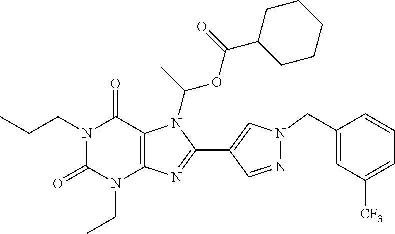 [SMILES: CCCn1c(=O)c2c(nc(-c3cnn(Cc4cccc(C(F)(F)F)c4)c3)n2C(C)OC(=O)C2CCCCC2)n(CC)c1=O] | 1-(3-ethyl-2,6-dioxo-1-propyl-8- (1-(3-(trifluoromethyl)benzyl)-1H- pyrazol-4-yl)-1,2,3,6-tetrahydro- 7H-purin-7-yl)ethyl cyclohexanecarboxylate |
| M | 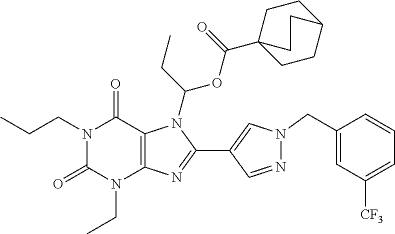 [SMILES: CCCn1c(=O)c2c(nc(-c3cnn(Cc4cccc(C(F)(F)F)c4)c3)n2C(CC)OC(=O)C23CCC(CC2)CC3)n(CC)c1=O] | 1-(3-ethyl-2,6-dioxo-1-propyl-8- (1-(3-(trifluoromethyl)benzyl)-1H- pyrazol-4-yl)-1,2,3,6-tetrahydro- 7H-purin-7-yl)propyl bicyclo[2.2.2]octane-1-carboxylate |
| O | 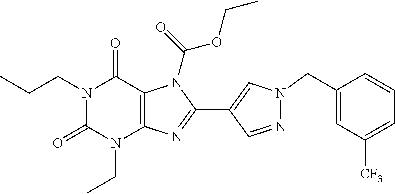 [SMILES: CCCn1c(=O)c2c(nc(-c3cnn(Cc4cccc(C(F)(F)F)c4)c3)n2C(=O)OCC)n(CC)c1=O] | ethyl 3-ethyl-2,6-dioxo-1-propyl- 8-(1-(3-(trifluoromethyl)benzyl)- 1H-pyrazol-4-yl)-1,2,3,6- tetrahydro-7H-purine-7- carboxylate |
| P | 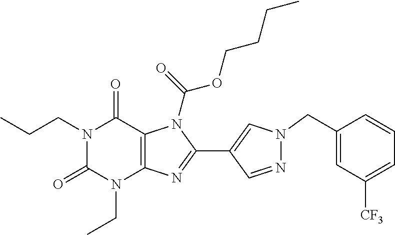 [SMILES: CCCCOC(=O)n1c(-c2cnn(Cc3cccc(C(F)(F)F)c3)c2)nc2c1c(=O)n(CCC)c(=O)n2CC] | butyl 3-ethyl-2,6-dioxo-1-propyl- 8-(1-(3-(trifluoromethyl)benzyl)- 1H-pyrazol-4-yl)-1,2,3,6- tetrahydro-7H-purine-7- carboxylate |
| Q | 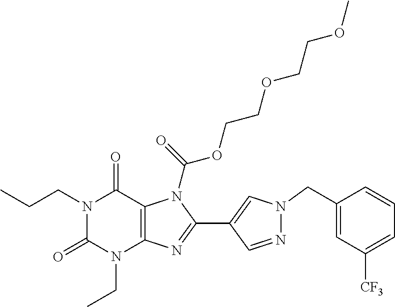 [SMILES: CCCn1c(=O)c2c(nc(-c3cnn(Cc4cccc(C(F)(F)F)c4)c3)n2C(=O)OCCOCCOC)n(CC)c1=O] | 2-(2-methoxyethoxy)ethyl 3-ethyl- 2,6-dioxo-1-propyl-8-(1-(3- (trifluoromethyl)benzyl)-1H- pyrazol-4-yl)-1,2,3,6-tetrahydro- 7H-purine-7-carboxylate |
| S | 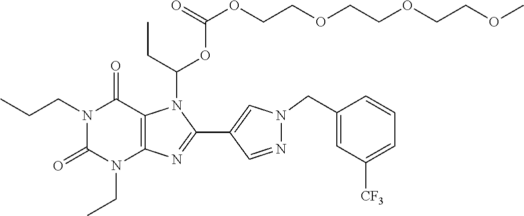 [SMILES: CCCn1c(=O)c2c(nc(-c3cnn(Cc4cccc(C(F)(F)F)c4)c3)n2C(CC)OC(=O)OCCOCCOCCOC)n(CC)c1=O] | 1-(3-ethyl-2,6-dioxo-1-propyl-8- (1-(3-(trifluoromethyl)benzyl)-1H- pyrazol-4-yl)-1,2,3,6-tetrahydro- 7H-purin-7-yl)propyl (2-(2-(2- methoxyethoxy)ethoxy)ethyl) carbonate |
| T | 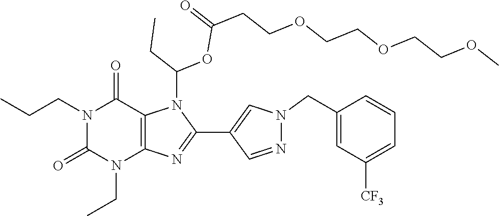 [SMILES: CCCn1c(=O)c2c(nc(-c3cnn(Cc4cccc(C(F)(F)F)c4)c3)n2C(CC)OC(=O)CCOCCOCCOC)n(CC)c1=O] | 1-(3-ethyl-2,6-dioxo-1-propyl-8- (1-(3-(trifluoromethyl)benzyl)-1H- pyrazol-4-yl)-1,2,3,6-tetrahydro- 7H-purin-7-yl)propyl 3-(2-(2- methoxyethoxy)ethoxy)propanoate |

In another aspect, described herein is a compound (e.g., a prodrug of Compound 1) represented by Formula (B):

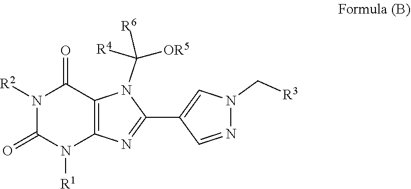

or a pharmaceutically acceptable salt or solvate thereof, wherein:
R¹ and R² are each independently selected from hydrogen, and substituted or unsubstituted alkyl;
R³ is selected from substituted or unsubstituted phenyl, and substituted or unsubstituted heteroaryl, wherein if R³ is substituted then R³ is substituted with one or more groups selected from halogen, —CN, —OH, C₁-C₄alkyl, C₂-C₄alkenyl, C₂-C₄alkynyl, C₁-C₄alkoxy, C₁-C₄fluoroalkyl, C₁-C₄fluoroalkoxy, and substituted or unsubstituted C₁-C₄heteroalkyl;
R⁴ is hydrogen or substituted or unsubstituted alkyl;
R⁶ is hydrogen or substituted or unsubstituted alkyl;
or R⁴ and R⁶ are taken together with the carbon atom to which they are attached to form a carbonyl (C═O);
or R⁴ and R⁶ are taken together with the carbon atom to which they are attached to form a ring that is a substituted or unsubstituted C₃-C₁₀cycloalkyl, or substituted or unsubstituted C₂-C₁₀heterocycloalkyl, wherein if the ring is substituted then it is substituted with one or more R⁵;
   R¹⁵ is hydrogen, substituted or unsubstituted alkyl, substituted or unsubstituted heteroalkyl, substituted or unsubstituted phenyl, substituted or unsubstituted heteroaryl, -alkyl-(substituted or unsubstituted phenyl), -alkyl-(substituted or unsubstituted heteroaryl), —C(═O)R¹⁶, —C(═O)—OR¹⁶, —C(═O)N(R¹⁶)₂;
   each R¹⁶ is independently selected from hydrogen and substituted or unsubstituted alkyl;
R⁵ is substituted or unsubstituted C₃-C₁₀cycloalkyl, substituted or unsubstituted C₂-C₁₀heterocycloalkyl, substituted or unsubstituted phenyl, substituted or unsubstituted heteroaryl, -alkyl-(substituted or unsubstituted phenyl), -alkyl-(substituted or unsubstituted heteroaryl), -alkyl-(substituted or unsubstituted cycloalkyl),-alkyl-(substituted or unsubstituted heterocycloalkyl), —(C(R¹⁰)₂O)ₘ—R¹¹, —C(═O)—(C(R¹⁰)₂O)ₘ—R¹¹, —C(═O)—(CH₂CH₂O)ₙ—R¹¹, —C(═O)—R^{a} or —C(═O)—OR⁷;
R^{a} is substituted or unsubstituted bicyclic cycloalkyl, substituted or unsubstituted bicyclic heterocycloalkyl, substituted or unsubstituted bicyclic heteroaryl, (substituted or unsubstituted heterocycloalkyl containing at least one O atom in the ring), substituted or unsubstituted azetidinyl, substituted or unsubstituted piperidinyl, substituted or unsubstituted azepinyl, substituted or unsubstituted 5-membered heteroaryl, substituted or unsubstituted pyridin-2-yl, substituted or unsubstituted pyridin-4-yl, substituted or unsubstituted pyrimidinyl, substituted or unsubstituted pyrazinyl, substituted or unsubstituted pyridazinyl, substituted or unsubstituted triazinyl;
or R⁴ and R⁵ are taken together with the atoms to which they are attached to form a substituted or unsubstituted C₂-C₁₀heterocycloalkyl;
R⁷ is substituted or unsubstituted alkyl, substituted or unsubstituted heteroalkyl, substituted or unsubstituted C₃-C₁₀cycloalkyl, substituted or unsubstituted C₂-C₁₀heterocycloalkyl, substituted or unsubstituted phenyl, substituted or unsubstituted heteroaryl, -alkyl-(substituted or unsubstituted phenyl), -alkyl-(substituted or unsubstituted heteroaryl), -alkyl-(substituted or unsubstituted cycloalkyl),-alkyl-(substituted or unsubstituted heterocycloalkyl), —(C(R¹⁰)₂O)ₘ—R¹¹, —(CH₂CH₂O)ₙ—R¹¹, or —(C(R¹⁰)₂)ₚ—OR¹¹;
each R⁹ is independently selected from hydrogen and alkyl;
each R¹⁰ is independently selected from hydrogen and alkyl;
R¹¹ is hydrogen, substituted or unsubstituted alkyl, substituted or unsubstituted heteroalkyl, substituted or unsubstituted C₂-C₁₀heterocycloalkyl, —C(═O)R¹², —C(═O)—OR¹², —C(═O)N(R¹²)(RI), —C(═O)—SR¹², or —P(═O)(OR⁹)₂;
R¹² is hydrogen, substituted or unsubstituted alkyl, substituted or unsubstituted heteroalkyl, substituted or unsubstituted C₃-C₁₀cycloalkyl, substituted or unsubstituted C₂-C₁₀heterocycloalkyl, substituted or unsubstituted phenyl, substituted or unsubstituted heteroaryl, -alkyl-(substituted or unsubstituted phenyl), or -alkyl-(substituted or unsubstituted heteroaryl);
m is 1, 2, 3, 4, 5, or 6;
n is 1, 2, 3, 4, 5, or 6.
p is 1, 2, 3, 4, 5, or 6;
wherein substituted means that the referenced group is substituted with one or more additional groups individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone.

In some embodiments, m is 1, 2, 3, 4, 5, or 6. In some embodiments, m is 1, 2, 3, 4, or 5. In some embodiments, m is 1, 2, 3, or 4. In some embodiments, m is 1, 2, or 3. In some embodiments, m is 1 or 2. In some embodiments, m is 1. In some embodiments, m is 2, 3, 4, 5, or 6.

In some embodiments, n is 1, 2, 3, 4, 5, or 6. In some embodiments, n is 1, 2, 3, 4, or 5. In some embodiments, n is 1, 2, 3, or 4. In some embodiments, n is 1, 2, or 3. In some embodiments, n is 1 or 2. In some embodiments, n is 2 or 3. In some embodiments, n is 1. In some embodiments, n is 2. In some embodiments, n is 3. In some embodiments, n is 2, 3, 4, 5, or 6.

In some embodiments, p is 1, 2, 3, 4, 5, or 6. In some embodiments, p is 1, 2, 3, 4, or 5. In some embodiments, p is 1, 2, 3, or 4. In some embodiments, p is 1, 2, or 3. In some embodiments, p is 1 or 2. In some embodiments, p is 1. In some embodiments, p is 2, 3, 4, 5, or 6.

In some embodiments, a prodrug of Compound 1 has the following structure

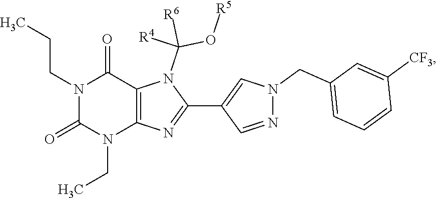

or a pharmaceutically acceptable salt or solvate thereof.

In some embodiments, R⁴ is hydrogen; R⁶ is hydrogen; R⁵ is substituted or unsubstituted C₃-C₁₀cycloalkyl, substituted or unsubstituted C₂-C₁₀heterocycloalkyl, substituted or unsubstituted phenyl, substituted or unsubstituted heteroaryl, -alkyl-(substituted or unsubstituted phenyl), -alkyl-(substituted or unsubstituted heteroaryl), -alkyl-(substituted or unsubstituted cycloalkyl),-alkyl-(substituted or unsubstituted heterocycloalkyl), —(C(R¹⁰)₂O)ₘ—R¹¹, —C(═O)—(C(R¹⁰)₂O)ₘ—R¹¹, —C(═O)—(CH₂CH₂O)ₙ—R¹¹, —C(═O)—R^{a} or —C(═O)—OR⁷.

In some embodiments, R¹ and R² are each independently selected from substituted or unsubstituted C₁-C₆alkyl. In some embodiments, R¹ and R² are each independently selected from unsubstituted C₁-C₃alkyl. In some embodiments, R¹ is ethyl. In some embodiments, R² is n-propyl. In some embodiments, R¹ is ethyl and R² is n-propyl.

In some embodiments, R³ is selected from substituted or unsubstituted phenyl. In some embodiments, R³ is substituted phenyl. In some embodiments, R³ is phenyl substituted by one or more groups independently selected from halogen, C₁-C₄ alkyl, or C₁-C₄ fluoroalkyl. In some embodiments, R³ is phenyl substituted by one or more groups independently selected from C₁-C₄ fluoroalkyl. In some embodiments, R³ is selected from phenyl substituted with one, two, or three —CF₃ substituents. In some embodiments, R³ is selected from phenyl substituted with one —CF₃ substituent. In some embodiments, R³ is

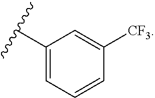 [SMILES: *c1cccc(C(F)(F)F)c1]

In some embodiments, R¹ and R² are each independently selected from substituted or unsubstituted C₁-C₆alkyl; R³ is selected from substituted or unsubstituted phenyl.

In some embodiments, R¹ and R² are each independently selected from substituted or unsubstituted C₁-C₆alkyl; R³ is selected from substituted or unsubstituted phenyl.

In some embodiments, R¹ and R² are each independently selected from methyl, ethyl, n-propyl, iso-propyl, n-butyl, iso-butyl, tert-butyl, n-pentyl, tert-pentyl, neopentyl, isopentyl, sec-pentyl, 3-pentyl, n-hexyl, isohexyl, 3-methylpentyl, 2,3-dimethylbutyl, and neohexyl.

In some embodiments, R¹ is ethyl; R² is n-propyl; and R³ is 3-(trifluoromethyl)phenyl. In some embodiments, the compound (e.g., a prodrug of Compound 1) has the following structure:

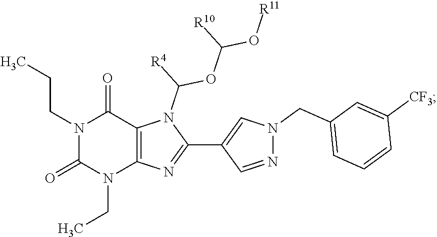

or a pharmaceutically acceptable salt or solvate thereof.

In some embodiments, R¹¹ is hydrogen, substituted or unsubstituted alkyl, —C(═O)R¹², —C(═O)—OR¹², —C(═O)N(R¹²)(R⁸), or —P(═O)(OR⁹)₂. In some embodiments, R¹¹ is substituted or unsubstituted alkyl, —C(═O)R¹², —C(═O)—OR¹², or —P(═O)(OR⁹)₂. In some embodiments, R¹¹ is —C(═O)R¹² or —P(═O)(OR⁹)₂. In some embodiments, R¹¹ is —C(═O)R¹² or —P(═O)(OH)₂.

In some embodiments, the compound (e.g., a prodrug of Compound 1) has one of the following structures:

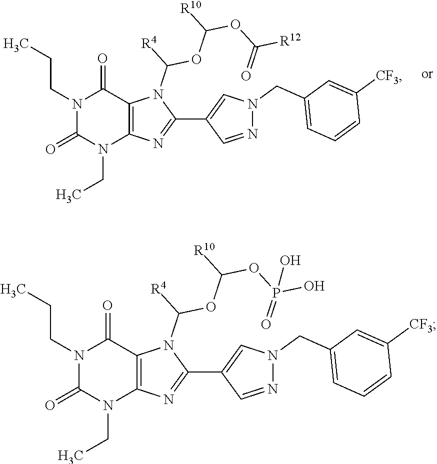

or a pharmaceutically acceptable salt or solvate thereof.

In some embodiments, R¹² is substituted or unsubstituted alkyl or substituted or unsubstituted C₃-C₁₀ cycloalkyl. In some embodiments, R¹² is unsubstituted C₁-C₆ alkyl or unsubstituted C₃-C₁₀ cycloalkyl. In some embodiments, R¹² is unsubstituted C₁-C₃ alkyl. In some embodiments, R¹² is unsubstituted C₃-C₆ cycloalkyl.

In some embodiments, the compound (e.g., a prodrug of Compound 1) has one of the following structures:

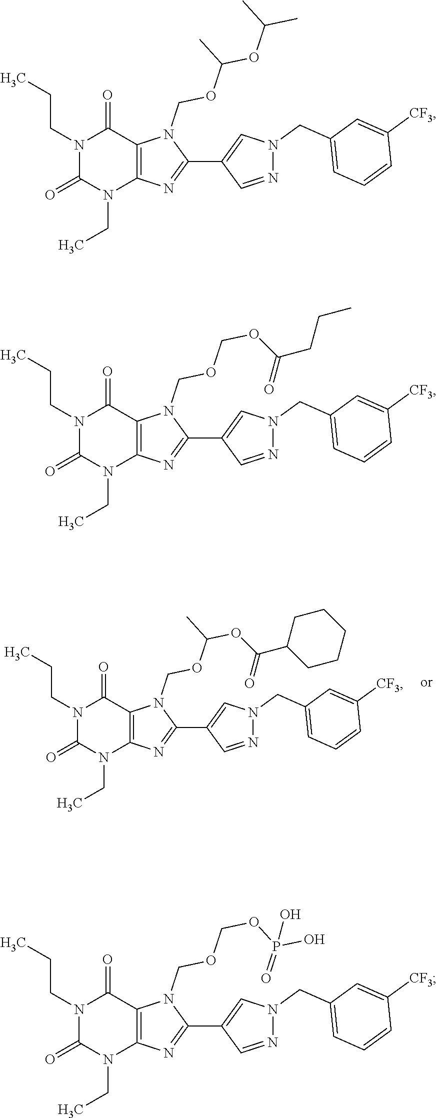 [SMILES: CCCn1c(=O)c2c(nc(-c3cnn(Cc4cccc(C(F)(F)F)c4)c3)n2COC(C)OC(C)C)n(CC)c1=O ; CCCC(=O)OCOCn1c(-c2cnn(Cc3cccc(C(F)(F)F)c3)c2)nc2c1c(=O)n(CCC)c(=O)n2CC ; CCCn1c(=O)c2c(nc(-c3cnn(Cc4cccc(C(F)(F)F)c4)c3)n2COC(C)OC(=O)C2CCCCC2)n(CC)c1=O ; "or" ; CCCn1c(=O)c2c(nc(-c3cnn(Cc4cccc(C(F)(F)F)c4)c3)n2COCOP(=O)(O)O)n(CC)c1=O]

or a pharmaceutically acceptable salt or solvate thereof.

In some embodiments, R⁵ is —C(═O)—(C(R¹⁰)₂O)ₘ—R¹¹, —C(═O)—(CH₂CH₂O)ₙ—R¹¹, —C(═O)—R^{a} or —C(═O)—OR⁷.

In some embodiments, the compound (e.g., a prodrug of Compound 1) has the following structure:

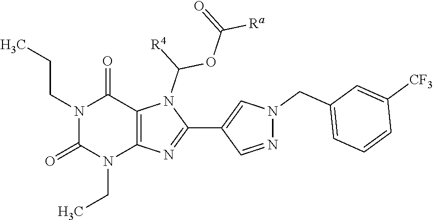

or a pharmaceutically acceptable salt or solvate thereof.

In some embodiments, R^{a} is substituted or unsubstituted bicyclic cycloalkyl that is a fused bicyclic cycloalkyl, bridged bicyclic cycloalkyl, or spiro bicyclic cycloalkyl; or R^{a} is substituted or unsubstituted bicyclic heterocycloalkyl that is a fused bicyclic heterocycloalkyl, bridged bicyclic heterocycloalkyl, or spiro bicyclic heterocycloalkyl; or R^{a} is substituted or unsubstituted bicyclic heteroaryl.

In some embodiments, R^{a} is substituted or unsubstituted bicyclo[1.1.1]pentanyl, substituted or unsubstituted bicyclo[2.2.1]heptanyl, substituted or unsubstituted bicyclo[2.2.2]octanyl, substituted or unsubstituted bicyclo[3.2.1]octanyl, substituted or unsubstituted bicyclo[3.3.0]octanyl, substituted or unsubstituted bicyclo[4.3.0]nonanyl, or substituted or unsubstituted decalinyl.

In some embodiments, the compound (e.g., a prodrug of Compound 1) has one of the following structures:

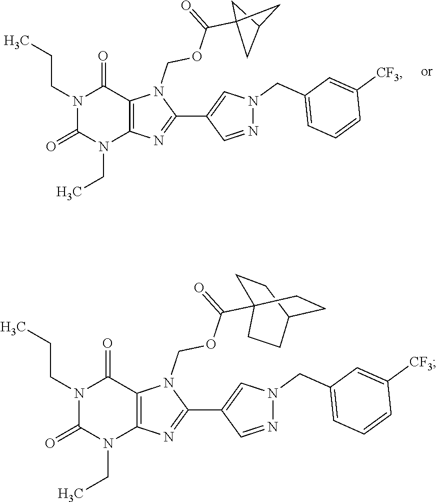 [SMILES: CCCn1c(=O)c2c(nc(-c3cnn(Cc4cccc(C(F)(F)F)c4)c3)n2COC(=O)C23CC(C2)C3)n(CC)c1=O ; "or" ; CCCn1c(=O)c2c(nc(-c3cnn(Cc4cccc(C(F)(F)F)c4)c3)n2COC(=O)C23CCC(CC2)CC3)n(CC)c1=O]

or a pharmaceutically acceptable salt or solvate thereof.

In some embodiments, R^{a} is substituted or unsubstituted heterocycloalkyl containing at least one O atom in the ring, substituted or unsubstituted azetidinyl, substituted or unsubstituted piperidinyl, substituted or unsubstituted azepinyl, substituted or unsubstituted 5-membered heteroaryl, substituted or unsubstituted pyridin-2-yl, substituted or unsubstituted pyridin-4-yl, substituted or unsubstituted pyrimidinyl, substituted or unsubstituted pyrazinyl, substituted or unsubstituted pyridazinyl, substituted or unsubstituted triazinyl. In some embodiments, R^{a} is substituted or unsubstituted tetrahydrofuranyl, substituted or unsubstituted tetrahydropyranyl, substituted or unsubstituted tetrahydrodioxanyl, substituted or unsubstituted azetidinyl, substituted or unsubstituted piperidinyl, substituted or unsubstituted azepinyl, substituted or unsubstituted pyrrolyl, substituted or unsubstituted imidazolyl, substituted or unsubstituted pyrazolyl, substituted or unsubstituted triazolyl, substituted or unsubstituted tetrazolyl, substituted or unsubstituted oxazolyl, substituted or unsubstituted isoxazolyl, substituted or unsubstituted thiazolyl, substituted or unsubstituted isothiazolyl, substituted or unsubstituted pyridin-2-yl, substituted or unsubstituted pyridin-4-yl, substituted or unsubstituted pyrimidinyl, substituted or unsubstituted pyrazinyl, substituted or unsubstituted pyridazinyl, substituted or unsubstituted triazinyl. In some embodiments, R^{a} is substituted or unsubstituted tetrahydrodioxanyl, substituted or unsubstituted azetidinyl, substituted or unsubstituted piperidinyl, substituted or unsubstituted imidazolyl, substituted or unsubstituted pyridin-2-yl, substituted or unsubstituted pyridin-4-yl, or substituted or unsubstituted pyrimidinyl.

In some embodiments, R^{a} is a substituted or unsubstituted heterocycloalkyl containing at least one O atom in the ring that is substituted or unsubstituted tetrahydrofuranyl, substituted or unsubstituted dihydrofuranyl, substituted or unsubstituted oxazolidinonyl, substituted or unsubstituted tetrahydropyranyl, substituted or unsubstituted dihydropyranyl, substituted or unsubstituted tetrahydrothiopyranyl, substituted or unsubstituted morpholinyl, substituted or unsubstituted oxetanyl, substituted or unsubstituted oxepanyl, substituted or unsubstituted oxazepinyl, or substituted or unsubstituted dioxanyl.

In some embodiments, R^{a} is a substituted or unsubstituted 5-membered heteroaryl that is substituted or unsubstituted furanyl, substituted or unsubstituted thienyl, substituted or unsubstituted pyrrolyl, substituted or unsubstituted oxazolyl, substituted or unsubstituted thiazolyl, substituted or unsubstituted imidazolyl, substituted or unsubstituted pyrazolyl, substituted or unsubstituted triazolyl, substituted or unsubstituted tetrazolyl, substituted or unsubstituted isoxazolyl, substituted or unsubstituted isothiazolyl, substituted or unsubstituted oxadiazolyl, or substituted or unsubstituted thiadiazolyl.

In some embodiments, the compound (e.g., a prodrug of Compound 1) has one of the following structures:

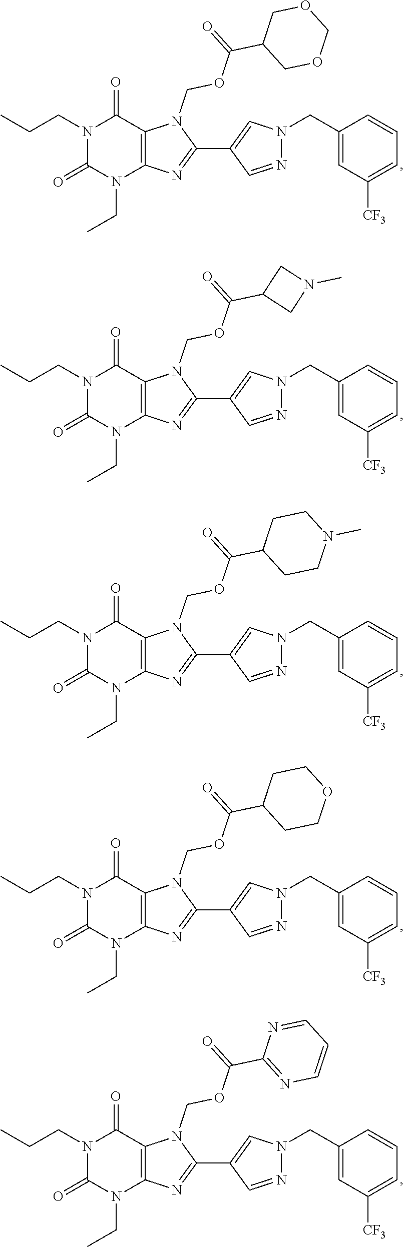 [SMILES: CCCn1c(=O)c2c(nc(-c3cnn(Cc4cccc(C(F)(F)F)c4)c3)n2COC(=O)C2COCOC2)n(CC)c1=O ; CCCn1c(=O)c2c(nc(-c3cnn(Cc4cccc(C(F)(F)F)c4)c3)n2COC(=O)C2CN(C)C2)n(CC)c1=O ; CCCn1c(=O)c2c(nc(-c3cnn(Cc4cccc(C(F)(F)F)c4)c3)n2COC(=O)C2CCN(C)CC2)n(CC)c1=O ; CCCn1c(=O)c2c(nc(-c3cnn(Cc4cccc(C(F)(F)F)c4)c3)n2COC(=O)C2CCOCC2)n(CC)c1=O ; CCCn1c(=O)c2c(nc(-c3cnn(Cc4cccc(C(F)(F)F)c4)c3)n2COC(=O)c2ncccn2)n(CC)c1=O]

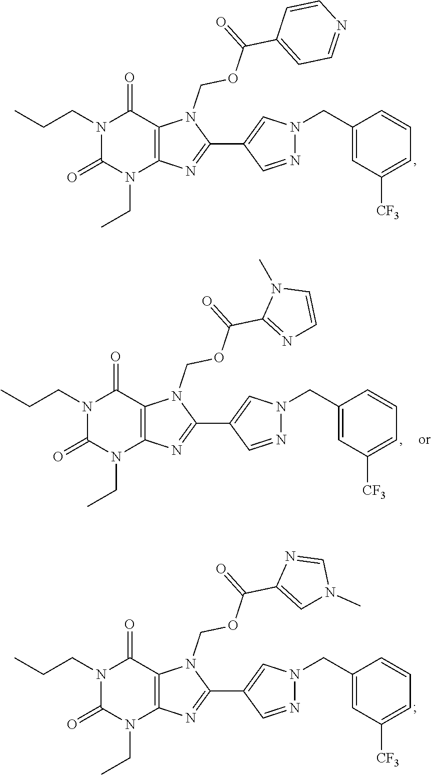 [SMILES: CCCn1c(=O)c2c(nc(-c3cnn(Cc4cccc(C(F)(F)F)c4)c3)n2COC(=O)c2ccncc2)n(CC)c1=O ; CCCn1c(=O)c2c(nc(-c3cnn(Cc4cccc(C(F)(F)F)c4)c3)n2COC(=O)c2nccn2C)n(CC)c1=O ; CCCn1c(=O)c2c(nc(-c3cnn(Cc4cccc(C(F)(F)F)c4)c3)n2COC(=O)c2cn(C)cn2)n(CC)c1=O]

or a pharmaceutically acceptable salt or solvate thereof.

In some embodiments, R¹ is ethyl; R² is n-propyl; R³ is 3-(trifluoromethyl)phenyl; and R⁵ is —C(═O)—(C(R¹⁰)₂O)ₘ—R¹¹, —C(═O)—(CH₂CH₂O)ₙ—R¹¹, or —C(═O)—OR⁷.

In some embodiments, the compound (e.g., a prodrug of Compound 1) has one of the following structures:

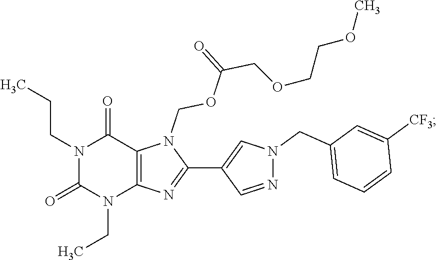 [SMILES: CCCn1c(=O)c2c(nc(-c3cnn(Cc4cccc(C(F)(F)F)c4)c3)n2COC(=O)COCCOC)n(CC)c1=O]

or a pharmaceutically acceptable salt or solvate thereof.

In some embodiments, R¹ is ethyl; R² is n-propyl; R³ is 3-(trifluoromethyl)phenyl; R⁵ is substituted or unsubstituted C₃-C₁₀cycloalkyl, substituted or unsubstituted C₂-C₁₀heterocycloalkyl, substituted or unsubstituted phenyl, substituted or unsubstituted heteroaryl, -alkyl-(substituted or unsubstituted phenyl), -alkyl-(substituted or unsubstituted heteroaryl), -alkyl-(substituted or unsubstituted cycloalkyl),-alkyl-(substituted or unsubstituted heterocycloalkyl). In some embodiments, R⁵ is —CH₂-(substituted or unsubstituted C₂-C₈heterocycloalkyl). In some embodiments, R⁵ is —CH₂-(substituted C₅-C₆heterocycloalkyl). In some embodiments, R⁵ is

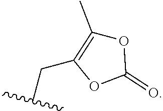 [SMILES: *Cc1oc(=O)oc1C]

In some embodiments, substituted means t at the referenced group is substituted with one or more additional groups individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, substituted means that the referenced group is substituted with one or more additional groups individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, and heterocycloalkyl. In yet other embodiments, substituted means that the referenced group is substituted with one or more additional groups individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, fluoroalkyl, alkoxy, and fluoroalkoxy.

In some embodiments, the compound (e.g., a prodrug of Compound 1) has the following structure:

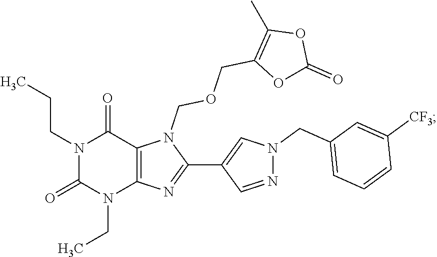 [SMILES: CCCn1c(=O)c2c(nc(-c3cnn(Cc4cccc(C(F)(F)F)c4)c3)n2COCc2oc(=O)oc2C)n(CC)c1=O]

or a pharmaceutically acceptable salt or solvate thereof.

In some embodiments, compounds of Formula (B) (e.g., prodrugs of Compound 1) include those presented in Table 2. In some embodiments, the compounds described herein can be prepared according to procedures described in WO 2019/173380, published Sep. 12, 2019, which procedures are incorporated herein by reference in their entirety.

TABLE 2

| Compound | Structure | Name |
|---|---|---|
| C | 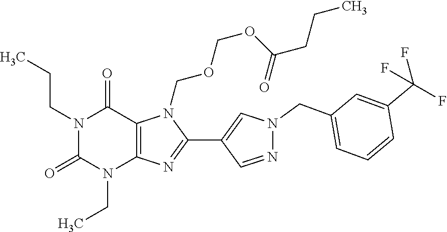 [SMILES: CCCC(=O)OCOCn1c(-c2cnn(Cc3cccc(C(F)(F)F)c3)c2)nc2c1c(=O)n(CCC)c(=O)n2CC] | ((3-ethyl-2,6-dioxo-1-propyl- 8-(1-(3- (trifluoromethyl)benzyl)-1H- pyrazol-4-yl)-1,2,3,6- tetrahydro-7H-purin-7- yl)methoxy)methyl butyrate |
| D | 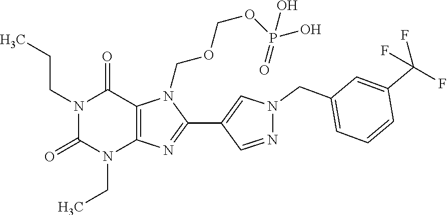 [SMILES: CCCn1c(=O)c2c(nc(-c3cnn(Cc4cccc(C(F)(F)F)c4)c3)n2COCOP(=O)(O)O)n(CC)c1=O] | ((3-ethyl-2,6-dioxo-1-propyl- 8-(1-(3- (trifluoromethyl)benzyl)-1H- pyrazol-4-yl)-1,2,3,6- tetrahydro-7H-purin-7- yl)methoxy)methyl dihydrogen phosphate |
| E | 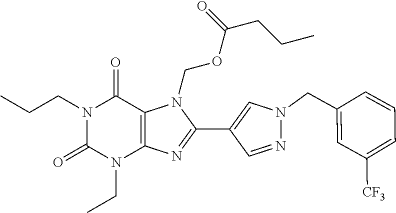 [SMILES: CCCC(=O)OCn1c(-c2cnn(Cc3cccc(C(F)(F)F)c3)c2)nc2c1c(=O)n(CCC)c(=O)n2CC] | (3-ethyl-2,6-dioxo-1-propyl- 8-(1-(3- (trifluoromethyl)benzyl)-1H- pyrazol-4-yl)-1,2,3,6- tetrahydro-7H-purin-7- yl)methyl butyrate |
| J | 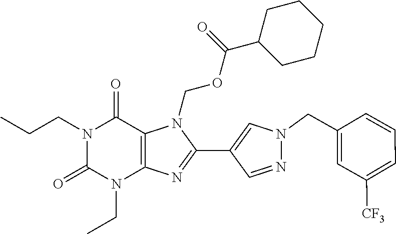 [SMILES: CCCn1c(=O)c2c(nc(-c3cnn(Cc4cccc(C(F)(F)F)c4)c3)n2COC(=O)C2CCCCC2)n(CC)c1=O] | (3-ethyl-2,6-dioxo-1-propyl- 8-(1-(3- (trifluoromethyl)benzyl)-1H- pyrazol-4-yl)-1,2,3,6- tetrahydro-7H-purin-7- yl)methyl cyclohexanecarboxylate |
| L | 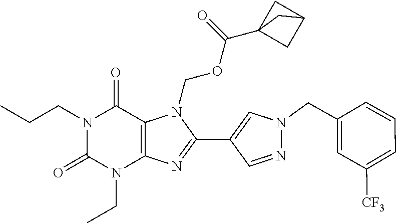 [SMILES: CCCn1c(=O)c2c(nc(-c3cnn(Cc4cccc(C(F)(F)F)c4)c3)n2COC(=O)C23CC(C2)C3)n(CC)c1=O] | (3-ethyl-2,6-dioxo-1-propyl- 8-(1-(3- (trifluoromethyl)benzyl)-1H- pyrazol-4-yl)-1,2,3,6- tetrahydro-7H-purin-7- yl)methyl bicyclo[1.1.1]pentane-1- carboxylate |
| N | 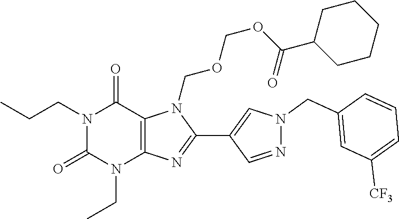 [SMILES: CCCn1c(=O)c2c(nc(-c3cnn(Cc4cccc(C(F)(F)F)c4)c3)n2COCOC(=O)C2CCCCC2)n(CC)c1=O] | ((3-ethyl-2,6-dioxo-1-propyl- 8-(1-(3- (trifluoromethyl)benzyl)-1H- pyrazol-4-yl)-1,2,3,6- tetrahydro-7H-purin-7- yl)methoxy)methyl cyclohexanecarboxylate |
| R | 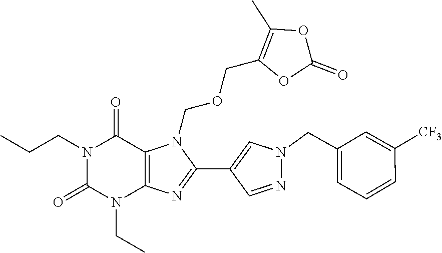 | 3-ethyl-7-(((5-methyl-2-oxo- 1,3-dioxol-4- yl)methoxy)methyl)-1- propyl-8-(1-(3- (trifluoromethyl)benzyl)-1H- pyrazol-4-yl)-3,7-dihydro- 1H-purine-2,6-dione |
| U | 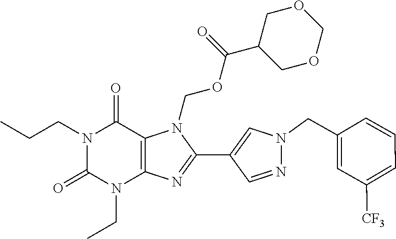 [SMILES: CCCn1c(=O)c2c(nc(-c3cnn(Cc4cccc(C(F)(F)F)c4)c3)n2COC(=O)C2COCOC2)n(CC)c1=O] | (3-ethyl-2,6-dioxo-1-propyl- 8-(1-(3- (trifluoromethyl)benzyl)-1H- pyrazol-4-yl)-1,2,3,6- tetrahydro-7H-purin-7- yl)methyl 1,3-dioxane-5- carboxylate |
| V | 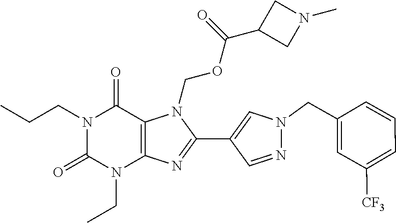 [SMILES: CCCn1c(=O)c2c(nc(-c3cnn(Cc4cccc(C(F)(F)F)c4)c3)n2COC(=O)C2CN(C)C2)n(CC)c1=O] | (3-ethyl-2,6-dioxo-1-propyl- 8-(1-(3- (trifluoromethyl)benzyl)-1H- pyrazol-4-yl)-1,2,3,6- tetrahydro-7H-purin-7- yl)methyl 1-methylazetidine- 3-carboxylate |
| AA | 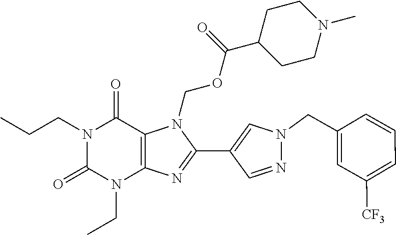 [SMILES: CCCn1c(=O)c2c(nc(-c3cnn(Cc4cccc(C(F)(F)F)c4)c3)n2COC(=O)C2CCN(C)CC2)n(CC)c1=O] | (3-ethyl-2,6-dioxo-1-propyl- 8-(1-(3- (trifluoromethyl)benzyl)-1H- pyrazol-4-yl)-1,2,3,6- tetrahydro-7H-purin-7- yl)methyl 1-methylpiperidine- 4-carboxylate |
| BB | 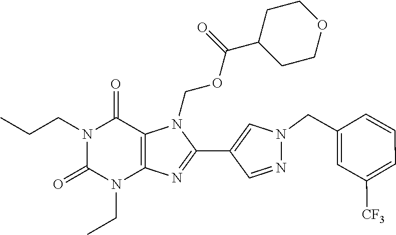 [SMILES: CCCn1c(=O)c2c(nc(-c3cnn(Cc4cccc(C(F)(F)F)c4)c3)n2COC(=O)C2CCOCC2)n(CC)c1=O] | (3-ethyl-2,6-dioxo-1-propyl- 8-(1-(3- (trifluoromethyl)benzyl)-1H- pyrazol-4-yl)-1,2,3,6- tetrahydro-7H-purin-7- yl)methyl tetrahydro-2H- pyran-4-carboxylate |
| CC | 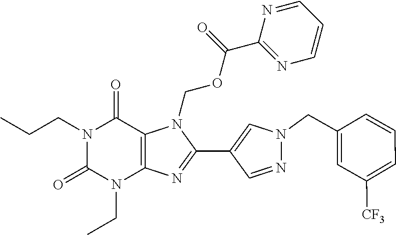 [SMILES: CCCn1c(=O)c2c(nc(-c3cnn(Cc4cccc(C(F)(F)F)c4)c3)n2COC(=O)c2ncccn2)n(CC)c1=O] | (3-ethyl-2,6-dioxo-1-propyl- 8-(1-(3- (trifluoromethyl)benzyl)-1H- pyrazol-4-yl)-1,2,3,6- tetrahydro-7H-purin-7- yl)methyl pyrimidine-2- carboxylate |
| DD | 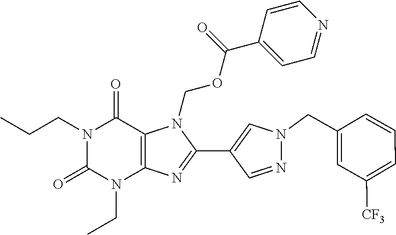 [SMILES: CCCn1c(=O)c2c(nc(-c3cnn(Cc4cccc(C(F)(F)F)c4)c3)n2COC(=O)c2ccncc2)n(CC)c1=O] | (3-ethyl-2,6-dioxo-1-propyl- 8-(1-(3- (trifluoromethyl)benzyl)-1H- pyrazol-4-yl)-1,2,3,6- tetrahydro-7H-purin-7- yl)methyl isonicotinate |
| EE | 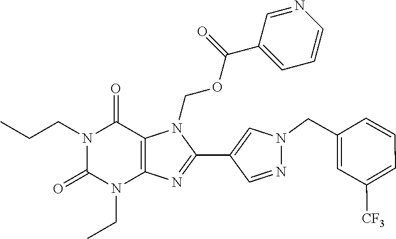 [SMILES: CCCn1c(=O)c2c(nc(-c3cnn(Cc4cccc(C(F)(F)F)c4)c3)n2COC(=O)c2cccnc2)n(CC)c1=O] | (3-ethyl-2,6-dioxo-1-propyl- 8-(1-(3- (trifluoromethyl)benzyl)-1H- pyrazol-4-yl)-1,2,3,6- tetrahydro-7H-purin-7- yl)methyl nicotinate |
| FF | 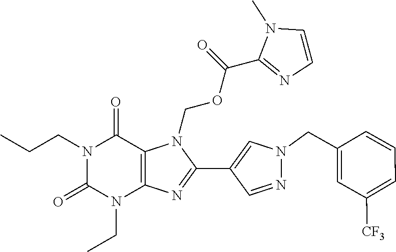 [SMILES: CCCn1c(=O)c2c(nc(-c3cnn(Cc4cccc(C(F)(F)F)c4)c3)n2COC(=O)c2nccn2C)n(CC)c1=O] | (3-ethyl-2,6-dioxo-1-propyl- 8-(1-(3- (trifluoromethyl)benzyl)-1H- pyrazol-4-yl)-1,2,3,6- tetrahydro-7H-purin-7- yl)methyl 1-methyl-1H- imidazole-2-carboxylate |
| GG | 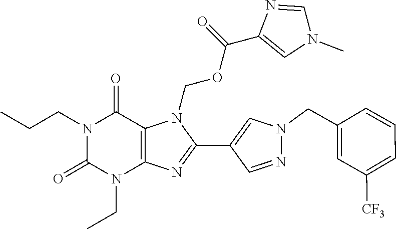 [SMILES: CCCn1c(=O)c2c(nc(-c3cnn(Cc4cccc(C(F)(F)F)c4)c3)n2COC(=O)c2cn(C)cn2)n(CC)c1=O] | (3-ethyl-2,6-dioxo-1-propyl- 8-(1-(3- (trifluoromethyl)benzyl)-1H- pyrazol-4-yl)-1,2,3,6- tetrahydro-7H-purin-7- yl)methyl 1-methyl-1H- imidazole-4-carboxylate |

In one aspect, the prodrug of Compound 1 has any one of the following structures:

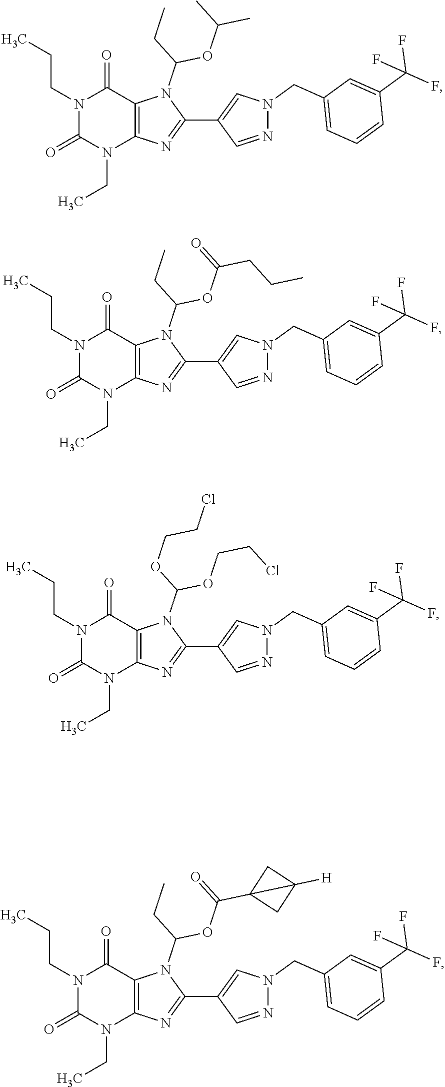 [SMILES: CCCn1c(=O)c2c(nc(-c3cnn(Cc4cccc(C(F)(F)F)c4)c3)n2C(CC)OC(C)C)n(CC)c1=O ; CCCC(=O)OC(CC)n1c(-c2cnn(Cc3cccc(C(F)(F)F)c3)c2)nc2c1c(=O)n(CCC)c(=O)n2CC ; CCCn1c(=O)c2c(nc(-c3cnn(Cc4cccc(C(F)(F)F)c4)c3)n2C(OCCCl)OCCCl)n(CC)c1=O ; CCCn1c(=O)c2c(nc(-c3cnn(Cc4cccc(C(F)(F)F)c4)c3)n2C(CC)OC(=O)C23CC2C3)n(CC)c1=O]

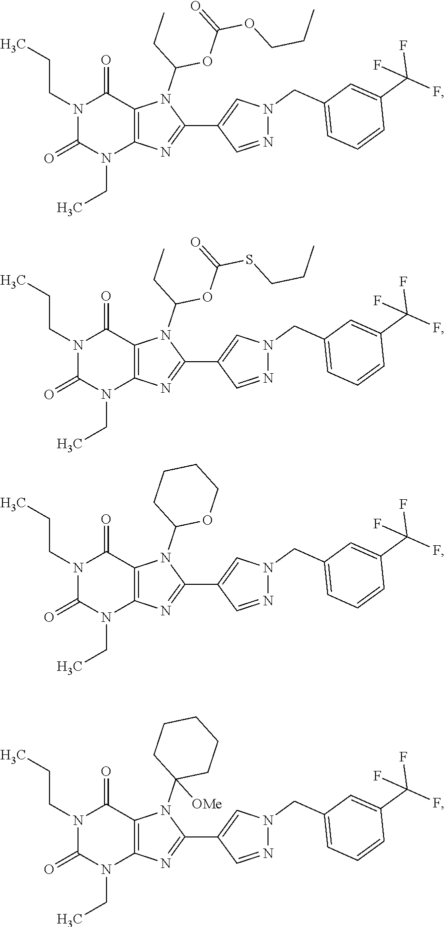 [SMILES: CCCOC(=O)OC(CC)n1c(-c2cnn(Cc3cccc(C(F)(F)F)c3)c2)nc2c1c(=O)n(CCC)c(=O)n2CC ; CCCSC(=O)OC(CC)n1c(-c2cnn(Cc3cccc(C(F)(F)F)c3)c2)nc2c1c(=O)n(CCC)c(=O)n2CC ; CCCn1c(=O)c2c(nc(-c3cnn(Cc4cccc(C(F)(F)F)c4)c3)n2C2CCCCO2)n(CC)c1=O ; CCCn1c(=O)c2c(nc(-c3cnn(Cc4cccc(C(F)(F)F)c4)c3)n2C2(OC)CCCCC2)n(CC)c1=O]

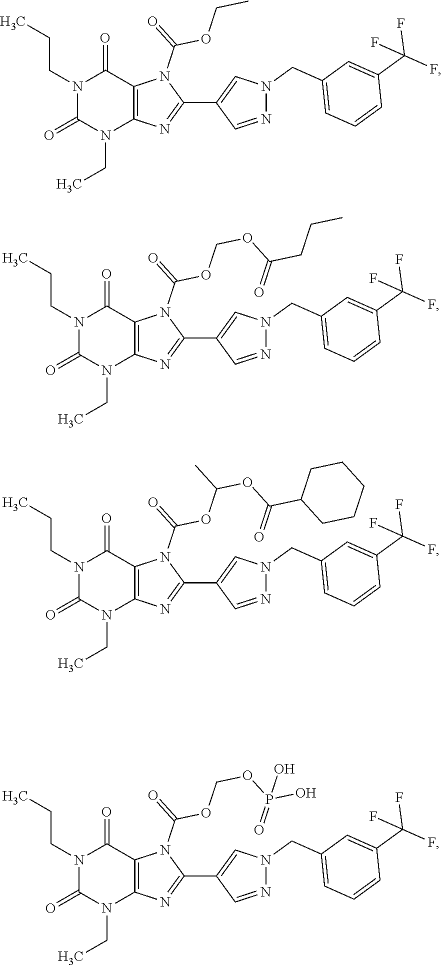 [SMILES: CCCn1c(=O)c2c(nc(-c3cnn(Cc4cccc(C(F)(F)F)c4)c3)n2C(=O)OCC)n(CC)c1=O ; CCCC(=O)OCOC(=O)n1c(-c2cnn(Cc3cccc(C(F)(F)F)c3)c2)nc2c1c(=O)n(CCC)c(=O)n2CC ; CCCn1c(=O)c2c(nc(-c3cnn(Cc4cccc(C(F)(F)F)c4)c3)n2C(=O)OC(C)OC(=O)C2CCCCC2)n(CC)c1=O ; CCCn1c(=O)c2c(nc(-c3cnn(Cc4cccc(C(F)(F)F)c4)c3)n2C(=O)OCOP(=O)(O)O)n(CC)c1=O]

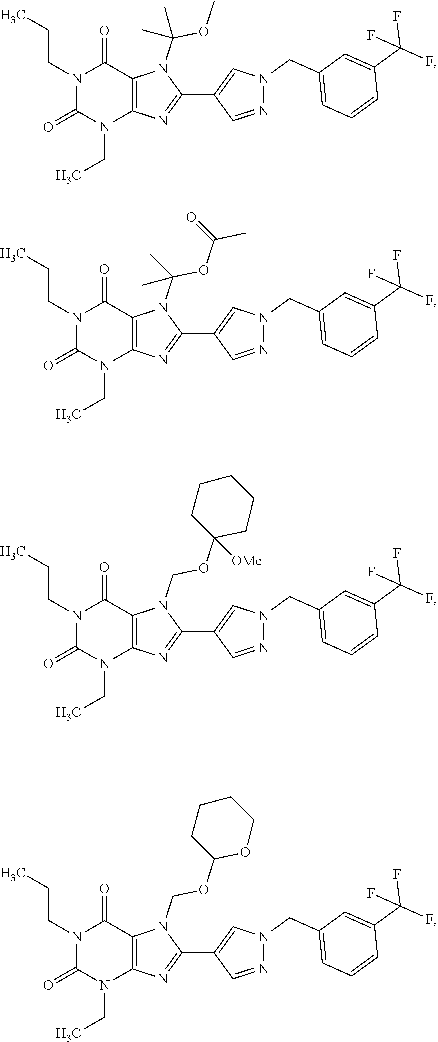 [SMILES: CCCn1c(=O)c2c(nc(-c3cnn(Cc4cccc(C(F)(F)F)c4)c3)n2C(C)(C)OC)n(CC)c1=O ; CCCn1c(=O)c2c(nc(-c3cnn(Cc4cccc(C(F)(F)F)c4)c3)n2C(C)(C)OC(C)=O)n(CC)c1=O ; CCCn1c(=O)c2c(nc(-c3cnn(Cc4cccc(C(F)(F)F)c4)c3)n2COC2(OC)CCCCC2)n(CC)c1=O ; CCCn1c(=O)c2c(nc(-c3cnn(Cc4cccc(C(F)(F)F)c4)c3)n2COC2CCCCO2)n(CC)c1=O]

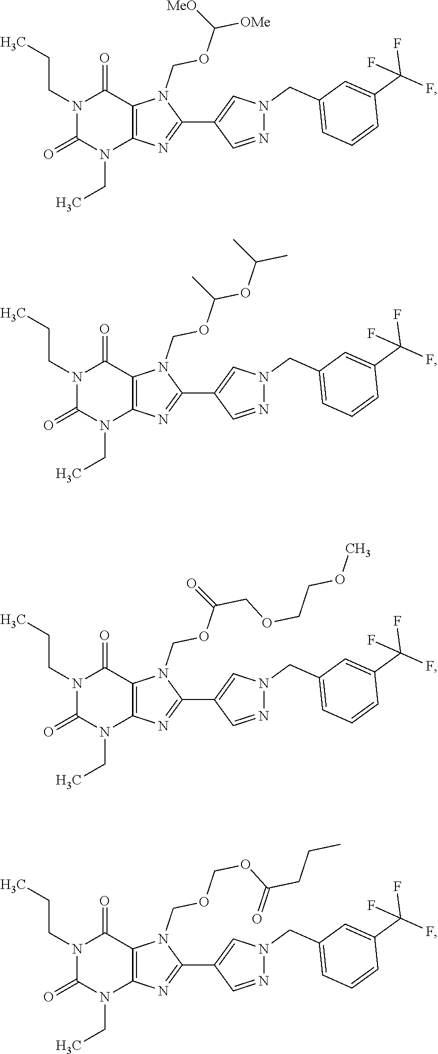 [SMILES: CCCn1c(=O)c2c(nc(-c3cnn(Cc4cccc(C(F)(F)F)c4)c3)n2COC(OC)OC)n(CC)c1=O ; CCCn1c(=O)c2c(nc(-c3cnn(Cc4cccc(C(F)(F)F)c4)c3)n2COC(C)OC(C)C)n(CC)c1=O ; CCCn1c(=O)c2c(nc(-c3cnn(Cc4cccc(C(F)(F)F)c4)c3)n2COC(=O)COCCOC)n(CC)c1=O ; CCCC(=O)OCOCn1c(-c2cnn(Cc3cccc(C(F)(F)F)c3)c2)nc2c1c(=O)n(CCC)c(=O)n2CC]

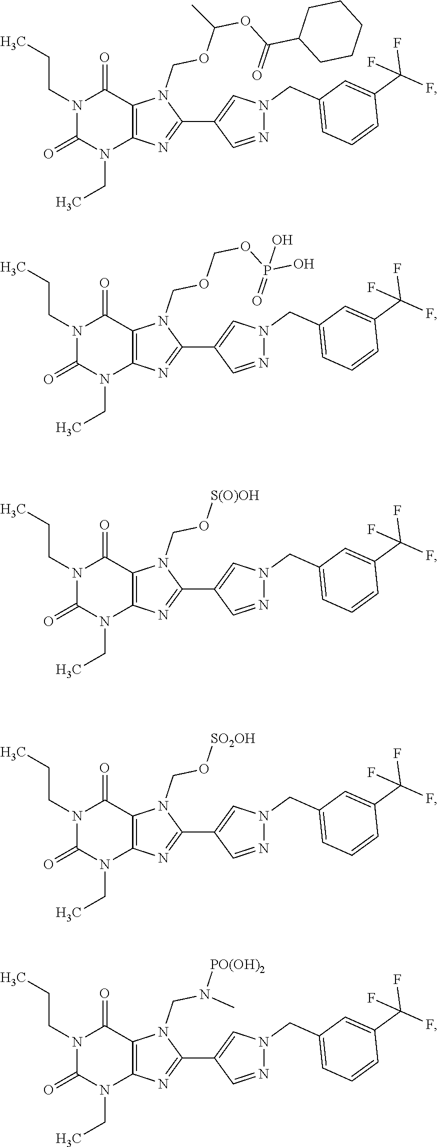

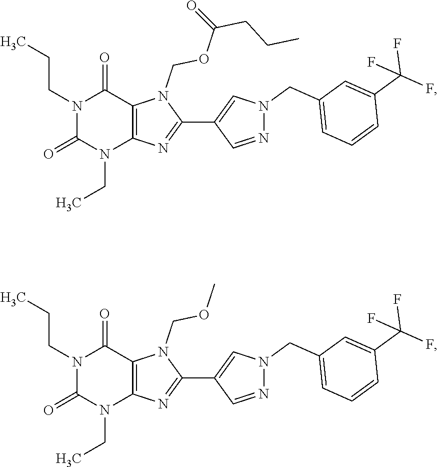 [SMILES: CCCC(=O)OCn1c(-c2cnn(Cc3cccc(C(F)(F)F)c3)c2)nc2c1c(=O)n(CCC)c(=O)n2CC ; CCCn1c(=O)c2c(nc(-c3cnn(Cc4cccc(C(F)(F)F)c4)c3)n2COC)n(CC)c1=O]

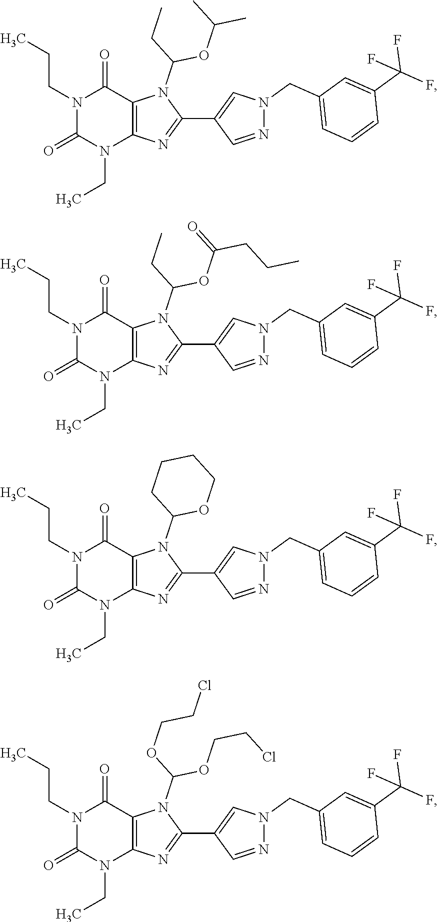 [SMILES: CCCn1c(=O)c2c(nc(-c3cnn(Cc4cccc(C(F)(F)F)c4)c3)n2C(CC)OC(C)C)n(CC)c1=O ; CCCC(=O)OC(CC)n1c(-c2cnn(Cc3cccc(C(F)(F)F)c3)c2)nc2c1c(=O)n(CCC)c(=O)n2CC ; CCCn1c(=O)c2c(nc(-c3cnn(Cc4cccc(C(F)(F)F)c4)c3)n2C2CCCCO2)n(CC)c1=O ; CCCn1c(=O)c2c(nc(-c3cnn(Cc4cccc(C(F)(F)F)c4)c3)n2C(OCCCl)OCCCl)n(CC)c1=O]

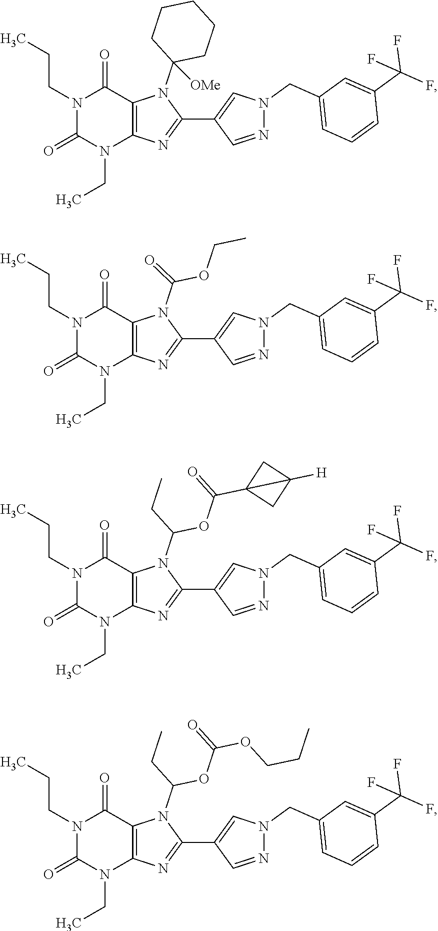 [SMILES: CCCn1c(=O)c2c(nc(-c3cnn(Cc4cccc(C(F)(F)F)c4)c3)n2C2(OC)CCCCC2)n(CC)c1=O ; CCCn1c(=O)c2c(nc(-c3cnn(Cc4cccc(C(F)(F)F)c4)c3)n2C(=O)OCC)n(CC)c1=O ; CCCn1c(=O)c2c(nc(-c3cnn(Cc4cccc(C(F)(F)F)c4)c3)n2C(CC)OC(=O)C23CC2C3)n(CC)c1=O ; CCCOC(=O)OC(CC)n1c(-c2cnn(Cc3cccc(C(F)(F)F)c3)c2)nc2c1c(=O)n(CCC)c(=O)n2CC]

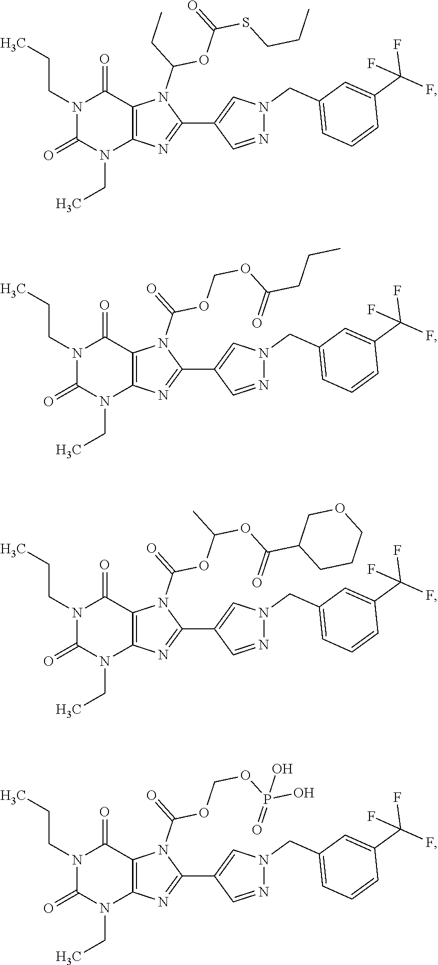 [SMILES: CCCSC(=O)OC(CC)n1c(-c2cnn(Cc3cccc(C(F)(F)F)c3)c2)nc2c1c(=O)n(CCC)c(=O)n2CC ; CCCC(=O)OCOC(=O)n1c(-c2cnn(Cc3cccc(C(F)(F)F)c3)c2)nc2c1c(=O)n(CCC)c(=O)n2CC ; CCCn1c(=O)c2c(nc(-c3cnn(Cc4cccc(C(F)(F)F)c4)c3)n2C(=O)OC(C)OC(=O)C2CCCOC2)n(CC)c1=O ; CCCn1c(=O)c2c(nc(-c3cnn(Cc4cccc(C(F)(F)F)c4)c3)n2C(=O)OCOP(=O)(O)O)n(CC)c1=O]

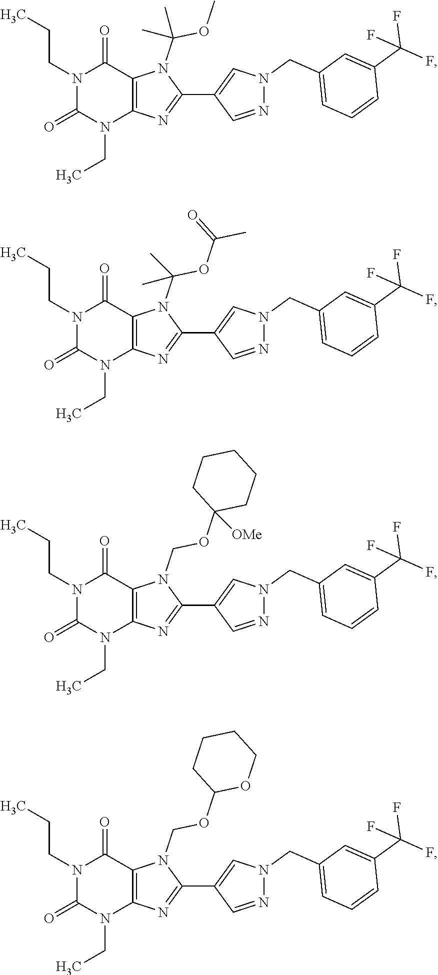 [SMILES: CCCn1c(=O)c2c(nc(-c3cnn(Cc4cccc(C(F)(F)F)c4)c3)n2C(C)(C)OC)n(CC)c1=O ; CCCn1c(=O)c2c(nc(-c3cnn(Cc4cccc(C(F)(F)F)c4)c3)n2C(C)(C)OC(C)=O)n(CC)c1=O ; CCCn1c(=O)c2c(nc(-c3cnn(Cc4cccc(C(F)(F)F)c4)c3)n2COC2(OC)CCCCC2)n(CC)c1=O ; CCCn1c(=O)c2c(nc(-c3cnn(Cc4cccc(C(F)(F)F)c4)c3)n2COC2CCCCO2)n(CC)c1=O]

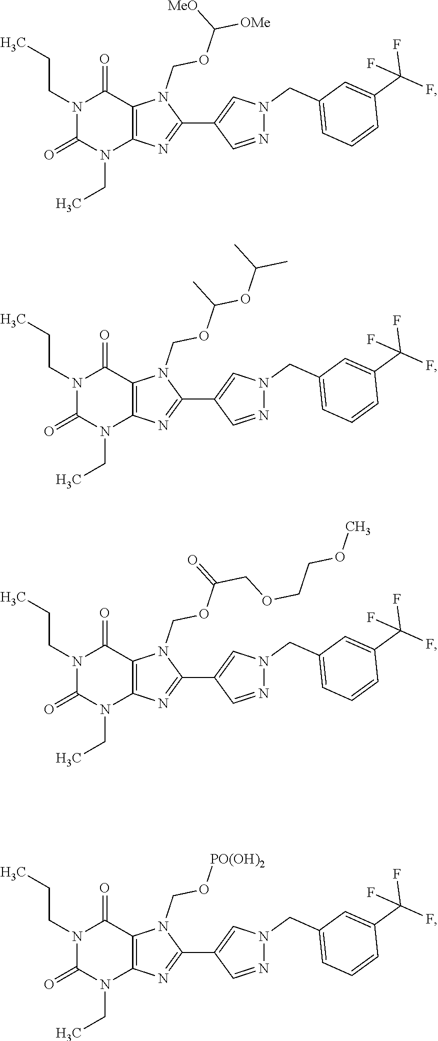

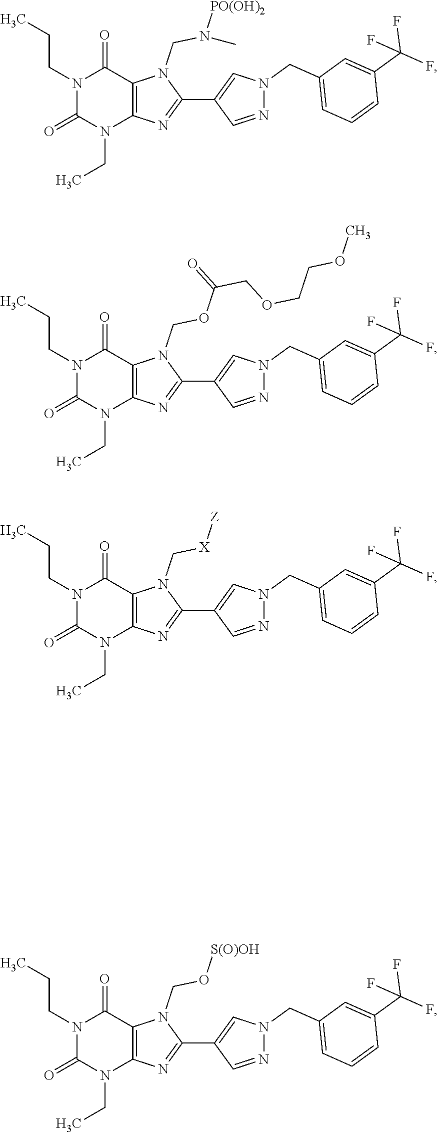

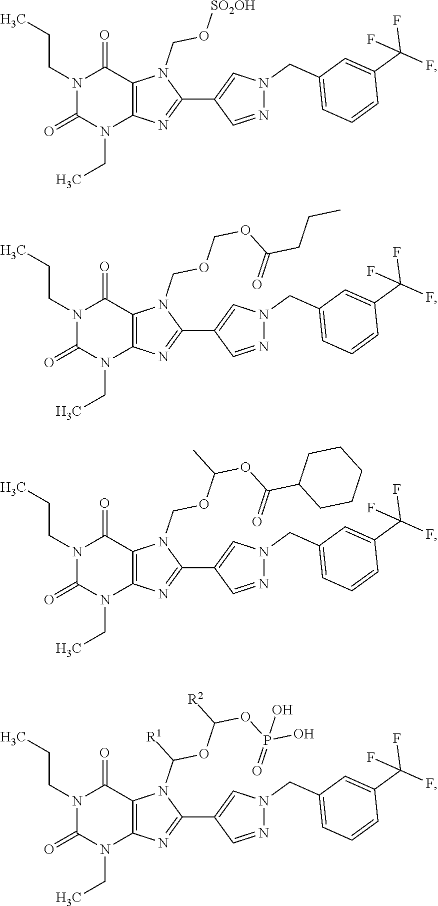

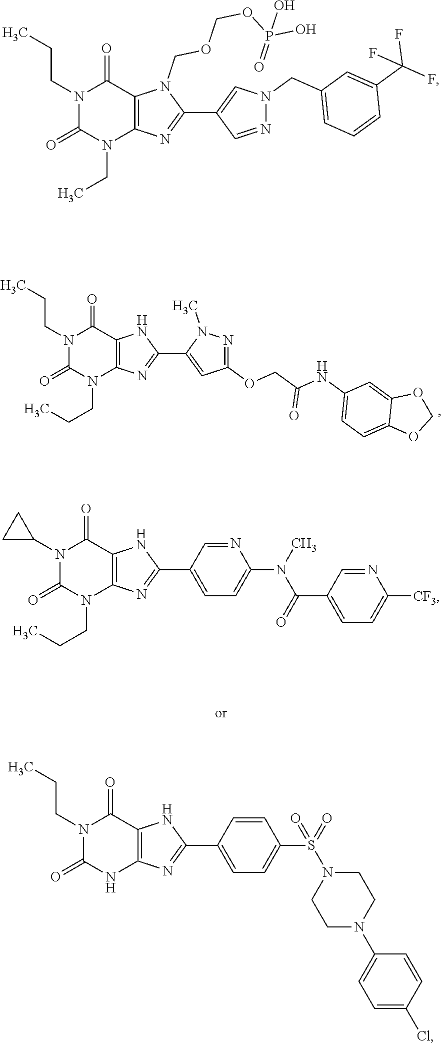 [SMILES: CCCn1c(=O)c2c(nc(-c3cnn(Cc4cccc(C(F)(F)F)c4)c3)n2COCOP(=O)(O)O)n(CC)c1=O ; CCCn1c(=O)c2[nH]c(-c3cc(OCC(=O)Nc4ccc5c(c4)OCO5)nn3C)nc2n(CCC)c1=O ; CCCn1c(=O)n(C2CC2)c(=O)c2[nH]c(-c3ccc(N(C)C(=O)c4ccc(C(F)(F)F)nc4)nc3)nc21 ; CCCn1c(=O)[nH]c2nc(-c3ccc(S(=O)(=O)N4CCN(c5ccc(Cl)cc5)CC4)cc3)[nH]c2c1=O]

or any pharmaceutically acceptable salt or solvate thereof.

In some embodiments, a prodrug of Compound 1 has the following structure

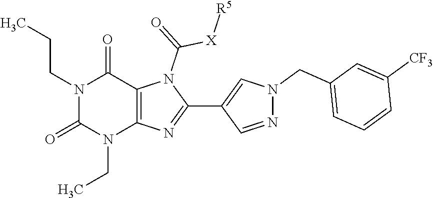

or a pharmaceutically acceptable salt or solvate thereof, wherein:
X is O, S, NHR′, NR′R″, CH₂, CHR′, or CR′R″;
R′ is C₁-C₆alkyl;
R″ is C₁-C₆alkyl;
R⁵ is hydrogen, R⁷, —C(═O)R⁷, —C(═O)—OR⁷, —C(═O)N(R⁷)(R⁸), —C(═O)—SR⁷, or —P(═O)(OR⁹)₂;
R⁷ is substituted or unsubstituted alkyl, substituted or unsubstituted heteroalkyl, substituted or unsubstituted C₃-C₁₀cycloalkyl, substituted or unsubstituted C₂-C₁₀heterocycloalkyl, substituted or unsubstituted phenyl, substituted or unsubstituted heteroaryl, -alkyl-(substituted or unsubstituted phenyl), -alkyl-(substituted or unsubstituted heteroaryl), -alkyl-(substituted or unsubstituted cycloalkyl),-alkyl-(substituted or unsubstituted heterocycloalkyl), —(C(R¹⁰)₂O)ₘ—R¹¹, —(CH₂CH₂O)ₙ—R¹¹, or —(C(R¹⁰)₂)ₚ—OR¹¹;
R⁸ is hydrogen or alkyl;
or R⁷ and R⁸ are taken together with the nitrogen atom to which they are attached to form a substituted or unsubstituted C₂-C₁₀heterocycloalkyl;
each R⁹ is independently selected from hydrogen and alkyl;
each R¹⁰ is independently selected from hydrogen and alkyl;
R¹¹ is hydrogen, substituted or unsubstituted alkyl, substituted or unsubstituted heteroalkyl, substituted or unsubstituted C₂-C₁₀heterocycloalkyl, —C(═O)R¹², —C(═O)—OR¹², —C(═O)N(R¹²)(RI), —C(═O)—SR¹², or —P(═O)(OR⁹)₂;
R¹² is hydrogen, substituted or unsubstituted alkyl, substituted or unsubstituted heteroalkyl, substituted or unsubstituted C₃-C₁₀cycloalkyl, substituted or unsubstituted C₂-C₁₀heterocycloalkyl, substituted or unsubstituted phenyl, substituted or unsubstituted heteroaryl, -alkyl-(substituted or unsubstituted phenyl), or -alkyl-(substituted or unsubstituted heteroaryl);
m is 1, 2, 3, 4, 5, or 6;
n is 1, 2, 3, 4, 5, or 6;
p is 1, 2, 3, 4, 5, or 6;
wherein substituted means that the referenced group is substituted with one or more additional groups individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone.

Prodrugs of these A_{2B} adenosine receptor antagonists can be designed and synthesized in a similar way to the prodrugs of the Compound 1 by substituting the xanthine at the 7-position.

Additional compounds for use in the combination therapies described herein include any one of the compounds described in WO 2019/135259. Such compounds include: 5-propyl-2-[1-[[3-(trifluoromethyl)phenyl]methyl]pyrazol-4-yl]-3H-imidazo[2,1-b]purin-4-one (A1), 2-(1-benzylpyrazol-4-yl)-5-propyl-3H-imidazo[2,1-b]purin-4-one (A2), 5-methyl-2-[1-[[3-(trifluoromethyl)phenyl]methyl]pyrazol-4-yl]-3H-imidazo[2,1-b]purin-4-one (A3), 5-propyl-2-[1-[2-[3-(trifluoromethyl)phenyl]ethyl]pyrazol-4-yl]-3H-imidazo[2,1-b]purin-4-one (A4), 2-[1-[2-(3-fluorophenyl)ethyl]pyrazol-4-yl]-5-propyl-3H-imidazo[2,1-b]purin-4-one (A5), 2-(1-methylpyrazol-4-yl)-5-propyl-3H-imidazo[2,1-b] purin-4-one (A6), 2-[1-(1,1-dimethylpropyl)pyrazol-4-yl]-5-propyl-3H-imidazo[2,1-b]purin-4-one (A7), N-isopropyl-3-[3-[4-(4-oxo-5-propyl-3H-imidazo[2,1-b]purin-2-yl)pyrazol-1-yl]prop-1-ynyl]benzamide (B1), Ethyl 3-[3-[4-(4-oxo-5-propyl-3H-imidazo[2,1-b]purin-2-yl)pyrazol-1-yl]prop-1-ynyl]benzoate (B2), 5-propyl-2-[1-[3-[3-(trifluoromethoxy)phenyl]prop-2-ynyl]pyrazol-4-yl]-3H-imidazo[2,1-b]purin-4-one (B3), Ethyl 4-methyl-3-[3-[4-(4-oxo-5-propyl-3H-imidazo[2,1-b]purin-2-yl)pyrazol-1-yl]prop-1-ynyl]benzoate (B6), 3-[[4-(4-oxo-5-propyl-3H-imidazo[2,1-b]purin-2-yl)pyrazol-1-yl]methyl] benzonitrile (B7), 2-(1-isopropylpyrazol-4-yl)-5-propyl-3H-imidazo[2,1-b]purin-4-one (B8), 2-(1-butylpyrazol-4-yl)-5-propyl-3H-imidazo[2,1-b]purin-4-one (B9), 2-(1-ethylpyrazol-4-yl)-5-propyl-3H-imidazo[2,1-b]purin-4-one (B10), 2-[1-(2-methoxyethyl)pyrazol-4-yl]-5-propyl-3H-imidazo[2,1-b]purin-4-one (B11), 2-[1-(2-dimethylaminoethyl)pyrazol-4-yl]-5-propyl-3H-imidazo[2,1-b]purin-4-one (B12), 5-propyl-2-(1-propylpyrazol-4-yl)-3H-imidazo[2,1-b]purin-4-one (B13), N,N-dimethyl-2-[4-(4-oxo-5-propyl-3H-imidazo[2,1-b]purin-2-yl)pyrazol-1-yl]acetamide (B14), 2-[1-(2-morpholinoethyl)pyrazol-4-yl]-5-propyl-3H-imidazo[2,1-b]purin-4-one (B15), 2-[1-(cyclobutylmethyl)pyrazol-4-yl]-5-propyl-3H-imidazo[2,1-b]purin-4-one (B16), 2-(1-isobutylpyrazol-4-yl)-5-propyl-3H-imidazo[2,1-b]purin-4-one (B17), 2-[1-(cyclopropylmethyl)pyrazol-4-yl]-5-propyl-3H-imidazo[2,1-b]purin-4-one (B18), 2-[1-(2,2-dimethylpropyl)pyrazol-4-yl]-5-propyl-3H-imidazo[2,1-b]purin-4-one(B19), 5-propyl-2-(1-sec-butylpyrazol-4-yl)-3H-imidazo[2,1-b]purin-4-one (B20), 5-propyl-2-[1-(tetrahydrofuran-2-ylmethyl)pyrazol-4-yl]-3H-imidazo[2,1-b]purin-4-one (B21), 2-[1-[[5-oxo-1-[2-(trifluoromethyl)-4-pyridyl]pyrrolidin-3-yl] methyl]pyrazol-4-yl]-5-propyl-3H-imidazo[2,1-b]purin-4-one (C1), 2-[1-[[5-oxo-1-[5-(trifluoromethyl)-3-pyridyl]pyrrolidin-3-yl]methyl]pyrazol-4-yl]-5-propyl-3H-imidazo[2,1-b]purin-4-one (C2), 2-[1-[[5-oxo-1-[3-(trifluoromethyl)phenyl]pyrrolidin-3-yl]methyl]pyrazol-4-yl]-5-propyl-3H-imidazo[2,1-b]purin-4-one (C3), 2-[1-[2-(1-piperidyl)ethyl]pyrazol-4-yl]-5-propyl-3H-imidazo[2,1-b]purin-4-one (D1), 2-[1-[[3-(hydroxymethyl)phenyl]methyl]pyrazol-4-yl]-5-propyl-3H-imidazo[2,1-b]purin-4-one (E1), 2-[1-[[3-(1-hydroxy-1-methyl-ethyl)phenyl]methyl]pyrazol-4-yl]-5-propyl-3H-imidazo[2,1-b]purin-4-one (F1), 3-[[4-(4-oxo-5-propyl-3H-imidazo[2,1-b]purin-2-yl)pyrazol-1-yl]methyl]benzoic acid (G1), 2-[4-(4-oxo-5-propyl-3H-imidazo[2,1-b]purin-2-yl)pyrazol-1-yl]acetic acid (H1), 2-[1-(2-hydroxy-2-methyl-propyl)pyrazol-4-yl]-5-propyl-3H-imidazo[2,1-b]purin-4-one (I1), 2-[1-(2,3-dihydroxypropyl)pyrazol-4-yl]-5-propyl-3H-imidazo[2,1-b]purin-4-one (J1), 2-(3,4-dimethoxyphenyl)-5-propyl-3H-imidazo[2,1-b]purin-4-one (K1), 5-propyl-2-[3-(trifluoromethyl)phenyl]-3H-imidazo[2,1-b]purin-4-one (K2), 2-[1-[(3-fluorophenyl)methyl]pyrazol-4-yl]-5-propyl-3H-imidazo[2,1-b]purin-4-one (K3), 2-[1-[(3-methoxyphenyl)methyl]pyrazol-4-yl]-5-propyl-3H-imidazo[2,1-b]purin-4-one (K4), 2-[4-[2-(1-piperidyl)ethoxy]phenyl]-5-propyl-3H-imidazo[2,1-b]purin-4-one (K5), 2-(5-methoxy-2-pyridyl)-5-propyl-3H-imidazo[2,1-b]purin-4-one (K6), 2-(4-ethoxyphenyl)-5-propyl-3H-imidazo[2,1-b]purin-4-one (K7), N-isopropyl-2-[4-(4-oxo-5-propyl-3H-imidazo[2,1-b]purin-2-yl)pyrazol-1-yl]acetamide (LI), N-(oxetan-3-yl)-2-[4-(4-oxo-5-propyl-3H-imidazo[2,1-b]purin-2-yl)pyrazol-1-yl]acetamide (L2), 4-propyl-2-[1-[[3-(trifluoromethyl)phenyl] methyl]pyrazol-4-yl]-1H-imidazo[2,1-f]purin-5-one (II), 2-[1-(2-furylmethyl)pyrazol-4-yl]-5-propyl-3H-imidazo[2,1-b]purin-4-one (III), 5-propyl-2-[1-(2-thienylmethyl)pyrazol-4-yl]-3H-imidazo[2,1-b]purin-4-one (IV), 2-[1-(oxazol-2-ylmethyl)pyrazol-4-yl]-5-propyl-3H-imidazo[2,1-b]purin-4-one (V), 2-[1-(isoxazol-5-ylmethyl)pyrazol-4-yl]-5-propyl-3H-imidazo[2,1-b]purin-4-one (VI), 2-[1-[(5-methyl-2-thienyl)methyl]pyrazol-4-yl]-5-propyl-3H-imidazo[2,1-b]purin-4-one (VII), 2-[1-[(3,5-difluorophenyl)methyl]pyrazol-4-yl]-5-propyl-3H-imidazo[2,1-b]purin-4-one (VIII), 5-propyl-2-[1-(4-pyridylmethyl)pyrazol-4-yl]-3H-imidazo[2,1-b]purin-4-one (IX), 5-propyl-2-[1-(3-pyridylmethyl)pyrazol-4-yl]-3H-imidazo[2,1-b]purin-4-one (X), 5-propyl-2-[1-(2-pyridylmethyl)pyrazol-4-yl]-3H-imidazo[2,1-b]purin-4-one (XI), 5-propyl-2-[1-(pyrimidin-5-ylmethyl)pyrazol-4-yl]-3H-imidazo[2,1-b]purin-4-one (XII), 5-propyl-2-[1-(pyridazin-4-ylmethyl)pyrazol-4-yl]-3H-imidazo[2,1-b]purin-4-one (XIII), 2-[1-[(1-oxoisoindolin-5-yl)methyl]pyrazol-4-yl]-5-propyl-3H-imidazo[2,1-b]purin-4-one (XIV), 2-[1-[(2-methyl-1-oxo-isoindolin-5-yl)methyl]pyrazol-4-yl]-5-propyl-3H-imidazo[2,1-b]purin-4-one (XV), 2-[1-[(1-oxo-3,4-dihydro-2H-isoquinolin-6-yl)methyl]pyrazol-4-yl]-5-propyl-3H-imidazo[2,1-b]purin-4-one (XVI), 2-[1-[(2-oxo-3,4-dihydro-1H-quinolin-6-yl)methyl]pyrazol-4-yl]-5-propyl-3H-imidazo[2,1-b]purin-4-one (XVII), 5-propyl-2-[1-(quinoxalin-6-ylmethyl)pyrazol-4-yl]-3H-imidazo[2,1-b]purin-4-one (XVIII), 2-[1-(2-naphthylmethyl)pyrazol-4-yl]-5-propyl-3H-imidazo[2,1-b]purin-4-one (XIX), 2-[1-[[3-(azetidin-3-yl)phenyl]methyl]pyrazol-4-yl]-5-propyl-3H-imidazo[2,1-b]purin-4-one (XX), 2-[1-[[3-(2-methoxyethoxy)phenyl]methyl]pyrazol-4-yl]-5-propyl-3H-imidazo[2,1-b]purin-4-one (XXI), 2-[1-[[4-(2-methoxyethoxy)phenyl]methyl]pyrazol-4-yl]-5-propyl-3H-imidazo[2,1-b]purin-4-one (XXII), 5-propyl-2-[1-[3-[3-(trifluoromethyl)phenyl]prop-2-ynyl]pyrazol-4-yl]-3H-imidazo[2,1-b]purin-4-one (XXIII), 2-[1-[3-(3-fluorophenyl)prop-2-ynyl]pyrazol-4-yl]-5-propyl-3H-imidazo[2,1-b]purin-4-one (XXIV), 2-[1-[3-(4-fluorophenyl)prop-2-ynyl]pyrazol-4-yl]-5-propyl-3H-imidazo[2,1-b]purin-4-one (XXV), 5-propyl-2-[1-[3-[4-(trifluoromethyl)phenyl]prop-2-ynyl]pyrazol-4-yl]-3H-imidazo[2,1-b]purin-4-one (XXVI), 2-[1-[[1-(3-fluorophenyl)-5-oxo-pyrrolidin-3-yl]methyl]pyrazol-4-yl]-5-propyl-3H-imidazo[2,1-b]purin-4-one (XXVII), 2-[1-[[1-(m-tolyl)-5-oxo-pyrrolidin-3-yl]methyl]pyrazol-4-yl]-5-propyl-3H-imidazo[2,1-b]purin-4-one (XXVIII), 2-[1-[(3-chloro-5-fluoro-phenyl)methyl]pyrazol-4-yl]-5-propyl-3H-imidazo[2,1-b]purin-4-one (XXIX), 2-[[4-(4-oxo-5-propyl-3H-imidazo[2,1-b]purin-2-yl)pyrazol-1-yl] methyl] benzonitrile (XXX), 2-[1-[(2-fluorophenyl)methyl]pyrazol-4-yl]-5-propyl-3H-imidazo[2,1-b]purin-4-one (XXXI), 4-[[4-(4-oxo-5-propyl-3H-imidazo[2,1-b]purin-2-yl)pyrazol-1-yl]methyl]benzonitrile (XXXII), 2-[1-[[3-(4-methylpiperazin-1-yl)phenyl]methyl]pyrazol-4-yl]-5-propyl-3H-imidazo[2,1-b]purin-4-one (XXXIII), 2-[1-[1-(3-fluorophenyl)ethyl]pyrazol-4-yl]-5-propyl-3H-imidazo[2,1-b]purin-4-one (XXXIV), 2-[1-[(4-isopropylphenyl)methyl]pyrazol-4-yl]-5-propyl-3H-imidazo[2,1-b]purin-4-one (XXXV), 5-propyl-2-[1-(2,2,2-trifluoroethyl)pyrazol-4-yl]-3H-imidazo[2,1-b]purin-4-one (XXXVI), 2-[1-(2-aminoethyl)pyrazol-4-yl]-5-propyl-3H-imidazo[2,1-b]purin-4-one (XXXVII), 5-propyl-2-(1-tetrahydropyran-4-ylpyrazol-4-yl)-3H-imidazo[2,1-b]purin-4-one (XXXVIII), 2-(1-cyclopentylpyrazol-4-yl)-5-propyl-3H-imidazo[2,1-b]purin-4-one (XXXIX), 7-methyl-5-propyl-2-[1-[[3-(trifluoromethyl)phenyl]methyl]pyrazol-4-yl]-31H-imidazo[2,1-b]purin-4-one (XL), 8-methyl-5-propyl-2-[1-[[3-(trifluoromethyl)phenyl]methyl]pyrazol-4-yl]-3H-imidazo[2,1-b]purin-4-one (XLI), 7-methyl-5-propyl-2-(1-propylpyrazol-4-yl)-3H-imidazo[2,1-b]purin-4-one (XLII), 2-(1-ethylpyrazol-4-yl)-7-methyl-5-propyl-3H-imidazo[2,1-b]purin-4-one (XLIII), 7-methyl-2-(1-methylpyrazol-4-yl)-5-propyl-3H-imidazo[2,1-b]purin-4-one (XLIV), 5-propyl-2-(1-propylpyrazol-4-yl)-7-(trifluoromethyl)-3H-imidazo[2,1-b]purin-4-one (XLV), 5-propyl-7-(trifluoromethyl)-2-[1-[[3-(trifluoromethyl)phenyl]methyl]pyrazol-4-yl]-3H-imidazo[2,1-b]purin-4-one (XLVI), 2-[1-(m-tolylmethyl)pyrazol-4-yl]-4-propyl-1H-imidazo[2,1-f]purin-5-one (XLVII), 2-[1-[(3-fluorophenyl)methyl]pyrazol-4-yl]-4-propyl-1H-imidazo[2,1-f]purin-5-one (XLVIII), 3-[[4-(5-oxo-4-propyl-1H-imidazo[2,1-f]purin-2-yl)pyrazol-1-yl]methyl]benzonitrile (XLIX), 3-[[4-(4-ethyl-5-oxo-1H-imidazo[2,1-f]purin-2-yl)pyrazol-1-yl]methyl]benzonitrile (L), 3-[1-methyl-1-[4-(4-oxo-5-propyl-3H-imidazo[2,1-b]purin-2-yl)pyrazol-1-yl] ethyl] benzonitrile (LI), 5-propyl-2-[3-[[3-(trifluoromethyl)phenyl]methoxy]isoxazol-5-yl]-3H-imidazo[2,1-b]purin-4-one (LII).

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action.

Pharmaceutical compositions incorporating a compound described herein may take any physical form that is pharmaceutically acceptable. Pharmaceutical compositions for oral administration are particularly preferred. For example, such pharmaceutical compositions include, but are not limited to, tablets, troches, lozenges, aqueous or oily suspensions, dispersible powders or granules, emulsions, hard or soft capsules, or syrups or elixirs.

Known methods of formulating used in pharmaceutical science may be followed to prepare pharmaceutical compositions. All of the usual types of compositions are contemplated, including, but not limited to, tablets, chewable tablets, capsules, and solutions.

Capsules may be prepared by mixing a compound described herein with a suitable diluent and filling the proper amount of the mixture in capsules. Tablets may be prepared by direct compression, by wet granulation, or by dry granulation. Their formulations usually incorporate diluents, binders, lubricants, and disintegrators, as well as the compound described herein as an active therapeutic agent. A lubricant in a tablet formulation may help prevent the tablet and punches from sticking in the die. Tablet disintegrators are substances that swell when wetted to break up the tablet and release the compound. Enteric formulations are often used to protect an active ingredient from the strongly acidic contents of the stomach and to delay disintegration and absorption in the gastrointestinal tract. Such formulations are created by coating a solid dosage form with a film of a polymer that is insoluble in acid environments, and soluble in basic environments. Tablets are often coated with sugar as a flavor and sealant.

### Methods of Treatment, Dosing and Treatment Regimens

The compounds disclosed herein, or pharmaceutically acceptable salts, or solvates thereof, are useful for the treatment of cancer.

The term “cancer” as used herein, refers to an abnormal growth of cells that tend to proliferate in an uncontrolled way and, in some cases, to metastasize (spread). Types of cancer include, but are not limited to, solid tumors (such as those of the bladder, bowel, brain, breast, endometrium, heart, kidney, lung, liver, uterus, lymphatic tissue (lymphoma), ovary, pancreas or other endocrine organ (thyroid), prostate, skin (melanoma or basal cell cancer) or hematological tumors (such as the leukemias and lymphomas) at any stage of the disease with or without metastases.

In some embodiments, a mammal treated with a compound described herein has a disease or disorder that is or is associated with a cancer or tumor. Thus, in some embodiments, the mammal is a human that is an oncology patient. Such diseases and disorders and cancers include carcinomas, sarcomas, benign tumors, primary tumors, tumor metastases, solid tumors, non-solid tumors, blood tumors, leukemias and lymphomas, and primary and metastatic tumors.

In some embodiments, the A_{2B} adenosine receptor antagonists described herein (e.g., Compound 1 or prodrugs of Compound 1 (e.g., prodrugs of Formula (I), (II), (IIa), (III), (A), and/or (B)) are used as monotherapy in the treatment of cancer (e.g., carcinomas, sarcomas, benign tumors, primary tumors, tumor metastases, solid tumors, non-solid tumors, blood tumors, leukemias and lymphomas, hyperproliferative disorders, and/or primary and/or metastatic tumors as described herein).

Provided herein is a method for treating a cancer in a mammal, the method comprising administering to the mammal a prodrug of Compound 1, or a pharmaceutically acceptable salt or solvate thereof, wherein Compound 1 has the following structure:

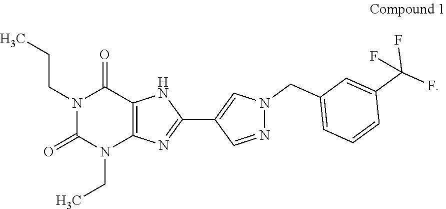 [SMILES: CCCn1c(=O)c2[nH]c(-c3cnn(Cc4cccc(C(F)(F)F)c4)c3)nc2n(CC)c1=O]

wherein the cancer is selected from bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, gastric cancer, rectal cancer, urothelial cancer, testis cancer, cervical cancer, head and neck cancer, and skin cancer. In some embodiments, the cancer is prostate cancer, breast cancer, colon cancer, or lung cancer. In some embodiments, the prostate cancer is castration resistant prostate cancer. In some embodiments, the breast cancer is triple negative breast cancer. In some embodiments, the prodrug of Compound 1, or a pharmaceutically acceptable salt or solvate thereof, has a structure described herein. In some embodiments, the cancer is a solid tumor. In some embodiments, the cancer is a lymphoma, a sarcoma, prostate cancer, or breast cancer.

In some embodiments, the A_{2B} adenosine receptor antagonists described herein are used alone or in combination with other agents in the treatment of solid tumors. A solid tumor is an abnormal mass of tissue that usually does not contain cysts or liquid areas. Solid tumors may be benign (not cancer), or malignant (cancer). Different types of solid tumors are named for the type of cells that form them. Examples of solid tumors are carcinomas, sarcomas, and lymphomas.

Carcinomas include, but are not limited to, esophageal carcinoma, hepatocellular carcinoma, basal cell carcinoma, squamous cell carcinoma, bladder carcinoma, bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, renal cell carcinoma, Wilm's tumor, cervical carcinoma, uterine carcinoma, testicular carcinoma, osteogenic carcinoma, epithelial carcinoma, and nasopharyngeal carcinoma.

Sarcomas include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Leukemias include, but are not limited to, a) chronic myeloproliferative syndromes (neoplastic disorders of multipotential hematopoietic stem cells); b) acute myelogenous leukemias; c) chronic lymphocytic leukemias (CLL), including B-cell CLL, T-cell CLL prolymphocyte leukemia, and hairy cell leukemia; and d) acute lymphoblastic leukemias (characterized by accumulation of lymphoblasts). Lymphomas include, but are not limited to, B-cell lymphomas (e.g., Burkitt's lymphoma); Hodgkin's lymphoma; and the like.

Benign tumors include, e.g., hemangiomas, hepatocellular adenoma, cavernous hemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

Primary and metastatic tumors include, e.g., lung cancer; breast cancer; colorectal cancer; anal cancer; pancreatic cancer; prostate cancer; ovarian carcinoma; liver and bile duct carcinoma; esophageal carcinoma; bladder carcinoma; carcinoma of the uterus; glioma, glioblastoma, medulloblastoma, and other tumors of the brain; kidney cancers; cancer of the head and neck; cancer of the stomach; multiple myeloma; testicular cancer; germ cell tumor; neuroendocrine tumor; cervical cancer; carcinoids of the gastrointestinal tract, breast, and other organs.

In one aspect, an A_{2B} adenosine receptor antagonist described herein, or a pharmaceutically acceptable salt or solvate thereof, reduces, ameliorates or inhibits immunosuppression and cell proliferation associated with cancers.

Provided herein are A_{2B} adenosine receptor antagonists that are useful for treating one or more diseases or disorders associated with or would benefit from modulation of A_{2B} adenosine receptor activity.

In some embodiments, described herein are methods for treating a disease or disorder, wherein the disease or disorder is cancer, or a hyperproliferative disorder.

In one embodiment, the compounds described herein, or a pharmaceutically acceptable salt thereof, are used in the preparation of medicaments for the treatment of diseases or conditions in a mammal that would benefit from inhibition or reduction of A_{2B} adenosine receptor activity. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a mammal already suffering from a disease or condition, in an amount sufficient to cure or at least partially arrest at least one of the symptoms of the disease or condition. Amounts effective for this use depend on the severity and course of the disease or condition, previous therapy, the mammal's health status, weight, and response to the drugs, and the judgment of a healthcare practitioner. Therapeutically effective amounts are optionally determined by methods including, but not limited to, a dose escalation and/or dose ranging clinical trial.

In prophylactic applications, compositions containing the compounds described herein are administered to a mammal susceptible to or otherwise at risk of a particular disease, disorder or condition. Such an amount is defined to be a “prophylactically effective amount or dose.” In this use, the precise amounts also depend on the mammal's state of health, weight, and the like. When used in mammals, effective amounts for this use will depend on the severity and course of the disease, disorder or condition, previous therapy, the mammal's health status and response to the drugs, and the judgment of a healthcare professional. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the mammal's condition does not improve, upon the discretion of a healthcare professional the administration of the compounds are administered chronically, that is, for an extended period of time, including throughout the duration of the mammal's life in order to ameliorate or otherwise control or limit the symptoms of the mammal's disease or condition.

In certain embodiments wherein a mammal's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the mammal requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one embodiment, doses employed for adult human treatment are from about 1 mg to about 1000 mg per day. In one embodiment, doses employed for adult human treatment are from about 1 mg to about 750 mg per day. In one embodiment, doses employed for adult human treatment are from about 1 mg to about 500 mg per day. In one embodiment, doses employed for adult human treatment are from about 1 mg to about 250 mg per day. In one embodiment, doses employed for adult human treatment are from about 1 mg to about 200 mg per day. In one embodiment, doses employed for adult human treatment are from about 1 mg to about 100 mg per day. In one embodiment, doses employed for adult human treatment are from about 1 mg to about 50 mg per day. In one embodiment, doses employed for adult human treatment are from about 1 mg to about 25 mg per day. In one embodiment, doses employed for adult human treatment are from about 1 mg to about 10 mg per day. In some of these embodiments, the dose relates to mg of Compound 1. In some of these embodiments, the dose relates to mg of a prodrug of Compound 1 described herein. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 100 mg/kg per body weight. In one embodiment, the daily dosages appropriate for the compound described herein, or a pharmaceutically acceptable salt thereof, are from about 0.01 to about 50 mg/kg per body weight. In one embodiment, the daily dosages appropriate for the compound described herein, or a pharmaceutically acceptable salt thereof, are from about 0.01 to about 25 mg/kg per body weight. In one embodiment, the daily dosages appropriate for the compound described herein, or a pharmaceutically acceptable salt thereof, are from about 0.01 to about 10 mg/kg per body weight. In one embodiment, the daily dosages appropriate for the compound described herein, or a pharmaceutically acceptable salt thereof, are from about 0.01 to about 5 mg/kg per body weight. In some of these embodiments, the dose relates to mg/kg per body weight of Compound 1. In some of these embodiments, the dose relates to mg/kg of Compound 1 as obtained after administration of a prodrug of Compound 1 described herein. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain instances, it is appropriate to administer at least one compound described herein, or a pharmaceutically acceptable salt thereof, in combination with one or more other therapeutic agents. In certain embodiments, the pharmaceutical composition further comprises one or more anti-cancer agents.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound described herein, or a pharmaceutically acceptable salt thereof, is co-administered with a second therapeutic agent, wherein the compound described herein, or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply be additive of the two therapeutic agents or the patient experiences a synergistic benefit.

In certain embodiments, different therapeutically-effective dosages of the compounds disclosed herein will be utilized in formulating pharmaceutical composition and/or in treatment regimens when the compounds disclosed herein are administered in combination with one or more additional agent, such as an additional therapeutically effective drug, an adjuvant or the like. Therapeutically-effective dosages of drugs and other agents for use in combination treatment regimens is optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. It also includes treatments in which a compound described herein, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the disease(s) for which relief is sought, is modified in accordance with a variety of factors (e.g. the disease or disorder from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

In some embodiments, a compound described herein, or a pharmaceutically acceptable salt thereof, is administered in combination with chemotherapy, radiation therapy, monoclonal antibodies, or combinations thereof.

Chemotherapy includes the use of anti-cancer agents.

### Immune Checkpoint Inhibitors

In some embodiments, a compound described herein (i.e. a A_{2B} adenosine receptor antagonist), or a pharmaceutically acceptable salt thereof, is administered in combination with an immune checkpoint inhibitor. In some embodiments, immune checkpoint inhibitors include, but are not limited to, anti-PD-1, anti-PD-L1, or anti-ligand 2 of programmed cell death protein 1 (PD-L2) agents/inhibitors. In some embodiments, immune checkpoint inhibitors include, but are not limited to anti-PD-1, anti-PD-L1, or anti-ligand 2 of programmed cell death protein 1 (PD-L2) antibodies.

As used herein, “PD-1” or “PD1” refers to the Programmed Death 1 (PD-1) receptor. Other names include programmed cell death protein 1 and CD279 (cluster of differentiation 279). PD-1 has two ligands, PD-L1 and PD-L2. In some embodiments, targeting PD-1 restores immune function in the tumor microenvironment.

As used herein, “PD-L1” or “PDL1” refers to the programmed death ligand 1 (PD-L1).

As used herein, “PD-L2” or “PDL2” refers to the programmed death ligand 2 (PD-L2).

In some embodiments, the anti-PD-1 or anti-PDL-1 agent is an antibody, a peptide, a small molecule or a nucleic acid.

In some embodiments, a compound described herein (i.e. a A_{2B} adenosine receptor antagonist), or a pharmaceutically acceptable salt thereof, is administered in combination with an anti-PD-1 or anti-PD-L1 agent. In some embodiments, the anti-PD-1 agent is an anti-PD-1 antibody. In some embodiments, the anti-PD-L1 agent is an anti-PD-L1 antibody.

In some embodiments, the anti PD-1 agent for use in combination with a compound described herein (i.e. a A_{2B} adenosine receptor antagonist), or a pharmaceutically acceptable salt thereof, is nivolumab, pembrolizumab, atezolizumab, durvalumab, pidilizumab, avelumab, TSR-042, PDR-001, tislelizumab (BGB-A317), cemiplimab (REGN2810), LY-3300054, JNJ-63723283, MGA012, BI-754091, IBI-308, camrelizumab (HR-301210), BCD-100, JS-001, CX-072, BGB-A333, AMP-514 (MEDI-0680), AGEN-2034, CSIOOI, Sym-021, SHR-1316, PF-06801591, LZM009, KN-035, AB122, genolimzumab (CBT-501), FAZ-053, CK-301, AK 104, or GLS-010, BGB-108, SHR-1210, PDR-001, PF-06801591, STI-1110, mDX-400, Spartalizumab (PDR001), Camrelizumab (SHR1210), Sintilimab (IBI308), Tislelizumab (BGB-A317), Toripalimab (JS 001), Dostarlimab (TSR-042, WBP-285), INCMGA00012 (MGA012), AMP-224, or AMP-514 (MEDI0680).

In some embodiments, the anti PD-1 agent is an anti PD-1 antibody.

“Anti-PD-1 antibody” refers to an antibody directed towards programmed death protein 1 (PD1). In some embodiments, an anti-PD-1 antibody binds an epitope of PD-1 which blocks the binding of PD-1 to any one or more of its putative ligands. In some embodiments, an anti-PD1 antibody binds an epitope of a PD-1 protein which blocks the binding of PD-1 to PD-L1 and/or PD-L2.

Exemplary anti-PD-1 antibodies include but are not limited to: nivolumab/MDX-1106/BMS-9300/ONO1152, a fully human lgG4 anti-PD-1 monoclonal antibody; pidilizumab (MDV9300/CT-011), a humanized lgG1 monoclonal antibody; pembrolizumab (MK-3475/pembrolizumab/lambrolizumab), a humanized monoclonal IgG4 antibody; durvalumab (MEDI-4736) and atezolizumab.

In some embodiments, the anti-PD-1 antibody is nivolumab (OPDIVO®, Bristol-Myers Squibb), pembrolizumab (KEYTRUDA®, Merck), cemiplimab (Libtayo), labrolizumab (Merck), or BGB-A317.

In some embodiments, the anti-PD1 antibody is an antibody set forth in U.S. Pat. Nos. 7,029,674, 7,488,802, 7,521,051, 8,008,449, 8,354,509, 8,617,546, 8,709,417, or WO2014/179664.-

The terms “antibody” and “antibodies” as used herein is inclusive of all types of immunoglobulins, including IgG, IgM, IgA, IgD, and IgE, or fragments thereof, that may be appropriate for the medical uses disclosed herein. The antibodies may be monoclonal or polyclonal and may be of any species of origin, including, for example, mouse, rat, rabbit, horse, or human. Antibody fragments that retain specific binding to the protein or epitope, for example, PD-L1 or PD-1, bound by the antibody used in the present disclosure are included within the scope of the term “antibody.” The antibodies may be chimeric or humanized, particularly when they are used for therapeutic purposes. Antibodies and antibody fragments may be obtained or prepared using various methods.

In some embodiments, the anti PD-1 agent for use in combination with a compound described herein (i.e. a A_{2B} adenosine receptor antagonist), or a pharmaceutically acceptable salt thereof, is atezolizumab, avelumab, AMP-224, MEDI-0680, RG-7446, GX-P2, durvalumab, KY-1003, KD-033, MSB-0010718C, TSR-042, ALN-PDL, STI-A1014, CX-072, BMS-936559, KN035, CK-301 (Checkpoint Therapeutics), AUNP12, CA-170 (Aurigene/Curis), MED14736, MSB0010718C, MDX 1105-01, and BMS-986189.

In some embodiments, the anti PD-L1 agent is an anti PD-L1 antibody.

“Anti-PD-L1 antibody” refers to an antibody directed towards programmed death ligand 1 (PD-L1).

Anti-PD-L1 antibodies for use in combination with a compound described herein (i.e. a A_{2B} adenosine receptor antagonist), or a pharmaceutically acceptable salt thereof, include: avelumab; BMS-936559, a fully human IgG4 antibody; atezolizumab (MPDL3280A/RG-7446), a human monoclonal antibody; MEDI4736; MSB0010718C, and MDX 1105-01.

In some embodiments, the anti-PD-L1 antibody is avelumab (Bavencio®, Merck KGA/Pfizer), durvalumab (AstraZeneca) and atezolizumab (TECENTRIQ®, Roche).

Additional exemplary antibodies include, but are not limited to, the antibodies set forth in U.S. Pat. Nos. 8,217,149, 8,383,796, 8,552,154 and 8,617,546.

Peptide anti-PD-1/PD-L1 agents include AUNP12 (a 29-mer peptide by Aurigene and Laboratoires Pierre Fabre), CA-170 (Aurigene/Curis), BMS-986189 (a macrocyclic peptide by BMS).

Small molecule anti-PD-1/PD-L1 agents include those described in WO/2020/086556, WO/2020/014643, WO/2019/204609, WO/2019/160882, WO/2018/195321, WO2018026971, US20180044329, US20180044305, US20180044304, US20180044303, US20180044350, US20180057455, US20180057486, US20180045142, WO20180044963, WO2018044783, WO2018009505, WO20180044329, WO2017066227, WO2017087777, US20170145025, WO2017079669, WO2017070089, US2017107216, WO2017222976, US20170262253, WO2017205464, US20170320875, WO2017192961, WO2017112730, US20170174679, WO2017106634, WO2017202744, WO2017202275, WO2017202273, WO2017202274, WO2017202276, WO2017180769, WO2017118762, WO2016041511, WO2016039749, WO2016142835, WO2016142852, WO2016142886, WO2016142894, and WO2016142833. In some embodiments, the small molecule anti-PD-1/PD-L1 agent is GS-4224. In some embodiments, GS-4224 is administered at about 400 mg to about 1000 mg. In some embodiments, immune checkpoint inhibitors include, and are not limited to, anti-T cell immunoglobulin and anti-immunoreceptor tyrosine-based inhibition motif (ITIM) domain (anti-TIGIT) agents such as BMS-986207, which is an anti-TIGIT monoclonal antibody. In some embodiments, immune checkpoint inhibitors include, and are not limited to, anti-lymphocyte activation gene-3 (anti-LAG3) agents such as relatlimab (BMS). In some embodiments, immune checkpoint inhibitors include, and are not limited to, anti-vascular endothelial growth factor (anti-VEGF) agents such as ranibizumab (Lucentis®) and bevacizumab (Avastin®).

### EXAMPLES

The following examples are provided for illustrative purposes only and not to limit the scope of the claims provided herein.

### Example 1—Pharmacokinetic Properties

Pharmacokinetic studies were carried out in Sprague Dawley rats. Exemplary compounds were administered orally by gavage to groups of three rats using a single oral dose of 5 mg/kg. Each oral dose was prepared as a suspension in 0.5% methylcellulose in water. Blood samples were obtained serially from each rat at 0, 15, 30 min, and then 1, 2, 4, 8, and 24 hrs post dose.

Concentrations of an administered compound and the corresponding metabolite (Compound 1) in rat plasma were determined by a HPLC tandem mass spectrometric (LC/MS/MS) method. 50 μL Plasma PPT by ISTD in MeOH/Acetonitrile (1:1, v/v). 200 μL of 5 ng/mL Terfenadine and Buspirone was added to in MeOH/Acetonitrile (1:1, v/v) and mixed well. 5 μL of MeOH was added to all samples and vortexed for 1 min and centrifuged at 4000 rpm for 15 mins. The supernatant was diluted 3× with water (with 0.1% FA) and injected for LC/MS/MS analysis.

| | Compound of Formula | | | |
|---|---|---|---|---|
| Compound | (A) or (B) | | Compound 1 | |
| Matrix | Plasma | | Plasma | |
| Standard Range | 1-1000 | ng/mL | 10-10000 | ng/mL |
| Regression | Linear | | Linear | |
| Weighting | 1/(x * x) | | 1/(x * x) | |
| LLOQ | 1 | ng/mL | 10 | ng/mL |

| | | |
|---|---|---|
| Internal | 5 ng/mL Terfenadine | 5 ng/mL Terfenadine |
| Standard | and Buspirone in MeOH/ | and Buspirone in MeOH/ |
| | Acetonitrile (1:1, v/v) | Acetonitrile (1:1, v/v) |

Quantification of compounds were achieved by mass spectrometry using Multiple Reaction Monitoring (MRM) mode, monitoring the transitions specific to each exemplary compound and 447.34>405.20 for Compound 1. The quantification limit of the assay was 10 ng/mL for Compound 1.

### Pharmacokinetic Analysis

Non-compartmental pharmacokinetic parameters were determined using a commercial program WinNonLin Professional, Version 8.0 (Pharsight, Mountain View, Calif). Plasma concentration at below level of detection was assumed to be Zero for the calculation of means and pharmacokinetic parameters.

For oral administration, t1/2 (hr), tmax (hr), Cmax (ng/mL), AUClast (hr*ng/mL), AUCInf (hr*ng/mL), AUC Extr (%), MRTInf (hr), Cmax Ratio (Parent/Pro), AUClast Ratio (Parent/Pro) were determined.

Table 3 describes exemplary AUCₗₐₛₜ data of Compound 1 for representative compounds of Formula (A).

TABLE 3

| Compound | AUC (hr*ng/mL) |
|---|---|
| I | α |
| J | α |
| F | β |
| M | γ |
| G | γ |
| H | β |
| O | β |
| P | β |
| S | β |

α = AUC is greater than or equal to 90,000 hr*ng/mL; β = AUC is less than 90,000 hr*ng/mL and greater than or equal to 30,000 hr*ng/mL; γ = AUC is less than 30,000 hr*ng/mL and greater than 10,000 hr*ng/mL.

Table 4 describes exemplary AUCₗₐₛₜ data of Compound 1 for representative compounds of Formula (B).

TABLE 4

| Compound | AUC (hr*ng/mL) |
|---|---|
| C | α |
| E | α or β |
| L | β |
| N | α |
| J | β |

α = AUC is greater than or equal to 90,000 hr*ng/mL; β = AUC is less than 90,000 hr*ng/mL and greater than or equal to 30,000 hr*ng/mL; γ = AUC is less than 30,000 hr*ng/mL and greater than 10,000 hr*ng/mL.

Table 5 describes exemplary AUCₗₐₛₜ data for Compound 1.

TABLE 5

| Compound | AUC (hr*ng/mL) |
|---|---|
| 1 | γ |

α = AUC is greater than or equal to 90,000 hr*ng/mL; β = AUC is less than 90,000 hr*ng/mL and greater than or equal to 30,000 hr*ng/mL; γ = AUC is less than 30,000 hr*ng/mL.

### Example 2—Combination with Anti-PD-1 in a CT26 Model

Methods: Balb/C inbred female mice, aged at 8-9 week, were purchased from Charles River. On the day of inoculation (Day 0), CT26 cells were harvested, washed and counted. Cells were re-suspended as single cell solution in PBS at a concentration of 5×10⁶ cells/mL at the final step. Immediately, five hundred thousand (5×10⁵) of CT26 cells suspended in 0.1 mL PBS were injected in the right flank of Balb/C mice subcutaneously using 27G needles. When palpable, tumors were measured by a caliper and tumor volume (mm³) were calculated by length×width×height×0.5236. Mice with the tumor size approximate to 100 mm³ were randomly assigned into one of four groups (n=10). Each group received vehicle (BID), A2B antagonist Compound 1 at 3 mg/kg (BID), mouse anti-PD-1 (RMP1-14) at 5 mg/kg (Q2D), or Compound 1 (3 mg/kg)+RMP1-14 (5 mg/kg) intraperitoneally for 16 days. Tumor size and body weight were determined every 2-3 days.

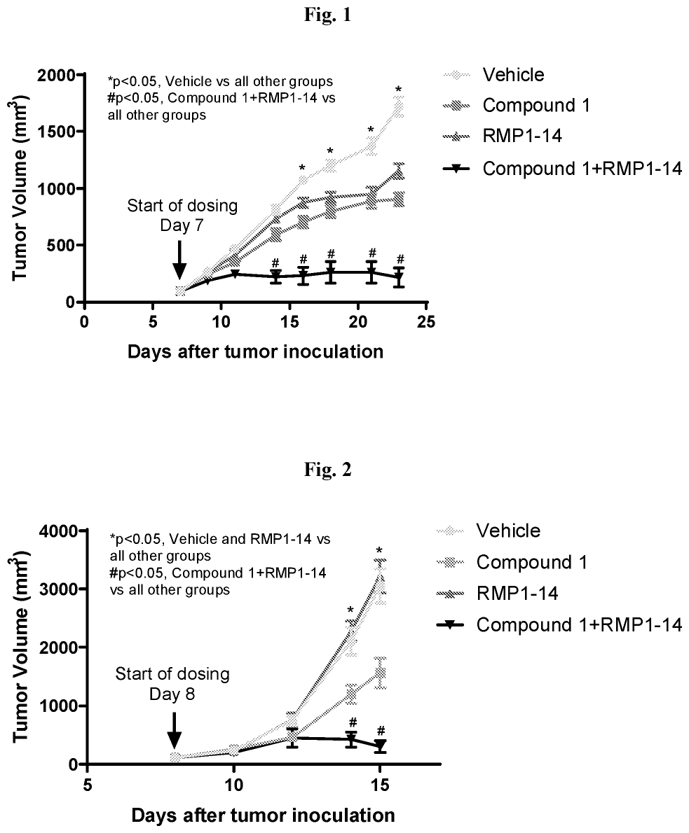

Results (see FIG. 1): Compound 1 and RMP1-14 treatment alone resulted in maximal CT26 tumor growth inhibition (TGI) by 47.5% and 33.3%, respectively. However, combination of Compound 1 with RMP1-14 produced synergistic effect on maximal TGI by 87.4%. Compound 1 and anti-PD-1 treatment had no significant effect on mouse body weight.

### Example 3—Combination with Anti-PD-1 in a B16F10 Model

Methods: C57BL/6 inbred female mice, aged at 8-9 week, were purchased from Charles River. On the day of inoculation (Day 0), B16F10 cells were harvested, washed and counted. Cells were re-suspended as single cell solution in PBS at a concentration of 5×10⁶ cells/mL at the final step. Immediately, five hundred thousand (5×10⁵) of B16F10 cells suspended in 0.1 mL PBS were injected in the right flank of C57BL/6 mice subcutaneously using 27G needles. When palpable, tumors were measured by a caliper and tumor volumes (mm³) were calculated by length×width×height×0.5236. Mice with the tumor size approximate to 100 mm³ were randomly assigned into one of four groups (n=10). Each group received vehicle (BID), A2B antagonist Compound 1 at 1 mg/kg (BID), mouse anti-PD-1 (RMP1-14) at 10 mg/kg (Q3D), or Compound 1 (1 mg/kg)+RMP1-14 (10 mg/kg) intraperitoneally for 7 days. Tumor size and body weight were determined every 2-3 days.

Results (see FIG. 2): Compound 1 treatment alone resulted in maximal B16F10 tumor growth inhibition (TGI) by 48.7%. By contrast, RMP1-14 had no effect (maximal TGI 4.6%). However, combination of Compound 1 with RMP1-14 produced synergistic effect on maximal TGI by 90.1%. Compound 1 and anti-PD-1 treatment had no effect on mouse body weight.

As demonstrated in Example 1, the prodrugs of Compound 1 can provide higher exposure of Compound 1 in animals. It is expected that these prodrugs can produce synergistic effect with anti-PD-1 therapy in tumor models as well.

### Example 4—Mechanistic Study of Combination with Anti-PD-1 in a MC38 Model

Methods: C57BL/6 inbred female mice, aged at 6-8 week, were purchased from Shanghai Lingchang Biotechnology Co., Ltd. On the day of inoculation (Day 0), MC38 cells were harvested, washed and counted. Cells were re-suspended as single cell solution in PBS at a concentration of 1×10⁶ cells/mL at the final step. Immediately, five hundred thousand (1×10⁵) of MC38 cells suspended in 0.1 mL PBS were injected in the right flank of C57BL/6 mice subcutaneously using 27G needles. When palpable, tumors were measured by a caliper and tumor volume (mm³) were calculated by length×width×height×0.5236. Mice with the tumor size approximately to 80-100 mm³ were randomly assigned into one of four groups (n=8). Each group received vehicle (BID), A2B antagonist Compound 1 at 3 mg/kg (BID), mouse anti-PD-1 (RMP1-14) at 10 mg/kg (Q3D), or Compound 1 (3 mg/kg)+RMP1-14 (10 mg/kg) intraperitoneally. Tumor tissues were collected 6 days after drug treatment and made single cell suspension for FACS analysis to detect tumor-infiltrating immune cells.

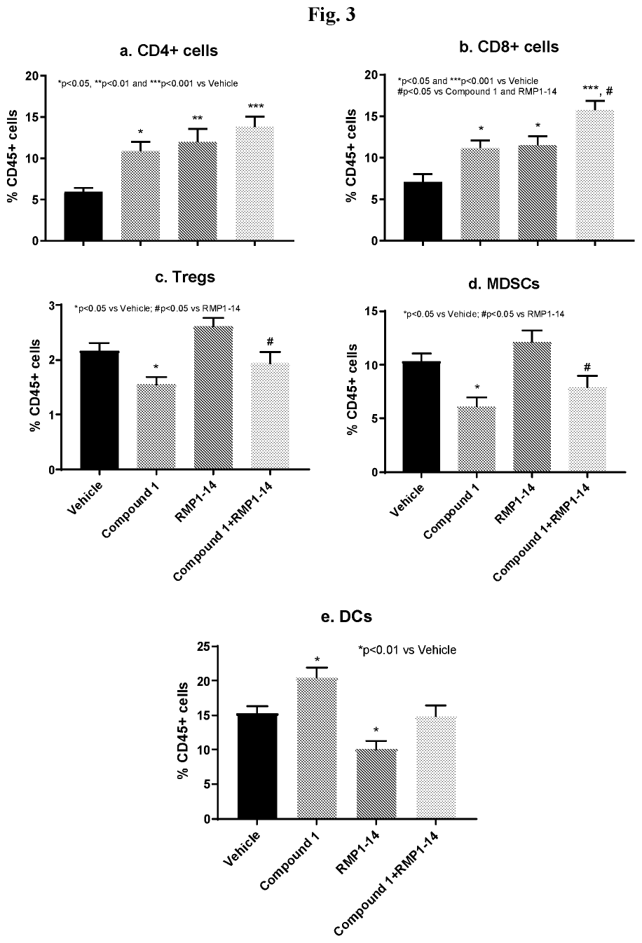

Results (see FIG. 3): Compound 1 significantly increased the amount of CD4⁺ and CD8⁺ T cells and dendritic cells (DCs) by 84%, 58% and 34%, respectively. In addition, it significantly reduced the amount of immunosuppressive cells regulatory T cells (Tregs) and myeloid-derived suppressor cells (MDSCs) by 28% and 41%, respectively. RMP1-14 significantly increased CD4⁺ and CD8⁺ T cells by 100% and 63%, respectively. By contrast, it reduced DCs by 34% and tended to increase immunosuppressive cells Tregs and MDSCs. However, combination of Compound 1 with RMP1-14 increased CD8⁺ T cells by 123% and normalized the negative effects of RMP1-14 on DCs, Tregs and MDSCs. This data suggest that Compound 1 can promote antitumor immunity and combination of Compound 1 with anti-PD-1 could result in synergistic effect on tumor growth inhibition in humans. Table 3 above describes exemplary AUCₗₐₛₜ data of Compound 1 for representative prodrugs of Compound 1, e.g., compounds of Formula (A).

It has been found that administration of a prodrug of Compound 1 enhances the overall bioavailability of Compound 1 with up to a five-fold increase for rat and dog.

### Example 5—Clinical Trial of Prodrug of Compound 1

This trial will be a Phase I/II trial of a compound of Formula (A) in patients with advanced solid tumors. The trial will evaluate a prodrug of Compound 1 as a monotherapy given in patients with advanced solid tumors. The trial will also evaluate a prodrug of Compound 1 in combination with programmed cell death protein 1 (PD-1)/programmed death-ligand 1 (PD-L1) directed immunotherapy in patients with advanced solid tumors.

A prodrug of Compound 1 will be administered orally with water only, once daily (QD) under modified fasting conditions on a continuous 21-day schedule (i.e. no breaks between cycles). Participants will be required to fast for two hours before dosing and up to at least one-hour post dose. The starting dose of a prodrug of Compound 1 will be 20 mg and patients may continue up to 18 cycles (i.e. one year).

Patient Population: Approximately 90 patients with DNA mismatch repair/Microsatellite instability/(MMR/MSI) defective tumors, triple negative breast cancer (TNBC) and metastatic castration resistant prostate cancer (mCRPC) will be enrolled in the monotherapy cohort with a starting dose of 20 mg per day. A dose escalation study will be conducted.

Approximately 90 patients will be enrolled in the combination therapy cohort. A PDL-1 antibody therapy will be administered in combination with a prodrug of Compound 1.

Endpoints: Identification of a dose that is deemed tolerable with a target dose limiting toxicity (DLT) rate of 20%, with a probability interval of (15%, 25%) using a Bayesian Continual Reassessment Method (CRM). Maximum tolerated dose/maximum administered dose (MTD/MAD) will be determined based on the incidence of defined DLTs.

Overall Response Rate (ORR) of confirmed Complete Response (CR) or Partial Response (PR). Prostate Cancer Working Group 3 (PCWG3) criteria (encompassing RECIST 1.1 and Prostate Specific Antigen (PSA) level changes) will be used in patients with metastatic castration resistant prostate cancer (mCRPC). Assess anti-tumor response according to RECIST Version 1.1. Assess progression free survival (PFS) at six months, and overall survival (OS) at 12 months.

While embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed. It is intended that the following claims define the scope of the disclosure and that methods and structures within the scope of these claims and their equivalents be covered thereby.

What is claimed is:

1. A method for treating cancer in a mammal, the method comprising administering to the mammal Compound 1, or a pharmaceutically acceptable salt or solvate thereof, or a prodrug of Compound 1, or a pharmaceutically acceptable salt or solvate thereof, and at least one immune checkpoint inhibitor, wherein Compound 1 has the following structure:

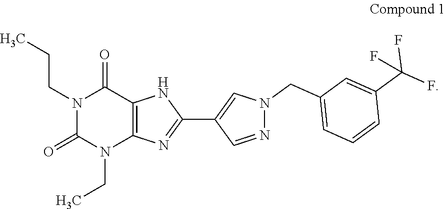 [SMILES: CCCn1c(=O)c2[nH]c(-c3cnn(Cc4cccc(C(F)(F)F)c4)c3)nc2n(CC)c1=O]

2. The method of claim 1, wherein the cancer is a solid tumor.

3. The method of claim 1, wherein the cancer is bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, gastric cancer, rectal cancer, urothelial cancer, testis cancer, cervical cancer, head and neck cancer, or skin cancer.

4. The method of claim 1, wherein the cancer is prostate cancer, breast cancer, colon cancer, or lung cancer.

5. The method of claim 1, wherein the cancer is castration resistant prostate cancer.

6. The method of claim 1, wherein the cancer is breast cancer.

7. The method of claim 1, wherein the cancer is a sarcoma, carcinoma, or lymphoma.

8. The method of any one of claims 1-7, wherein the immune checkpoint inhibitor is an anti-PD-1 agent or an anti-PD-L1 agent.

9. The method of claim 8, wherein the anti-PD-1 agent or anti-PD-L1 agent is nivolumab, pembrolizumab, cemiplimab, labrolizumab, avelumab, durvalumab or atezolizumab.

10. The method of any one of claims 1-9, wherein the mammal is a human.

11. The method of any one of claims 1-10, wherein the prodrug of Compound 1 has the following structure: 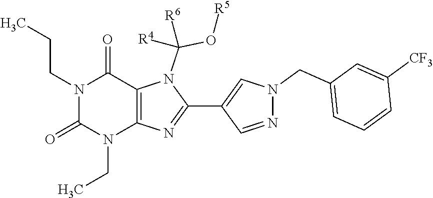
or a pharmaceutically acceptable salt or solvate thereof, wherein:
R⁴ is substituted or unsubstituted alkyl;
R⁶ is hydrogen or substituted or unsubstituted alkyl;
or R⁴ and R⁶ are taken together with the carbon atom to which they are attached to form a carbonyl (C═O);
or R⁴ and R⁶ are taken together with the carbon atom to which they are attached to form a ring that is a substituted or unsubstituted C₃-C₁₀cycloalkyl, or substituted or unsubstituted C₂-C₁₀heterocycloalkyl, wherein if the ring is substituted then it is substituted with one or more R⁵;
R¹⁵ is hydrogen, substituted or unsubstituted alkyl, substituted or unsubstituted heteroalkyl, substituted or unsubstituted phenyl, substituted or unsubstituted heteroaryl, -alkyl-(substituted or unsubstituted phenyl), -alkyl-(substituted or unsubstituted heteroaryl), —C(═O)R¹⁶, —C(═O)—OR¹⁶, —C(═O)N(R¹⁶)₂;
each R¹⁶ is independently selected from hydrogen and substituted or unsubstituted alkyl;
R⁵ is hydrogen, R⁷, —C(═O)R⁷, —C(═O)—OR⁷, —C(═O)N(R⁷)(R⁸), —C(═O)—SR⁷, or —P(═O)(OR⁹)₂;
or R⁴ and R⁵ are taken together with the atoms to which they are attached to form a substituted or unsubstituted C₂-C₁₀heterocycloalkyl;
R⁷ is substituted or unsubstituted alkyl, substituted or unsubstituted heteroalkyl, substituted or unsubstituted C₃-C₁₀cycloalkyl, substituted or unsubstituted C₂-C₁₀heterocycloalkyl, substituted or unsubstituted phenyl, substituted or unsubstituted heteroaryl, -alkyl-(substituted or unsubstituted phenyl), -alkyl-(substituted or unsubstituted heteroaryl), -alkyl-(substituted or unsubstituted cycloalkyl),-alkyl-(substituted or unsubstituted heterocycloalkyl), —(C(R¹⁰)₂O)ₘ—R¹¹, —(CH₂CH₂O)ₙ—R¹¹, or —(C(R¹⁰)₂)ₚ—OR¹¹;
R⁸ is hydrogen or alkyl;
or R⁷ and R⁸ are taken together with the nitrogen atom to which they are attached to form a substituted or unsubstituted C₂-C₁₀heterocycloalkyl;
each R⁹ is independently selected from hydrogen and alkyl;
each R¹⁰ is independently selected from hydrogen and alkyl;
R¹¹ is hydrogen, substituted or unsubstituted alkyl, substituted or unsubstituted heteroalkyl, substituted or unsubstituted C₂-C₁₀heterocycloalkyl, —C(═O)R¹², —C(═O)—OR¹², —C(═O)N(R¹²)(RI), —C(═O)—SR¹², or —P(═O)(OR⁹)₂;
R¹² is hydrogen, substituted or unsubstituted alkyl, substituted or unsubstituted heteroalkyl, substituted or unsubstituted C₃-C₁₀cycloalkyl, substituted or unsubstituted
C₂-C₁₀heterocycloalkyl, substituted or unsubstituted phenyl, substituted or unsubstituted heteroaryl, -alkyl-(substituted or unsubstituted phenyl), or -alkyl-(substituted or unsubstituted heteroaryl);
m is 1, 2, 3, 4, 5, or 6;
n is 1, 2, 3, 4, 5, or 6;
p is 1, 2, 3, 4, 5, or 6;
wherein substituted means that the referenced group is substituted with one or more additional groups individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone.

12. The method of claim 11, wherein:
R⁴ is methyl, ethyl, or n-propyl;
R⁶ is selected from hydrogen, methyl, ethyl, and n-propyl;
or R⁴ and R⁶ are taken together with the carbon atom to which they are attached to form a carbonyl (C═O).

13. The method of any one of claims 1-10, wherein the prodrug of Compound 1 has the following structure: 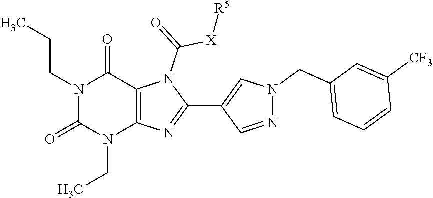
or a pharmaceutically acceptable salt or solvate thereof, wherein:
X is O, S, NHR′, NR′R″, CH₂, CHR′, or CR′R″;
R′ is C₁-C₆alkyl;
R″ is C₁-C₆alkyl;
R⁵ is hydrogen, R⁷, —C(═O)R⁷, —C(═O)—OR⁷, —C(═O)N(R⁷)(R⁸), —C(═O)—SR⁷, or —P(═O)(OR⁹)₂;
R⁷ is substituted or unsubstituted alkyl, substituted or unsubstituted heteroalkyl, substituted or unsubstituted C₃-C₁₀cycloalkyl, substituted or unsubstituted C₂-C₁₀heterocycloalkyl, substituted or unsubstituted phenyl, substituted or unsubstituted heteroaryl, -alkyl-(substituted or unsubstituted phenyl), -alkyl-(substituted or unsubstituted heteroaryl), -alkyl-(substituted or unsubstituted cycloalkyl),-alkyl-(substituted or unsubstituted heterocycloalkyl), —(C(R¹⁰)₂O)ₘ—R¹¹, —(CH₂CH₂O)ₙ—R¹¹, or —(C(R¹⁰)₂)ₚ—OR¹¹;
R⁸ is hydrogen or alkyl;
or R⁷ and R⁸ are taken together with the nitrogen atom to which they are attached to form a substituted or unsubstituted C₂-C₁₀heterocycloalkyl;
each R⁹ is independently selected from hydrogen and alkyl;
each R¹⁰ is independently selected from hydrogen and alkyl;
R¹¹ is hydrogen, substituted or unsubstituted alkyl, substituted or unsubstituted heteroalkyl, substituted or unsubstituted C₂-C₁₀heterocycloalkyl, —C(═O)R¹², —C(═O)—OR¹², —C(═O)N(R¹²)(R⁸), —C(═O)—SR¹², or —P(═O)(OR⁹)₂;
R¹² is hydrogen, substituted or unsubstituted alkyl, substituted or unsubstituted heteroalkyl, substituted or unsubstituted C₃-C₁₀cycloalkyl, substituted or unsubstituted
C₂-C₁₀heterocycloalkyl, substituted or unsubstituted phenyl, substituted or unsubstituted heteroaryl, -alkyl-(substituted or unsubstituted phenyl), or -alkyl-(substituted or unsubstituted heteroaryl);
m is 1, 2, 3, 4, 5, or 6;
n is 1, 2, 3, 4, 5, or 6;
p is 1, 2, 3, 4, 5, or 6;
wherein substituted means that the referenced group is substituted with one or more additional groups individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone.

14. The method of any one of claims 11-13,
R⁵ is R⁷;
R⁷ is C₁-C₆alkyl, substituted or unsubstituted C₁-C₆heteroalkyl, substituted or unsubstituted monocyclic C₃-C₈cycloalkyl, substituted or unsubstituted bicyclic C₅-C₁₀cycloalkyl, substituted or unsubstituted monocyclic C₂-C₈heterocycloalkyl, substituted or unsubstituted bicyclic C₅-C₁₀heterocycloalkyl, substituted or unsubstituted phenyl, substituted or unsubstituted monocyclic heteroaryl, —CH₂-(substituted or unsubstituted phenyl), —CH₂-(substituted or unsubstituted heteroaryl), —CH₂-(substituted or unsubstituted C₂-C₈heterocycloalkyl), —CH(R¹⁰)O—R¹¹, —(CH₂CH₂O)ₙ—R¹¹, or —(C(R¹⁰)₂)ₚ—OR¹¹;
each R¹⁰ is independently selected from hydrogen and methyl;
R¹¹ is hydrogen, C₁-C₆alkyl, substituted or unsubstituted C₁-C₆heteroalkyl, substituted or unsubstituted C₂-C₁₀heterocycloalkyl, —C(═O)R¹², —C(═O)—OR¹², —C(═O)N(R¹²)(R⁸), —C(═O)—SR¹², or —P(═O)(OR⁹)₂.

15. The method of claim 14, wherein:
R⁷ is C₁-C₆alkyl, substituted or unsubstituted C₁-C₆heteroalkyl, —CH₂-(substituted or unsubstituted phenyl), —CH₂-(substituted or unsubstituted heteroaryl), —CH₂-(substituted or unsubstituted C₂-C₈heterocycloalkyl), —CH(R¹⁰)O—R¹¹, or —(CH₂CH₂O)ₙ—R¹¹;
R¹⁰ is hydrogen and methyl;
R¹¹ is hydrogen, C₁-C₆alkyl, substituted or unsubstituted C₁-C₆heteroalkyl, substituted or unsubstituted C₂-C₁₀heterocycloalkyl, —C(═O)R¹², —C(═O)—OR¹², —C(═O)N(R¹²)(R⁸), —C(═O)—SR¹², or —P(═O)(OH)₂.

16. The method of any one of claims 1-11, wherein the prodrug of Compound 1 has one of the following structures: 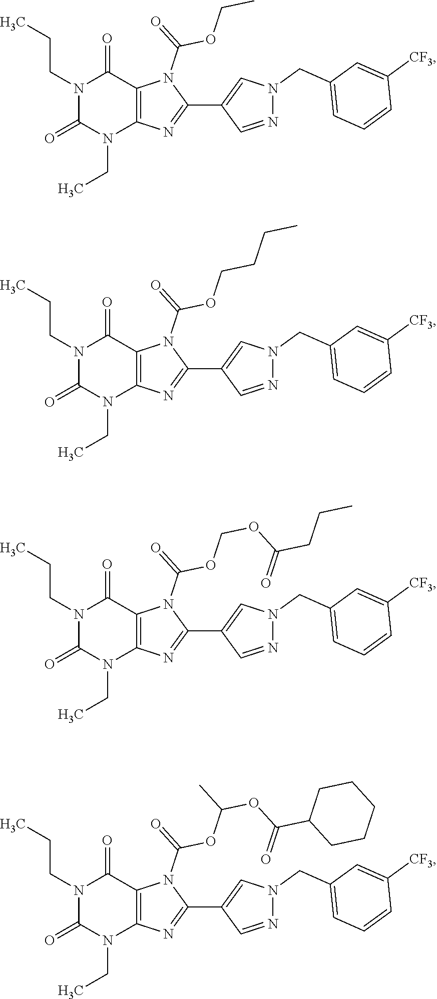 [SMILES: CCCn1c(=O)c2c(nc(-c3cnn(Cc4cccc(C(F)(F)F)c4)c3)n2C(=O)OCC)n(CC)c1=O ; CCCCOC(=O)n1c(-c2cnn(Cc3cccc(C(F)(F)F)c3)c2)nc2c1c(=O)n(CCC)c(=O)n2CC ; CCCC(=O)OCOC(=O)n1c(-c2cnn(Cc3cccc(C(F)(F)F)c3)c2)nc2c1c(=O)n(CCC)c(=O)n2CC ; CCCn1c(=O)c2c(nc(-c3cnn(Cc4cccc(C(F)(F)F)c4)c3)n2C(=O)OC(C)OC(=O)C2CCCCC2)n(CC)c1=O] 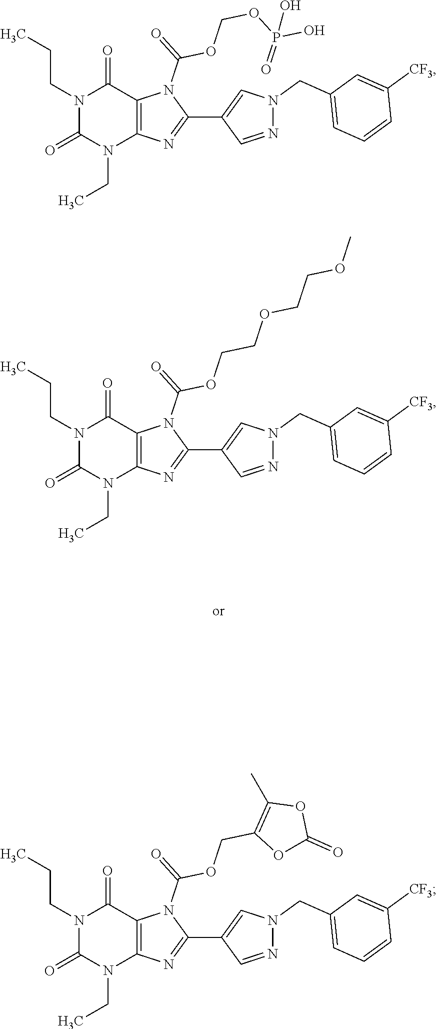 [SMILES: CCCn1c(=O)c2c(nc(-c3cnn(Cc4cccc(C(F)(F)F)c4)c3)n2C(=O)OCOP(=O)(O)O)n(CC)c1=O ; CCCn1c(=O)c2c(nc(-c3cnn(Cc4cccc(C(F)(F)F)c4)c3)n2C(=O)OCCOCCOC)n(CC)c1=O ; CCCn1c(=O)c2c(nc(-c3cnn(Cc4cccc(C(F)(F)F)c4)c3)n2C(=O)OCc2oc(=O)oc2C)n(CC)c1=O]
or a pharmaceutically acceptable salt or solvate thereof.

17. The method of claim 11, wherein the prodrug of Compound 1 has the following structure: 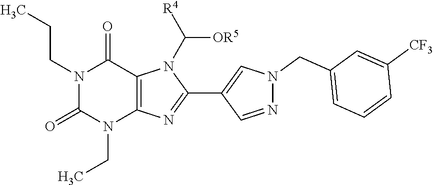
or a pharmaceutically acceptable salt or solvate thereof.

18. The method of claim 17, wherein:
R⁴ is methyl or ethyl;
R⁵ is R⁷, —C(═O)R⁷, —C(═O)—OR⁷, —C(═O)N(R⁷)(R⁸), —C(═O)—SR⁷, or —P(═O)(OH)₂;
R⁷ is C₁-C₆alkyl, substituted or unsubstituted C₁-C₆heteroalkyl, substituted or unsubstituted cyclohexyl, substituted or unsubstituted cyclopentyl, substituted or unsubstituted bicyclo[1.1.1]pentanyl, substituted or unsubstituted bicyclo[2.2.1]heptanyl, substituted or unsubstituted bicyclo[2.2.2]octanyl, substituted or unsubstituted bicyclo[3.2.1]octanyl, substituted or unsubstituted bicyclo[3.3.0]octanyl, substituted or unsubstituted bicyclo[4.3.0]nonanyl, or substituted or unsubstituted decalinyl, substituted or unsubstituted oxetanyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, substituted or unsubstituted thiomorpholinyl, substituted or unsubstituted phenyl, substituted or unsubstituted monocyclic heteroaryl, —CH₂-(substituted or unsubstituted phenyl), —CH₂-(substituted or unsubstituted heteroaryl), —CH₂-(substituted or unsubstituted C₂-C₈heterocycloalkyl), —CH(R¹⁰)O—R¹¹, —(CH₂CH₂O)ₙ—R¹¹, or —(C(R¹⁰)₂)ₚ—OR¹¹;
each R¹⁰ is independently selected from hydrogen and methyl;
R¹¹ is hydrogen, C₁-C₆alkyl, substituted or unsubstituted C₁-C₆heteroalkyl, substituted or unsubstituted C₂-C₁₀heterocycloalkyl, —C(═O)R¹², —C(═O)—OR¹², —C(═O)N(R¹²)(R⁸), —C(═O)—SR¹², or —P(═O)(OR⁹)₂.

19. The method of any one of claims 1-11, wherein the prodrug of Compound 1 has one of the following structures: 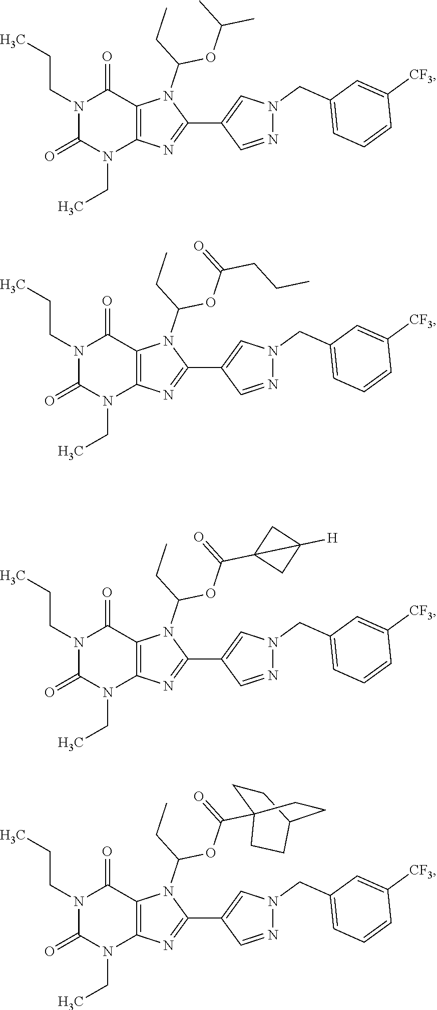 [SMILES: CCCn1c(=O)c2c(nc(-c3cnn(Cc4cccc(C(F)(F)F)c4)c3)n2C(CC)OC(C)C)n(CC)c1=O ; CCCC(=O)OC(CC)n1c(-c2cnn(Cc3cccc(C(F)(F)F)c3)c2)nc2c1c(=O)n(CCC)c(=O)n2CC ; CCCn1c(=O)c2c(nc(-c3cnn(Cc4cccc(C(F)(F)F)c4)c3)n2C(CC)OC(=O)C23CC2C3)n(CC)c1=O ; CCCn1c(=O)c2c(nc(-c3cnn(Cc4cccc(C(F)(F)F)c4)c3)n2C(CC)OC(=O)C23CCC(CC2)CC3)n(CC)c1=O] 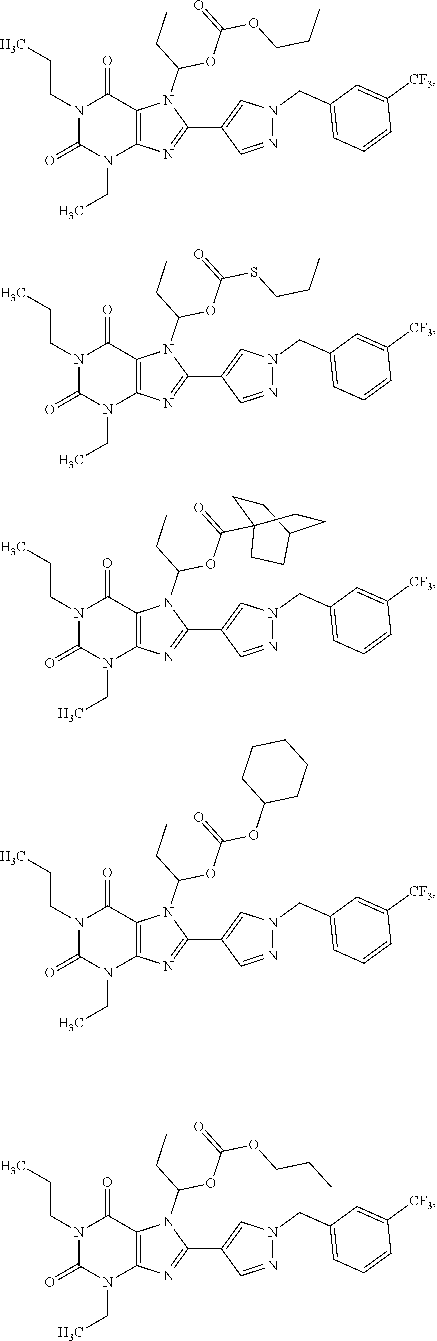 [SMILES: CCCOC(=O)OC(CC)n1c(-c2cnn(Cc3cccc(C(F)(F)F)c3)c2)nc2c1c(=O)n(CCC)c(=O)n2CC ; CCCSC(=O)OC(CC)n1c(-c2cnn(Cc3cccc(C(F)(F)F)c3)c2)nc2c1c(=O)n(CCC)c(=O)n2CC ; CCCn1c(=O)c2c(nc(-c3cnn(Cc4cccc(C(F)(F)F)c4)c3)n2C(CC)OC(=O)C23CCC(CC2)CC3)n(CC)c1=O ; CCCn1c(=O)c2c(nc(-c3cnn(Cc4cccc(C(F)(F)F)c4)c3)n2C(CC)OC(=O)OC2CCCCC2)n(CC)c1=O ; CCCOC(=O)OC(CC)n1c(-c2cnn(Cc3cccc(C(F)(F)F)c3)c2)nc2c1c(=O)n(CCC)c(=O)n2CC] 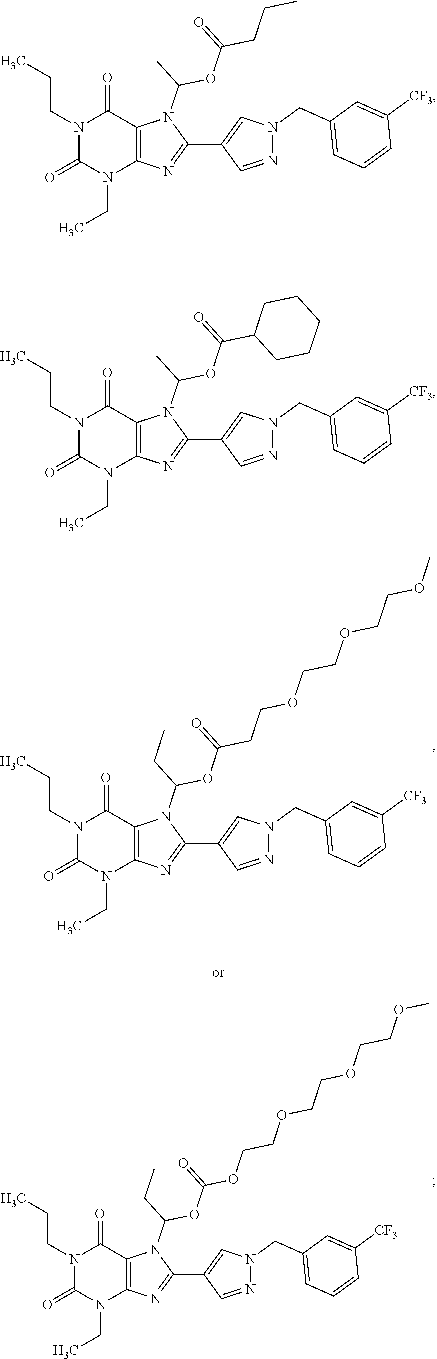
or a pharmaceutically acceptable salt or solvate thereof.

20. A method for treating a cancer in a mammal, the method comprising administering to the mammal a prodrug of Compound 1, or a pharmaceutically acceptable salt or solvate thereof, wherein Compound 1 has the following structure: 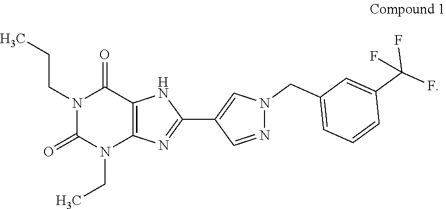 [SMILES: CCCn1c(=O)c2[nH]c(-c3cnn(Cc4cccc(C(F)(F)F)c4)c3)nc2n(CC)c1=O]
wherein the cancer is selected from bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, gastric cancer, rectal cancer, urothelial cancer, testes cancer, cervical cancer, vaginal cancer, vulvar cancer, head and neck cancer, and skin cancer.

21. The method of claim 20, wherein the cancer is prostate cancer, breast cancer, colon cancer, or lung cancer.

22. The method of claim 21, wherein the prostate cancer is castration resistant prostate cancer.

23. The method of claim 21, wherein the breast cancer is triple negative breast cancer.

24. The method of any one of claims 20-23, wherein the prodrug of Compound 1, or a pharmaceutically acceptable salt or solvate thereof, has a structure according to any one of claims 11-19.
